SEQUENCE LISTING (54 sequences)

SEQ ID NO: 1 - protein, 114 aa, Artificial Sequence
  XBR2-401 VH sequence
QSVKESEGGLFKPTDTLTLTCTVSGFSLSSYGVTWVRQAPGSGLEWIGYINTAGNTYYASWAKSRSTITRNTNENTVTLKMTSLTAADTATYFCARDWTSLNIWGPGTLVTVSS

SEQ ID NO: 2 - protein, 110 aa, Artificial Sequence
  XBR2-401 VL sequence
DPMLTQTPSSTSTAVGDTVTIKCQASQSISSDLSWYQQKPGQRPKLLIYQASTLASGVPSRFKGSGYGTEYTLTISGVQREDAAIYYCLGGYADASYRTAFGGGTKLEIK

SEQ ID NO: 3 - protein, 115 aa, Artificial Sequence
  X3.12 VH sequence
QSVKESEGGLFKPTDTLTLTCTVSGFSLSSYGVTWVRQAPGSGLEWIGYINTAGNTYYASWAKSRSTITRNTNENTVTLKMTSLTAADTATYFCARDDRWSLNIWGPGTLVTVSS

SEQ ID NO: 4 - protein, 110 aa, Artificial Sequence; identical to SEQ ID NO: 2
  X3.12 VL sequence

SEQ ID NO: 5 - protein, 116 aa, Artificial Sequence
  hX3.12.5 VH sequence
EVQLVESGGGLIQPGGSLRLSCAASGFTVSSYGVTWVRQAPGKGLEWVSYINTAGNTYYASWAKSRFTISRDNSKNTLYLQMNSLRAEDTAVYYCARDDRWSLNIWGQGTLVTVSS

SEQ ID NO: 6 - protein, 110 aa, Artificial Sequence
  hX3.12.5 VL sequence
DPMLTQSPSSLSASVGDRVTITCQASQSISSDLSWYQQKPGKAPKLLIYQASTLASGVPSRFKGSGYGTEYTLTISSLQPEDFATYYCLGGYADASYRTAFGGGTKLEIK

SEQ ID NO: 7 - protein, 116 aa, Artificial Sequence
  hX3.12.6 VH sequence
EVQLVESGGGLVQPGGSLRLSCAASGFTFSSYGVTWVRQAPGKGLEWVSYINTAGNTYYASWAKSRFTISRDNAKNSLYLQMNSLRAEDTAVYYCARDDRWSLNIWGQGTLVTVSS

SEQ ID NO: 8 - protein, 15 aa, Artificial Sequence
  4(GS)x3 linker
GGGGSGGGGSGGGGS

SEQ ID NO: 9 - protein, 232 aa, Artificial Sequence
  hinge, CH2 (aglycosylation mutation) and CH3 (N297A)
EPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGA

SEQ ID NO: 10 - protein, 120 aa, Artificial Sequence
  V9 scFV heavy chain variable domain (VH)
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTV

SEQ ID NO: 11 - protein, 17 aa, Artificial Sequence
  (G4S)x3 linker
SSGGGGSGGGGSGGGGS

SEQ ID NO: 12 - protein, 107 aa, Artificial Sequence [sequence omitted]

SEQ ID NO: 13 - protein, 232 aa, Artificial Sequence
  hinge, CH2 (aglycosylation mutation) and CH3 (N297A)
EPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGA

SEQ ID NO: 14 - protein, 22 aa, Artificial Sequence
  GMCSF signal peptide
MLLLVTSLLLCELPHPAFLLIP

SEQ ID NO: 15 - protein, 4 aa, Artificial Sequence
  Partial amino acid sequence of human ROR1 from Figure 8
FKSD

SEQ ID NO: 16 - protein, 12 aa, Artificial Sequence
  IgG4 hinge
ESKYGPPCPPCP

SEQ ID NO: 17 - protein, 28 aa, Artificial Sequence
  CD28 transmembrane domain
MFWVLVVVGGVLACYSLLVTVAFIIFWV

SEQ ID NO: 18 - protein, 112 aa, Artificial Sequence
  CD3z signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 24 aa, Artificial Sequence
  T2A ribosomal skipping sequence
LEGGGEGRGSLLTCGDVEENPGPR

SEQ ID NO: 20 - protein, 335 aa, Artificial Sequence
  EGFRt
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 21 - protein, 19 aa, Artificial Sequence
  Human Ig heavy chain signal peptide
MDWTWRILFLVAAATGAHS

SEQ ID NO: 22 - protein, 79 aa, Artificial Sequence
  Partial amino acid sequence of human ROR2
CYNGSGMDYRGTASTTKSGHQCQPWALQHPHSHHLSSTDFPELGGGHAYCRNPGGQMEGPWCFTQNKNVRMELCDVPSC

SEQ ID NO: 23 - protein, 7 aa, Artificial Sequence
  HCDR3 sequence from Figure 9
  VARIANT (2)..(3): X is an amino acid which can be selected from any naturally occurring amino acid
DXXSLNI

SEQ ID NO: 24 - protein, 8 aa, Artificial Sequence
  HCDR3 sequence from Figure 9
  VARIANT (2)..(4): X is an amino acid which can be selected from any naturally occurring amino acid
DXXXSLNI

SEQ ID NO: 25 - protein, 9 aa, Artificial Sequence
  HCDR3 sequence from Figure 9
  VARIANT (2)..(5): X is an amino acid which can be selected from any naturally occurring amino acid
DXXXXSLNI

SEQ ID NO: 26 - protein, 8 aa, Artificial Sequence
  HCDR3 sequence from Figure 10
DDRWSLNI

SEQ ID NO: 27 - protein, 8 aa, Artificial Sequence
  HCDR3 sequence from Figure 10
DKGWSLNI

SEQ ID NO: 28 - protein, 7 aa, Artificial Sequence
  HCDR3 sequence from Figure 10
DTMSLNI

SEQ ID NO: 29 - protein, 9 aa, Artificial Sequence
  HCDR3 sequence from Figure 10
DWGNWSLNI

SEQ ID NO: 30 - protein, 7 aa, Artificial Sequence
  HCDR3 sequence from Figure 10
DYTSLNI

SEQ ID NO: 31 - protein, 7 aa, Artificial Sequence
  HCDR3 sequence from Figure 10
DSMSLNI

SEQ ID NO: 32 - protein, 8 aa, Artificial Sequence
  HCDR3 sequence from Figure 10
DGLTSLNI

SEQ ID NO: 33 - protein, 9 aa, Artificial Sequence
  HCDR3 sequence from Figure 10
DYMMNSLNI

SEQ ID NO: 34 - protein, 8 aa, Artificial Sequence
  HCDR3 sequence from Figure 10
DSRNSLNI

SEQ ID NO: 35 - protein, 9 aa, Artificial Sequence
  HCDR3 sequence from Figure 10
DSGVVSLNI

SEQ ID NO: 36 - protein, 7 aa, Artificial Sequence
  HCDR3 sequence from Figure 10
DWTSLNI

SEQ ID NO: 37 - protein, 9 aa, Artificial Sequence
  HCDR3 sequence from Figure 10
DNGSTSLNI

SEQ ID NO: 38 - protein, 9 aa, Artificial Sequence
  HCDR3 sequence from Figure 10
DSRRKSLNI

SEQ ID NO: 39 - protein, 110 aa, Artificial Sequence
  VL1 sequence from Figure 11
DIQMTQSPSSLSASVGDRVTITCQASQSISSDLSWYQQKPGKAPKLLLYQASTLASGVPSRFSGSGSGTDYTLTISSLQPEDFATYYCLGGYADASYRTAFGGGTKLEIK

SEQ ID NO: 40 - protein, 116 aa, Artificial Sequence; identical to SEQ ID NO: 5
  VH1 sequence from Figure 11

SEQ ID NO: 41 - protein, 11 aa, Artificial Sequence
  LCDR1 sequence from Figure 11
QASQSISSDLS

SEQ ID NO: 42 - protein, 7 aa, Artificial Sequence
  LCDR2 sequence from Figure 11
QASTLAS

SEQ ID NO: 43 - protein, 12 aa, Artificial Sequence
  LCDR3 sequence from Figure 11
LGGYADASYRTA

SEQ ID NO: 44 - protein, 5 aa, Artificial Sequence
  HCDR1 sequence from Figure 11
SYGVT

SEQ ID NO: 45 - protein, 16 aa, Artificial Sequence
  HCDR2 sequence from Figure 11
YINTAGNTYYASWAKS

SEQ ID NO: 46 - DNA, 46 bp, Artificial Sequence
  STOP primer
acctatttctgtgcgaggattgtaatcccttaacatctggggacca

SEQ ID NO: 47 - DNA, 21 bp, Artificial Sequence
  Unirev primer
atctctcgcacagaaataggt

SEQ ID NO: 48 - DNA, 48 bp, Artificial Sequence
  X2 primer
  misc_difference (22)..(23): n is a or g or c or t
  misc_feature (22)..(23): n is a, c, g, or t
  misc_feature (25)..(26): n is a, c, g, or t
acctatttctgtgcgagagatnnknnktcccttaacatctggggacca

SEQ ID NO: 49 - DNA, 51 bp, Artificial Sequence
  X3 primer
  misc_difference (22)..(23): n is a or g or c or t
  misc_feature (22)..(23): n is a, c, g, or t
  misc_feature (25)..(26): n is a, c, g, or t
  misc_feature (28)..(29): n is a, c, g, or t
acctatttctgtgcgagagatnnknnknnktcccttaacatctggggacca

SEQ ID NO: 50 - DNA, 54 bp, Artificial Sequence
  X4 primer
  misc_difference (22)..(23): n is a or g or c or t
  misc_feature (22)..(23): n is a, c, g, or t
  misc_feature (25)..(26): n is a, c, g, or t
  misc_feature (28)..(29): n is a, c, g, or t
  misc_feature (31)..(32): n is a, c, g, or t
acctatttctgtgcgagagatnnknnknnknnktcccttaacatctggggacca

SEQ ID NO: 51 - protein, 6 aa, Artificial Sequence
  Further partial amino acid sequence of human ROR1 from Figure 8
TFTALR

SEQ ID NO: 52 - protein, 8 aa, Artificial Sequence
  HCDR3 sequence from Figure 11
DDRWSLNI

SEQ ID NO: 53 - protein, 4 aa, Artificial Sequence
  Partial amino acid sequence of VhX3.12 and Vh401 from Figure 11
QSVK

SEQ ID NO: 54 - protein, 4 aa, Artificial Sequence
  Partial amino acid sequence of Vh3, VhX3.12 and Vh401 from Figure 11
NTNE

(19) United States
(12) Patent Application Publication
(10) Pub. No.: US 2023/0143003 A1
(43) Pub. Date: May 11, 2023

(54) AFFINITY MATURED AND HUMANIZED BINDING DOMAINS TARGETING ROR2

(71) Applicant: JULIUS-MAXIMILIANS-UNIVERSITÄT WÜRZBURG, Würzburg (DE)

(72) Inventors: Michael HUDECEK, Höchberg (DE); Justus WEBER, Würzburg (DE); Rebecca S. GOYDEL, Palm Beach Gardens, FL (US); Christoph RADER, Jupiter, FL (US)

(21) Appl. No.: 17/910,327

(22) PCT Filed: Mar. 12, 2021
(86) PCT No.: PCT/EP2021/056349

§ 371 Date: Sep. 8, 2022

(30) Foreign Application Priority Data
Mar. 13, 2020 (EP) 20163019.1

(51) Int. Cl. C07K 16/28; A61P 35/00; C07K 14/705

(52) U.S. Cl. CPC C07K 16/2803; C07K 16/2809; A61P 35/00; C07K 14/70521; C07K 14/70578; C07K 2317/565; C07K 2317/92; C07K 2317/31; C07K 2317/24; C07K 2317/55; C07K 2317/40; C07K 2317/622; C07K 2319/33; A61K 38/00

(57) ABSTRACT

The invention relates to antibodies and derivatives thereof such as bispecific antibodies and chimeric antigen receptors (CARs) with affinity matured and/or humanized targeting domains specific to the ROR2 antigen. The invention encompasses the nucleic acids and vectors encoding said antibodies and derivatives, the cells and pharmaceutical compositions containing them, in particular for their use in cancer therapy.

### FIELD OF THE INVENTION

The invention relates to antibodies and derivatives thereof such as bispecific antibodies and chimeric antigen receptors (CARs) with affinity matured and/or humanized targeting domains specific to the ROR2 antigen. The invention encompasses the nucleic acids and vectors encoding said antibodies and derivatives, the cells and pharmaceutical compositions containing them, in particular for their use in cancer therapy.

### BACKGROUND OF THE INVENTION

CAR-T Cells

The adoptive transfer of genetically modified T cells that express a T cell receptor or a chimeric antigen receptor (CAR) specific for a tumor-associated antigen is emerging as an effective modality for cancer therapy [1-5]. CARs are synthetic receptors most often constructed by linking a single chain variable fragment (scFV) of a monoclonal antibody (mAb) specific for a tumor cell surface molecule to a transmembrane domain, one or more intracellular costimulatory signaling modules, and CD3ζ [6-8]. CAR-modified T-cells confer non-MHC restricted recognition of tumor cells, and durable responses have been reported in patients with B-cell malignancies after treatment with autologous T-cells modified with CARs specific for the B-cell lineage restricted CD19 molecule. The major toxicities in these patients were related to tumor lysis, cytokine release, and prolonged depletion of normal B-lymphocytes [1-3, 5, 9].

ROR2 as a Novel Target for Cancer Therapy:

Monoclonal antibodies (mAbs) are widely used for cancer immunotherapy with approximately 30 antibody-based cancer therapies FDA-approved and marketed [10]. Infrequent identification of new suitable cancer antigens restricts the indications and patients suited for mAb therapies. Due to their overexpression on cancer cells, receptor tyrosine kinases (RTKs) have proven their general suitability as cancer antigens.

One RTK not yet targeted by FDA-approved mAbs is ROR2 (receptor tyrosine kinase-like orphan receptor 2), which is expressed during embryogenesis and tightly downregulated in postnatal tissues [11-13]. A number of solid and hematologic malignancies have been shown to express ROR2, suggesting its suitability as a target for antibody-based cancer therapies [14].

Recently, the inventors have proposed ROR2 as a candidate for cancer immunotherapy. ROR2 is expressed during embryonic development and plays an important role for neural and skeletal development. It has been shown to be involved in the WNT signaling pathway when associated with its ligand WNT5A and facilitates polarization of cells during embryonic development along with regulating migration and differentiation [15-18].

While largely downregulated after birth in mice [19] and humans [20, 21], ROR2 is overexpressed in several cancers [14, 15] including solid malignancies such as renal cell adenocarcinoma and subsets of breast cancer, and hematologic malignancies, such as multiple myeloma [22-25]. Among solid malignancies without FDA-approved and marketed antibody-based cancer therapies, a notable indication is sarcoma where ROR2 overexpression was found in osteosarcoma, leiomyosarcoma, and gastrointestinal stromal tumor (GIST) [21, 26]. Numerous studies show ROR2 expression correlates with rapid disease progression, tumor invasiveness and metastases, making ROR2 a promising cancer target and biomarker. [24, 27, 28]

Recently, ROR2-targeting campaigns were translated from preclinical to clinical investigations (NCT03504488, NCT03393936, NCT03960060), this underscores the suitability and attraction of ROR2 as a candidate antigen for antibody-based cancer therapy.

However, there remains a need in the art for more effective cancer therapies and for more effective therapeutic agents which can be used in such therapies.

### DESCRIPTION OF THE INVENTION

The present invention inter alia relates to affinity matured and humanized binding domains of the known anti-ROR2 antibody XBR2-401 and the uses thereof for the construction of bi-specific antibodies (bi-mAbs), and antibody derivatives such as CARs and CAR engineered T cells. See, for instance, reference [29, 30] for a description of the XBR2-401 antibody, which is hereby incorporated by reference in its entirety for all purposes. The bi-mAbs and CARs in the present invention have a higher binding affinity and/or degree of “human-ness” as compared to the parental rabbit XBR2-401 binding domains. According to the invention, these bi-mAbs, CARs and CAR engineered T cells are expected to exhibit higher efficacy and/or lower immunogenicity in clinical use in patients, as opposed to the rabbit (XBR2-401) binding domain.

The new affinity matured and humanized monoclonal antibodies (mAbs) that target the kringle domain of human ROR2 (ROR2-Kr) were developed from the prior art rabbit mAb XBR2-401 [29, 30] based on an unpublished co-crystal structure of XBR2-401 in scFv format in complex with ROR2-Kr. The co-crystal structure provided a molecular picture of the interaction and defined both the paratope (the antigen binding site of the antibody) and the epitope (the antibody binding site of the antigen) at high resolution (1.2 Å). Surprisingly, the molecular picture of the paratope/epitope interface revealed an open cavity in the interaction of the third complementarity-determining region of the heavy chain (HCDR3) with the epitope. The inventors reasoned that this gap could be filled by adding and replacing amino acid residues in a short stretch of the HCDR3 sequence. Doing so, they proposed, would increase the affinity and maintain the specificity of XBR2-401. Thus, the short stretch in HCDR3 of XBR2-401 was subjected to amino acid residue randomization that included length variation, i.e. inclusion of zero, one, and two additional randomized positions. The library was subsequently selected by phage display technology for binding to human ROR2. This combined rational design and in vitro evolution strategy yielded a panel of new mAbs with HCDR3 sequences deviating from mAb XBR2-401 in terms of both amino acid sequence composition and length. Subsequent analyses by surface plasmon resonance and cell microarray technology, respectively, confirmed the intended increase in affinity without loss of specificity. One of the new mAbs, named XBR2-401-X3.12, was humanized by rational design to yield XBR2-401-hX3.12.5 and XBR2-401-hX3.12.6. The latter was again co-crystallized with ROR2-Kr and revealed the intended tighter interaction of paratope and epitope by closing the open cavity. Collectively, the surprising findings of the present inventors, which led to the new mAbs claimed in the current invention, were based on acquiring and analyzing extensive new data (X-ray crystallography structures of XBR2-401 in complex with ROR2-Kr) that were not available as prior art. The rationale for the teachings of the invention, e.g., filling a particular open cavity in the paratope-epitope interaction, was confirmed by X-ray crystallography structures of XBR2-401-hX3.12.6 in complex with ROR2-Kr.

According to the invention, affinity maturation can be carried out by any method that is known in the art. As a non-limiting example, affinity maturation of the VH and VL domains of XBR2-401 has been performed by random mutagenesis of defined complementarity determining region areas.

According to the invention, humanization can be carried out by any method that is known in the art. As a non-limiting example, humanization of the VH and VL domains of X3.12 monoclonal antibody has been performed by CDR grafting and screening of best binders for the ROR2 target by ELISA, flow cytometry, and surface plasmon resonance.

According to the invention, recombinant mammalian cells expressing CARs such as CAR T cells can be produced to express CARs with the affinity matured and/or humanized anti-ROR2 binding domains of monoclonal anti-ROR2 antibodies X3.12, hX3.12.5 and hX3.12.6.

Affinity maturation and humanization of ROR2-specific CARs is different from the known clinical approaches which rely on non-humanized ROR2-specific CARs. The present inventors have surprisingly shown that affinity matured ROR2-specific CARs have higher functionality than their non-affinity matured counterparts.

Furthermore, the inventors have surprisingly shown that humanized, affinity matured ROR2 bi-mAbs that show advantageous functional properties can be produced according to the invention.

Accordingly, the present invention provides the following preferred embodiments:
1. An antibody capable of binding to human ROR2 or a derivative thereof capable of binding to human ROR2, wherein the antibody or derivative thereof comprises a light chain variable domain and a heavy chain variable domain, wherein the heavy chain variable domain comprises a CDR3 sequence having an amino acid sequence selected from the group consisting of SEQ ID NO: 23, SEQ ID NO: 24 and SEQ ID NO: 25, and wherein said CDR3 sequence does not have the amino acid sequence of SEQ ID NO: 36.
2. The antibody or derivative thereof of item 1, wherein the heavy chain variable domain comprises a CDR3 sequence having an amino acid sequence selected from the group consisting of SEQ ID NO: 24 and SEQ ID NO: 25.
3. The antibody or derivative thereof of any one of the preceding items, wherein the heavy chain variable domain comprises a CDR3 sequence having the amino acid sequence of SEQ ID NO: 24.
4. The antibody or derivative thereof of item 1 or 2, wherein the heavy chain variable domain comprises a CDR3 sequence having an amino acid sequence selected from the group consisting of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 37, and SEQ ID NO: 38.
5. The antibody or derivative thereof of item 1 or 2, wherein the heavy chain variable domain comprises a CDR3 sequence having an amino acid sequence selected from the group consisting of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35.
6. The antibody or derivative thereof of item 4, wherein the heavy chain variable domain comprises a CDR3 sequence having the amino acid sequence of SEQ ID NO: 26.
7. The antibody or derivative thereof of any one of the preceding items, wherein the light chain variable domain comprises a CDR3 sequence having the amino acid sequence of SEQ ID NO: 43 or a CDR3 sequence which differs from the amino acid sequence of SEQ ID NO: 43 by not more than two amino acid residues.
8. The antibody or derivative thereof of item 7, wherein the light chain variable domain comprises a CDR3 sequence having the amino acid sequence of SEQ ID NO: 43 or a CDR3 sequence which differs from the amino acid sequence of SEQ ID NO: 43 by not more than one amino acid residue.
9. The antibody or derivative thereof of item 7, wherein the light chain variable domain comprises a CDR3 sequence having the amino acid sequence of SEQ ID NO: 43.
10. The antibody or derivative thereof of any one of the preceding items, wherein the heavy chain variable domain comprises a CDR2 sequence having the amino acid sequence of SEQ ID NO: 45 or a CDR2 sequence which differs from the amino acid sequence of SEQ ID NO: 45 by not more than two amino acid residues.
11. The antibody or derivative thereof of item 10, wherein the heavy chain variable domain comprises a CDR2 sequence having the amino acid sequence of SEQ ID NO: 45 or a CDR2 sequence which differs from the amino acid sequence of SEQ ID NO: 45 by not more than one amino acid residue.
12. The antibody or derivative thereof of item 10, wherein the heavy chain variable domain comprises a CDR2 sequence having the amino acid sequence of SEQ ID NO: 45.
13. The antibody or derivative thereof of any one of the preceding items, wherein the light chain variable domain comprises a CDR1 sequence having the amino acid sequence of SEQ ID NO: 41 or a CDR1 sequence which differs from the amino acid sequence of SEQ ID NO: 41 by not more than two amino acid residues.
14. The antibody or derivative thereof of item 13, wherein the light chain variable domain comprises a CDR1 sequence having the amino acid sequence of SEQ ID NO: 41 or a CDR1 sequence which differs from the amino acid sequence of SEQ ID NO: 41 by not more than one amino acid residue.
15. The antibody or derivative thereof of item 13, wherein the light chain variable domain comprises a CDR1 sequence having the amino acid sequence of SEQ ID NO: 41.
16. The antibody or derivative thereof of any one of the preceding items, wherein the light chain variable domain comprises a CDR2 sequence having the amino acid sequence of SEQ ID NO: 42 or a CDR2 sequence which differs from the amino acid sequence of SEQ ID NO: 42 by not more than two amino acid residues.
17. The antibody or derivative thereof of item 16, wherein the light chain variable domain comprises a CDR2 sequence having the amino acid sequence of SEQ ID NO: 42 or a CDR2 sequence which differs from the amino acid sequence of SEQ ID NO: 42 by not more than one amino acid residue.
18. The antibody or derivative thereof of item 16, wherein the light chain variable domain comprises a CDR2 sequence having the amino acid sequence of SEQ ID NO: 42.
19. The antibody or derivative thereof of any one of the preceding items, wherein the heavy chain variable domain comprises a CDR1 sequence having the amino acid sequence of SEQ ID NO: 44 or a CDR1 sequence which differs from the amino acid sequence of SEQ ID NO: 44 by not more than two amino acid residues.
20. The antibody or derivative thereof of item 19, wherein the heavy chain variable domain comprises a CDR1 sequence having the amino acid sequence of SEQ ID NO: 44 or a CDR1 sequence which differs from the amino acid sequence of SEQ ID NO: 44 by not more than one amino acid residue.
21. The antibody or derivative thereof of item 19, wherein the heavy chain variable domain comprises a CDR1 sequence having the amino acid sequence of SEQ ID NO: 44.
22. The antibody or derivative thereof of any one of items 1-3 and 5-21, wherein said antibody or derivative is capable of binding to human ROR2 with higher affinity than a corresponding antibody or derivative comprising a light chain variable domain having the amino acid sequence of SEQ ID NO: 2 and a heavy chain variable domain having the amino acid sequence of SEQ ID NO: 1, as determined by surface plasmon resonance measurements.
23. The antibody or derivative thereof of any one of the preceding items, wherein (i) the heavy chain variable domain comprises the amino acid sequence of SEQ ID NO: 3 and the light chain variable domain comprises the amino acid sequence of SEQ ID NO: 4, or (ii) the heavy chain variable domain comprises the amino acid sequence of SEQ ID NO: 5 and the light chain variable domain comprises the amino acid sequence of SEQ ID NO: 6, or (iii) the heavy chain variable domain comprises the amino acid sequence of SEQ ID NO: 7 and the light chain variable domain comprises the amino acid sequence of SEQ ID NO: 6.
24. The antibody or derivative thereof of any one of the preceding items, which is a humanized antibody or derivative thereof.
25. The antibody or derivative thereof of any one of the preceding items, wherein (ii) the heavy chain variable domain comprises the amino acid sequence of SEQ ID NO: 5 and the light chain variable domain comprises the amino acid sequence of SEQ ID NO: 6, or (iii) the heavy chain variable domain comprises the amino acid sequence of SEQ ID NO: 7 and the light chain variable domain comprises the amino acid sequence of SEQ ID NO: 6.
26. The antibody or derivative thereof of any one of the preceding items, which is a bispecific antibody or derivative thereof.
27. The antibody or derivative thereof of item 26, wherein the bispecific antibody or derivative thereof is also capable of binding to human CD3.
28. The antibody or derivative thereof of any one of the preceding items, which is a derivative of the antibody.
29. The derivative of item 28, wherein the derivative is an antibody fragment.
30. The derivative of item 29, wherein said antibody fragment is a Fab or a bispecific scFv-Fc.
31. The derivative of item 28, wherein the derivative is a CAR.
32. The derivative of item 28, wherein the derivative comprises an adaptor for a universal CAR.
33. A nucleic acid encoding the antibody or derivative of any one of items 1-32.
34. The nucleic acid of item 33, wherein the nucleic acid is an mRNA.
35. The nucleic acid of item 33, wherein the nucleic acid is a DNA.
36. The nucleic acid of item 35, wherein the DNA is a minicircle or plasmid DNA.
37. A recombinant immune cell comprising the CAR of item 31 and/or comprising a nucleic acid according to any one of items 33-36 which encodes the CAR of item 31.
38. The recombinant immune cell of item 37, wherein said immune cell is a CD8+ killer T cell, a CD4+ helper T cell, a naive T cell, a memory T cell, a central memory T cell, an effector memory T cell, a memory stem T cell, an invariant T cell, an NKT cell, a cytokine induced killer T cell, a g/d T cell, a natural killer cell, a monocyte, a macrophage, a dendritic cell, or a granulocyte.
39. The recombinant immune cell of item 37 or 38, wherein the immune cell is a T-cell.
40. A pharmaceutical composition comprising:

(i) the antibody or derivative thereof of any one of items 1-30 or 32;

(ii) a nucleic acid according to any one of items 33-36 which encodes the CAR of item 31;

(iii) the recombinant immune cell of any one of items 37-39; or

(iv) a combination of (i) and (ii), a combination of (i) and (iii), or a combination of (i) to (iii).
41. A pharmaceutical composition according to item 40, for use in the treatment of cancer.
42. The pharmaceutical composition for use of item 41, wherein the cancer is a ROR2-expressing cancer.
43. The pharmaceutical composition for use of item 41 or 42, wherein the cancer is a hematologic cancer.
44. The pharmaceutical composition for use of item 41 or 42, wherein the cancer is a solid cancer.
45. The pharmaceutical composition for use of any one of items 41-44, wherein the cancer is selected from the group consisting of multiple myeloma, renal cell carcinoma, pancreatic cancer, sarcoma, glioblastoma and mammary carcinoma.
46. Use of a compound for the diagnosis of cancer, wherein the compound is
   (i) the antibody or derivative thereof of any one of items 1-30 or 32;
   (ii) a nucleic acid according to any one of items 33-36 which encodes the CAR of item 31;
   (iii) the recombinant immune cell of any one of items 37-39; or
   (iv) a combination of (i) and (ii), a combination of (i) and (iii), or a combination of (i) to (iii).
47. Use of a compound for determining the susceptibility of a cancer to therapy, wherein the compound is
   (i) the antibody or derivative thereof of any one of items 1-30 or 32;
   (ii) a nucleic acid according to any one of items 33-36 which encodes the CAR of item 31;
   (iii) the recombinant immune cell of any one of items 37-39; or
   (iv) a combination of (i) and (ii), a combination of (i) and (iii), or a combination of (i) to (iii).

### BRIEF DESCRIPTION OF THE DRAWINGS

FIG. 1: Amino acid sequences of the parental XBR2-401 VH and VL sequences and the complete 401×V9-bi-mAb coding sequence.

(A) Parental XBR2-401 VH and VL amino acid sequences

(B) Complete amino acid sequence of the 401×V9 bi-mAb, represented in an N- to C-terminal order.

(C) Complete amino acid sequence of the 401 CAR, represented in an N- to C-terminal order. Note that the asterisk denotes the end of the amino acid sequence.

FIG. 2: Amino acid sequences of the affinity matured X3.12 VH and VL sequences and the complete X3.12 CAR coding sequence.

(A) Affinity matured X3.12 VH and VL amino acid sequences

(B) Complete amino acid sequence of the X3.12×V9 bi-mAb, represented in an N- to C-terminal order. Note that the asterisk denotes the end of the amino acid sequence.

(C) Complete amino acid sequence of the X3.12 CAR, represented in an N- to C-terminal order. Note that the asterisk denotes the end of the amino acid sequence.

FIG. 3: Amino acid sequences of the humanized hX3.12.5 VH and VL sequences, the complete hX3.12.5 bi-mAb sequence, as well as the complete hX3.12.5 CAR coding sequence.

(A) Affinity matured and humanized hX3.12.5 VH and VL amino acid sequences

(B) Complete amino acid sequence of the hX3.12.5×V9 bi-mAb, represented in an N- to C-terminal order.

(C) Complete amino acid sequence of the hX3.12.5 CAR, represented in an N- to C-terminal order. Note that the asterisk denotes the end of the amino acid sequence.

FIG. 4: Amino acid sequences of the humanized hX3.12.6 VH and VL sequences, the complete hX3.12.6 bi-mAb sequence, as well as the complete hX3.12.6 CAR coding sequence.

(A) Affinity matured and humanized hX3.12.6 VH and VL amino acid sequences

(B) Complete amino acid sequence of the hX3.12.6×V9 bi-mAb, represented in an N- to C-terminal order.

(C) Complete amino acid sequence of the hX3.12.6 CAR, represented in an N- to C-terminal order. Note that the asterisk denotes the end of the amino acid sequence.

FIG. 5: Crystal structures of parental mAb 401 and affinity matured and humanized mAb hX3.12.6 in complex with hROR2-Kr. (A) (Top) The crystal structure of scFv 401 (VH and VL ribbons) in complex with hROR2-Kr (space-filling) was determined by X-ray crystallography at 1.2-Å resolution (PDB ID 60SH). The complex on the right is rotated 90° to make interactions of LCDR3 and HCDR3 with the epitope visible. (Bottom) Zoomed-in image of the 401:hROR2-Kr complex reveals HCDR3 residue Trp96 weakly hydrogen-bonding (dotted line) with the backbone of hROR2-Kr residue His348 with a distance of 3.4 Å. (B) (Top) Crystal structure of affinity matured and humanized scFv hX3.12.6 (VH (black) and VL (light gray)) in complex with hROR2-Kr at 1.4-Å resolution (PDB ID 60SV). (Bottom) Zoomed-in image of the hX3.12.6:hROR2-Kr complex reveals increased hydrogen (left dotted line) (2.8 Å) and π-π/π-cation (right dotted line) (4.2 Å) bonding of HCDR3 residue Trp98 with the backbone of hROR2-Kr residue His348 and His349, respectively. The heavy chain variable domain (V_{H}) is shown in darker shade while the light chain variable domain (V_{L}) is shown in lighter shade.

FIG. 6: Table of crystal structure parameters for complexes 401:hROR2-Kr and hX3.12.6:hROR2-Kr and unbound hROR2-Kr.

FIG. 7: Table of residue interactions between hROR2-Kr and 401 or hX3.12.6 determined using crystal structures.

FIG. 8: Comparison of 401:hROR2-Kr, hX3.12.6:hROR2-Kr, and R11:hROR1-Kr crystal structures, epitopes, and kringle domains. (A) Visual depiction where epitopes can be compared between the anti-ROR2 and anti-ROR1 mAbs. Darker and lighter shades denote VH and V_{L} domains, respectively. The ROR2 and ROR1 kringle domains are shown in darker and lighter shades of orange, respectively. The overall rmsd of Ca positions between the 401:hROR2-Kr and hX3.12.6:hROR2-Kr complexes was found to be 0.474 Å. (B) Comparison of co-crystallized kringle domains of ROR2 and ROR1 from left to right: Crystal structure of hROR2-Kr based on the crystallized 401:hROR2-Kr complex. The epitope of 401 is marked darker grey. Crystal structure of hROR1-Kr based on the crystallized R11:hROR1-Kr complex. The epitope of R11 is marked in light grey. Overlay of unbound hROR2-Kr (light gray) and hROR2-Kr from the 401:hROR2-Kr complex (dark gray). The rmsd of Ca positions was 0.383 Å. An acetate ion is bound to Arg385 of unbound hROR2-Kr through mixed salt bridge/hydrogen bond interactions. (C) Alignment of mouse, human, and monkey ROR2 and ROR1 kringle domain amino acid sequences (numbering from uniprot.org for hROR2). Residues that comprise the epitopes of 401 and R11 are marked. hROR2-Kr epitope residues which are not listed in Table I, which is contained in FIG. 7, interact with 401 and hX3.12.6 through van der Waals interactions.

FIG. 9: Table depicting construction of focused randomized HCDR3 phage libraries.

FIG. 10: Table of kinetic data of top 12 HCDR3 affinity matured anti-ROR2 clones.

FIG. 11: Amino acid sequence alignment of V_{L} and VH of the affinity matured and humanized variants. The location of the 4 framework regions (FWRs) and the 3 CDRs is indicated. The numbers refer to Kabat numbering of variable domain residues shown in single letter code. Residues underlined were back mutated to the original rabbit residue. Dots represent identical residues in the alignment to V_{L}1 or V_{H}1. CDR residues are shown in bold. (Top) The V_{L}1 FWRs are derived from human germline IGKV1-NL1*01; the V_{L}1 CDRs are grafted from X3.12. The V_{L}2 amino acid sequence is the same as V_{L}1 but with 7 back mutations from the human germline to the original rabbit residues of X3.12. (Bottom) The V_{H}1 and V_{H}2 FWRs are derived from human germlines IGHV3-66*03 and IGHV3-48*03 respectively and their CDRs are grafted from X3.12. V_{H}3 and V_{H}4 vary from V_{H}1 and V_{H}2 by back-mutating FWR residues from the human germline to the original rabbit residues of X3.12.

FIG. 12: Table of kinetic data of humanized anti-ROR2 mAbs.

FIG. 13: Analysis of affinity matured and humanized mAbs by flow cytometry.

(A) HEK 293F cells stably transfected with human ROR2 (allotype Thr245) were stained with the indicated parental, affinity matured, and humanized Fabs followed by PE-conjugated goat anti-human F(ab′)2 pAbs. Mock transfected HEK 293F cells served as negative control. The Fabs were also tested against T47D (ROR2+, ROR1−), 786-O (ROR2+, ROR1+) and MDA-MB-231 (ROR2−, ROR1+) cell lines. Humanized anti-human CD3 Fab v9 and secondary antibody alone (“background”) served as additional negative controls.

(B) After its conversion from Fab to scFv-Fc, hX3.12.6 followed by Alexa Fluor 647-conjugated donkey anti-human F(ab′)2 pAbs was used to stain 786-O, T47D, and MDA-MB-231 cell lines. Secondary antibody alone served as negative control

(C) Flow cytometry using hX3.12.6 scFv-Fc followed by Alexa Fluor 647-conjugated donkey anti-human F(ab′)2 pAbs for staining HEK 293F cells stably transfected with human ROR2 (allotype Thr245) or mouse ROR2. Mock transfected HEK 293F cells and secondary antibody alone (“background”) served as negative controls. All events were normalized to mode.

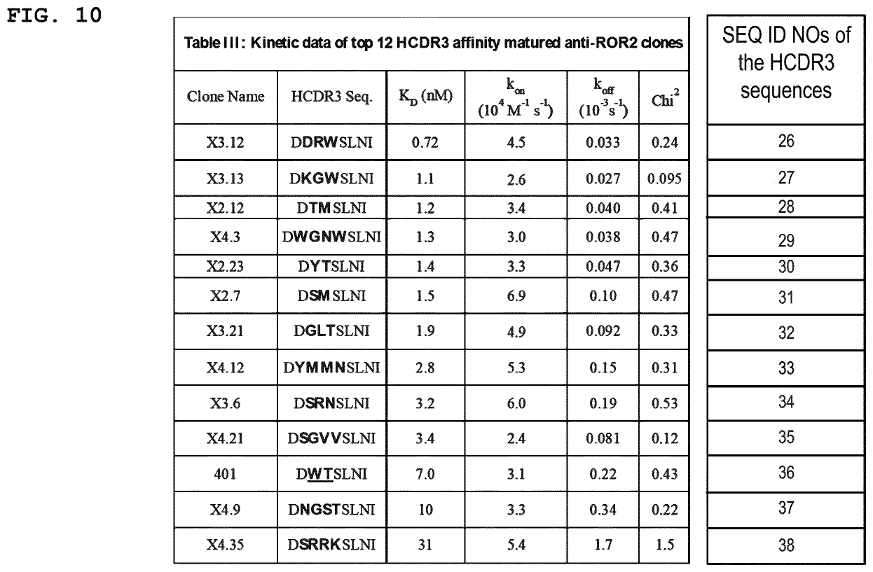

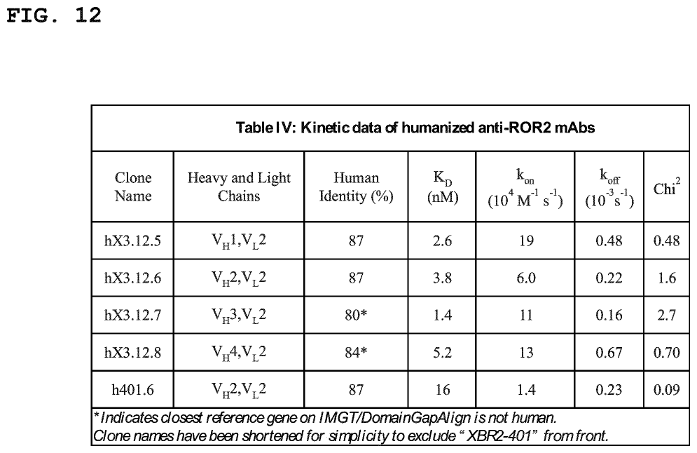

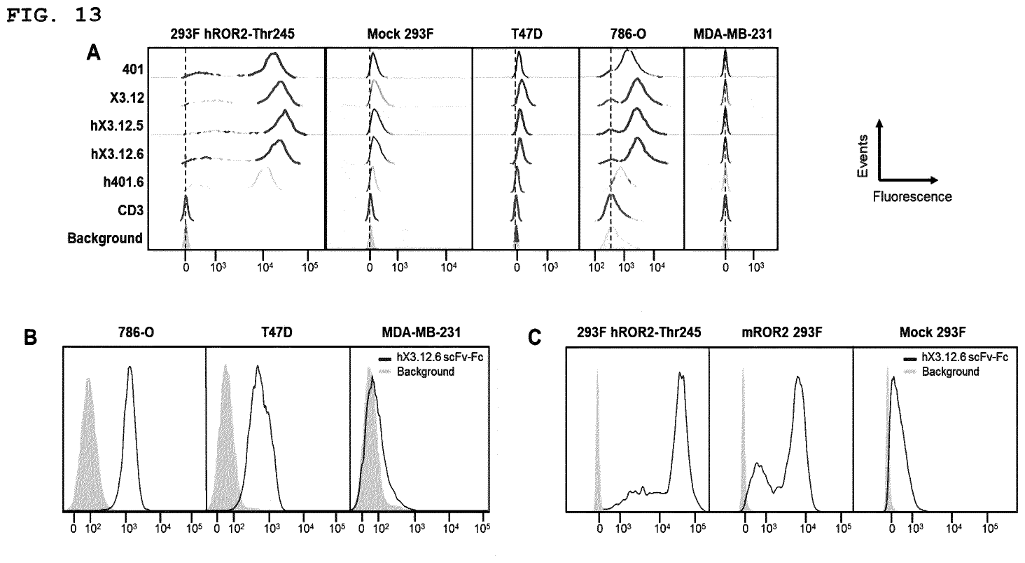

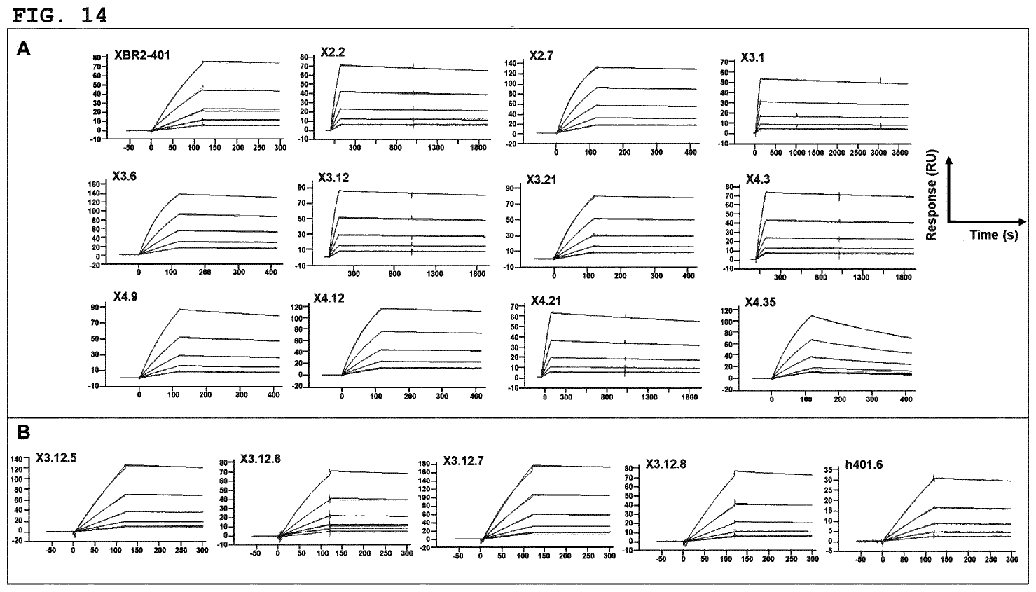

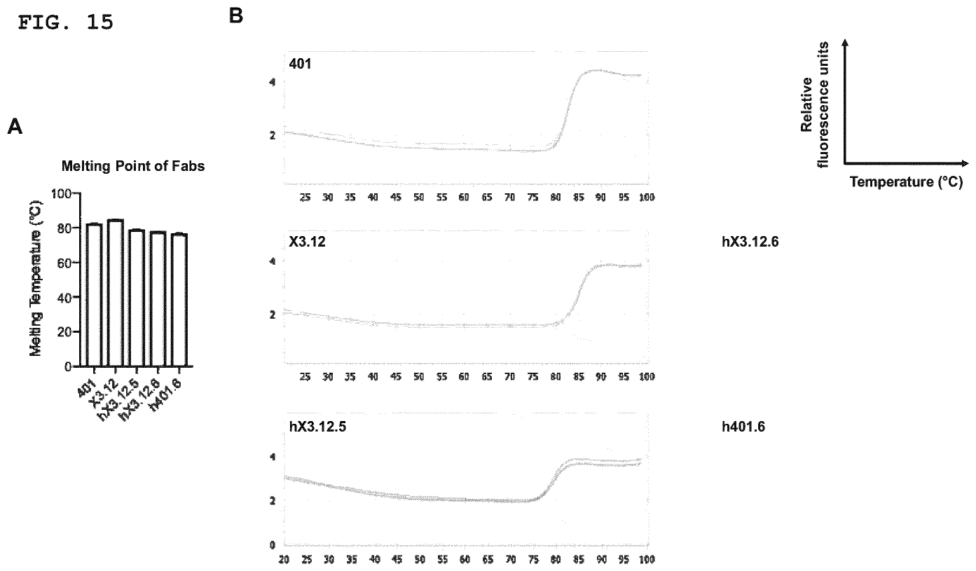

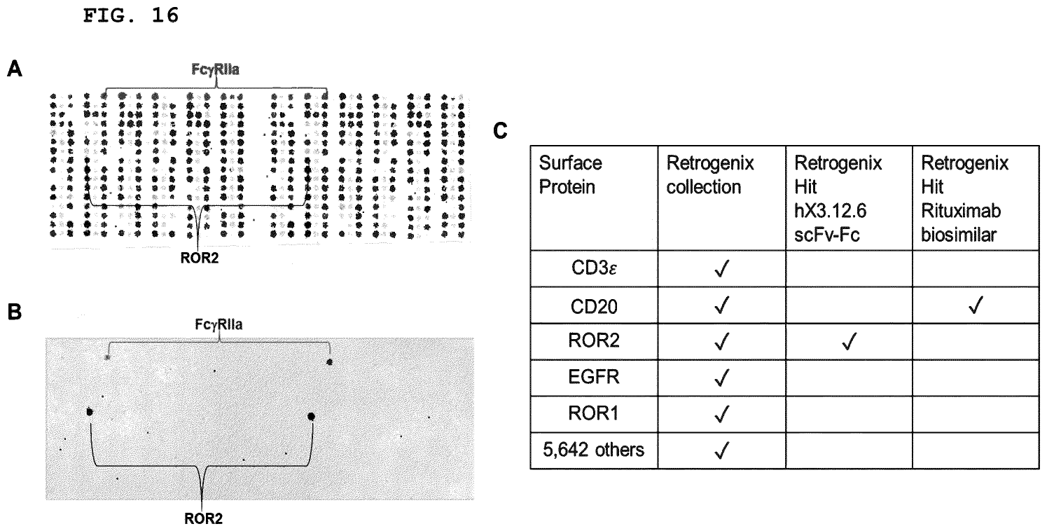

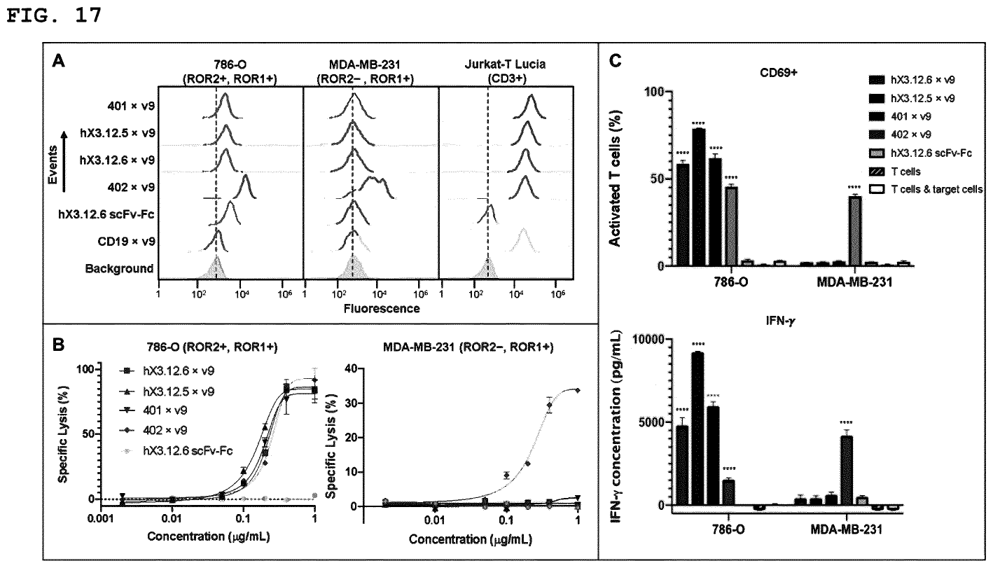

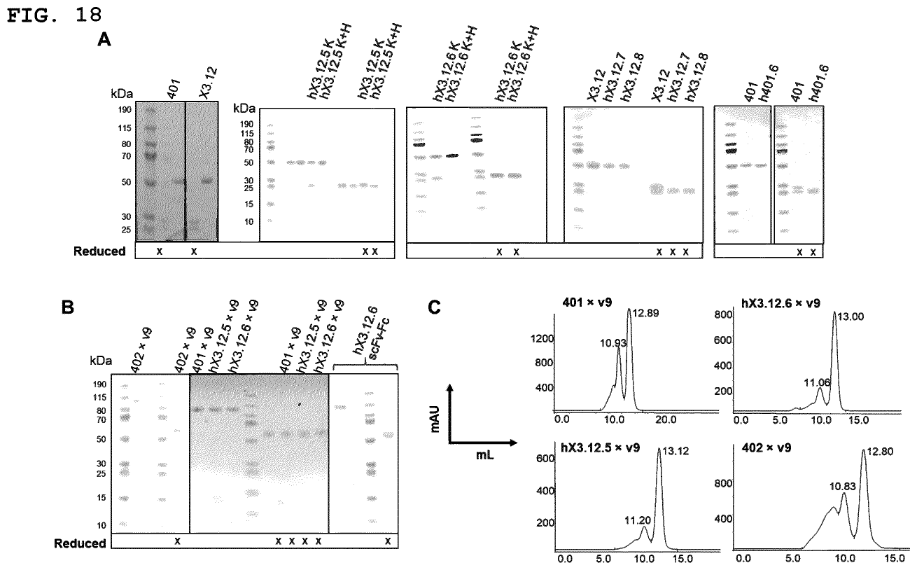

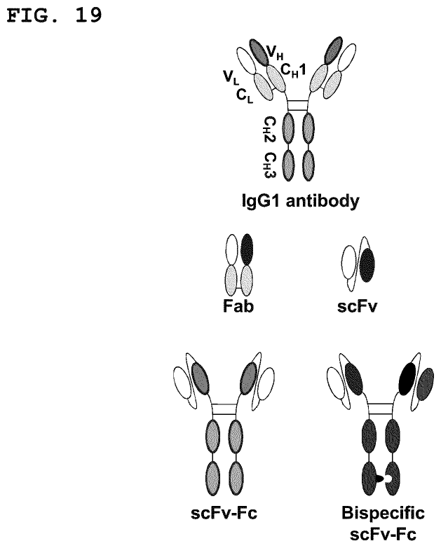

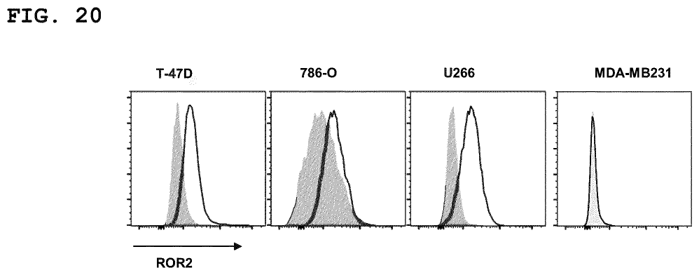

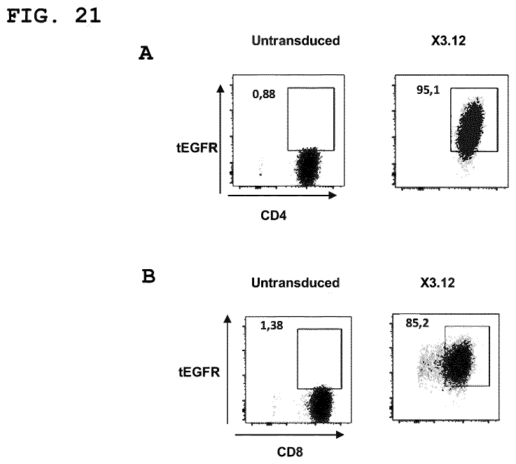

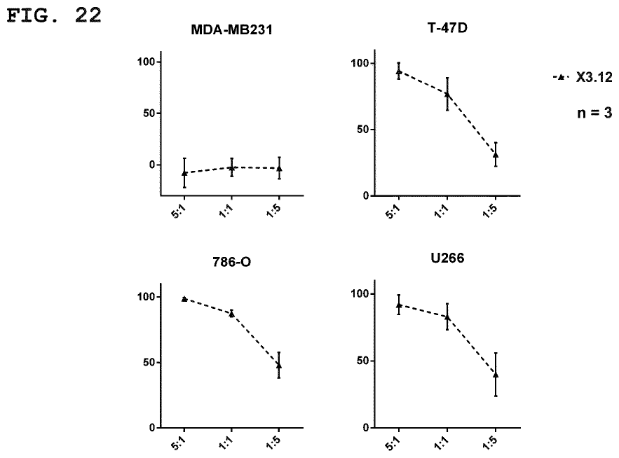

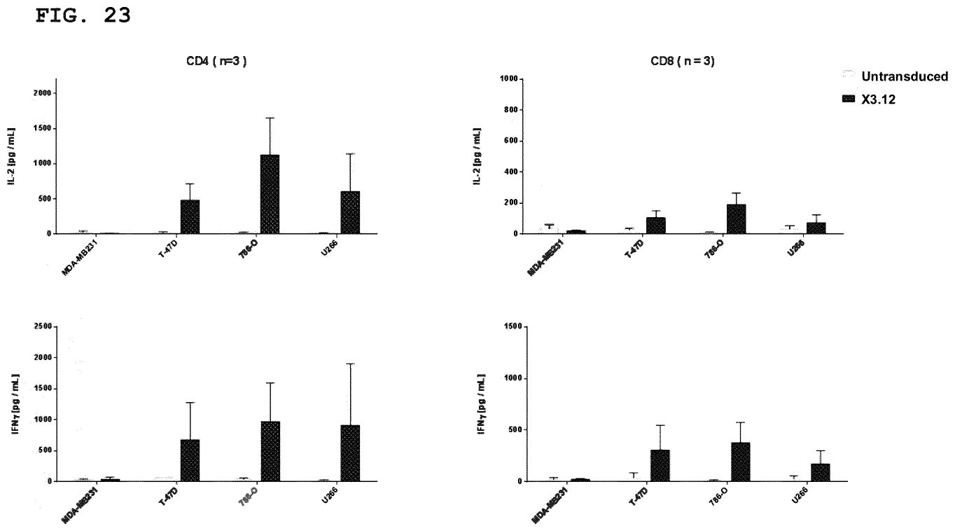

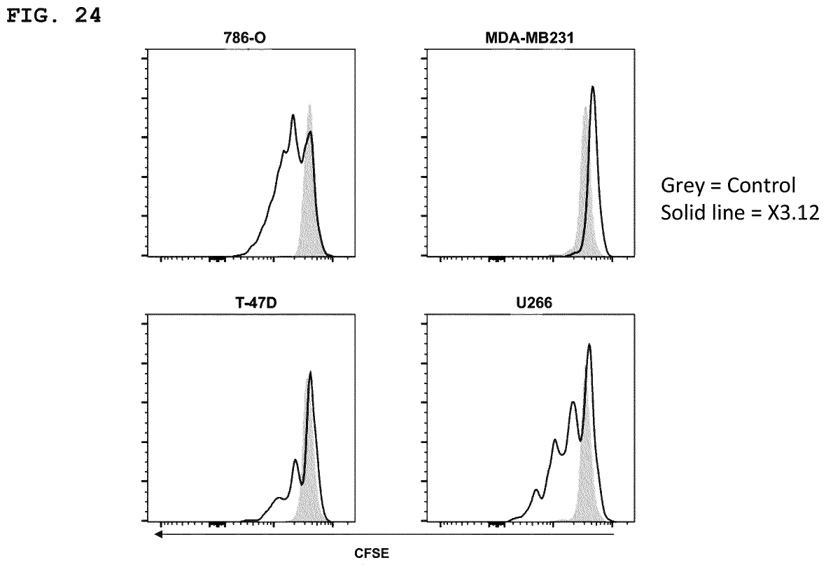

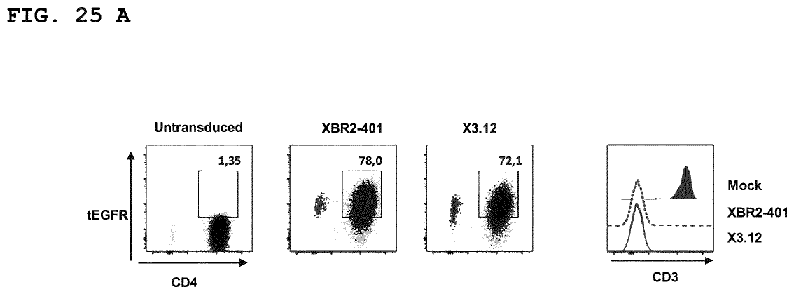

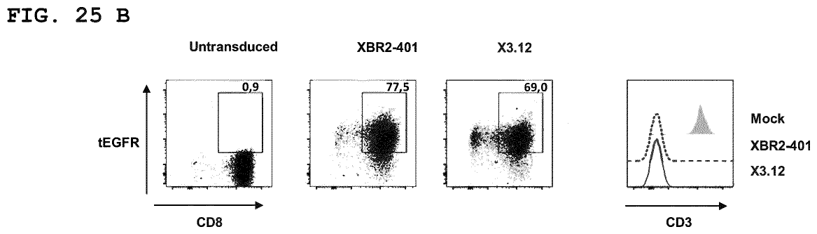

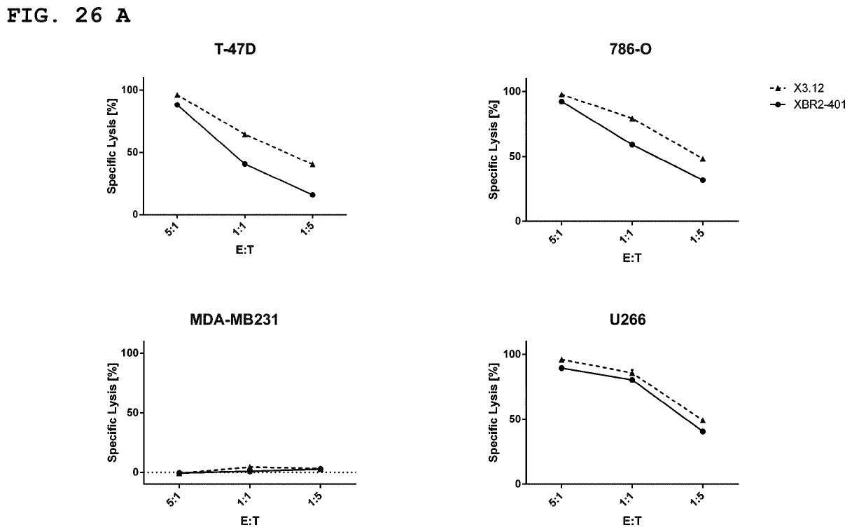

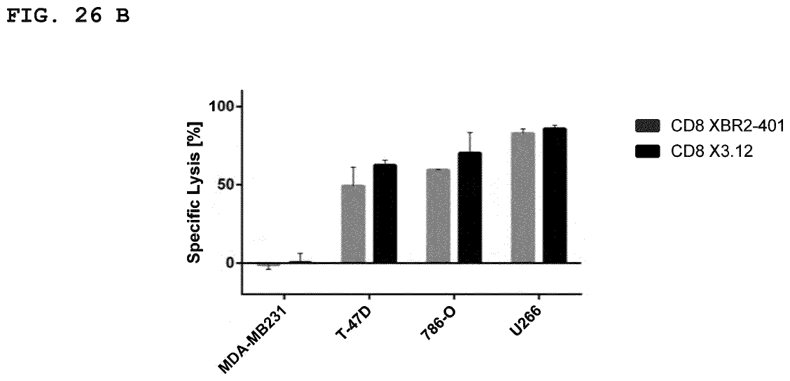

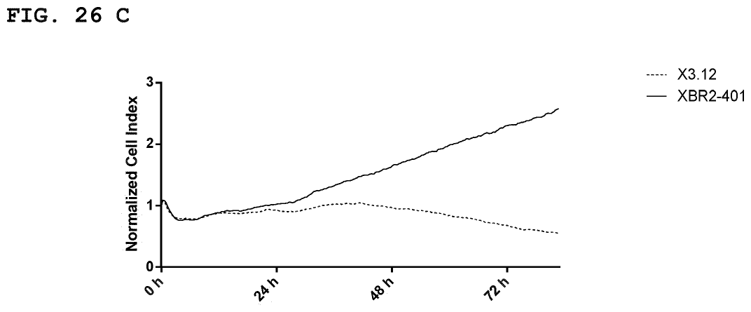

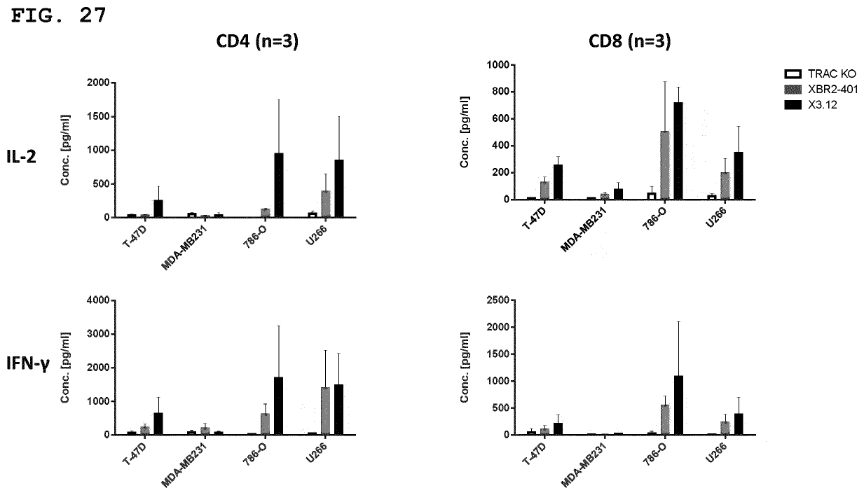

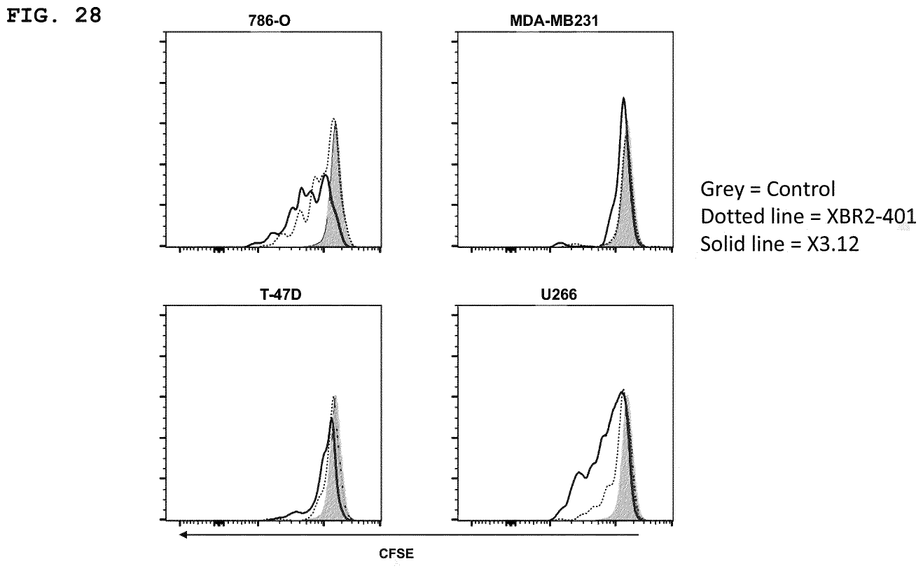

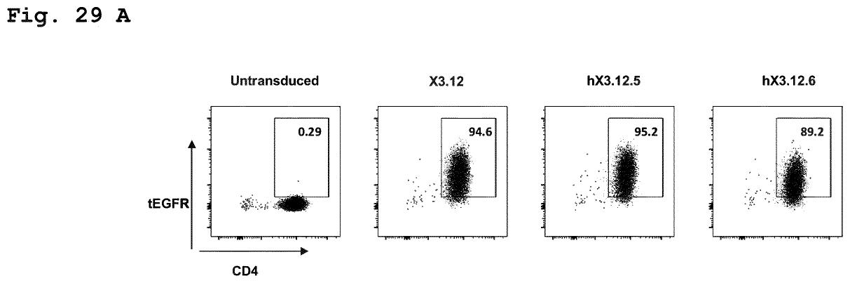

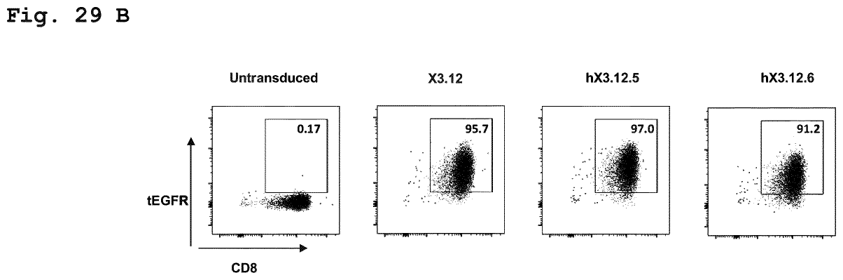

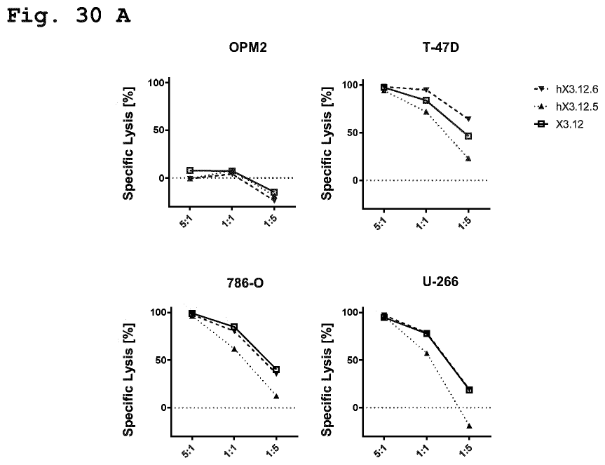

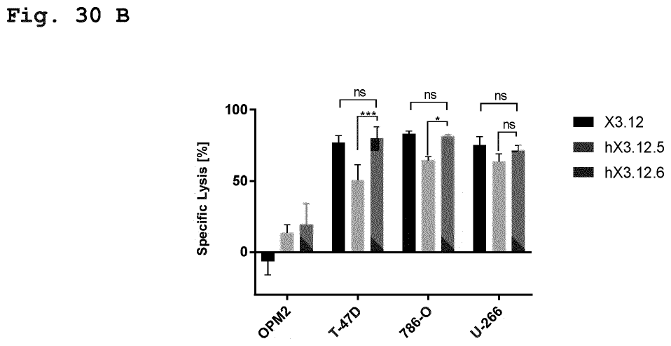

FIG. 14: Analysis of affinity matured and humanized mAbs by SPR. (A) Shown are Biacore X100 sensorgrams of the top 12 chimeric rabbit/human anti-human ROR2 Fabs selected from the focused mutagenesis library by phage display. (B) Shown are Biacore X100 sensorgrams of the humanized anti-human ROR2 Fabs. A CM5 chip immobilized with a mouse anti-human Fcγ mAb was used to capture Fc-hROR2. Fabs were injected at five different concentrations (200, 100, 50, 25, and 12.5 nM for chimeric Fabs and starting at 100 nM for humanized Fabs). Kinetic (kₒₙ and k_{off}) and thermodynamic (K_{D}=k_{off}/kₒₙ) parameters of 1:1 binding were calculated and compiled in Tables III and IV, which are shown in FIGS. 10 and 12 respectively.

FIG. 15. Melting temperature and curves of humanized Fabs. (A) The melting temperature of the indicated Fabs. Error bars represent standard deviations of the average of triplicates (mean±SD) (B) Melting temperatures were determined using a LightCycler 480 protein melting protocol. Curves of triplicates of each Fab are shown. Melting of Fabs was measured up to 99° C.

FIG. 16: Analysis of the specificity of affinity matured and humanized mAb hX3.12.6 by cell microarray technology.

Using Retrogenix′ custom cell microarray technology, mAb hX3.12.6 in scFv-Fc format was screened against 5647 human plasma membrane proteins expressed on the surface of human HEK 293 cells.

(A) Image of Retrogenix′ custom cell microarray ZsGreen1 spotting pattern for approximately 300 human plasma membrane proteins. Microarray also included untransfected HEK293F as a control.

(B) Humanized scFv-Fc xh3.12.6 was screened at 20 μg/mL and was visualized using AlexaFluor 647-conjugated goat anti-human IgG Fcγ pAbs. FcγRIIa is an expected nonspecific hit due to Fc binding

(C) Summary of post-screen where 20 μg/mL hX3.12.6 scFv-Fc against 5647 human antigens arrayed in duplicate revealed ROR2 as the only specific hit where a rituximab biosimilar (1 μg/mL) was screened as control.

FIG. 17: Activity of ROR2×CD3 biAbs.

(A) The indicated ROR2×CD3 biAbs along with control ROR1×CD3 and CD19×CD3 biAbs (all in heterodimeric scFv-Fc format) in conjunction with Alexa Fluor 647-conjugated donkey anti-human F(ab′)2 pAbs were used to stain 786-O, MDA-MB-231, and Jurkat-T Lucia cells to confirm specific binding to ROR2 and CD3. Secondary antibody alone (“background”) served as negative control.

(B) A panel of ROR2×CD3 biAbs based on the humanized and affinity matured mAbs and the parental mAb was compared to a ROR1×CD3 biAb (all in heterodimeric scFv-Fc format) and a monospecific negative control without T-cell engaging arm. Plotted is the specific lysis of cell lines 786-O (ROR2+, ROR1+) and MDA-MB-231 (ROR2+, ROR1−) after 16-h incubation with the indicated range of biAb concentrations and ex vivo expanded T cells at an effector:target cell ratio of 10:1

(C) T-cell activation was measured as percentage of CD69+ T cells determined by flow cytometry and cytokine IFN-γ release determined by ELISA. Based on independent triplicates shown as mean±SD, one-way ANOVA was used to analyze significant differences between ROR2×CD3 (or ROR1×CD3) biAbs and the monospecific scFv-Fc negative control (****, p<0.0001)

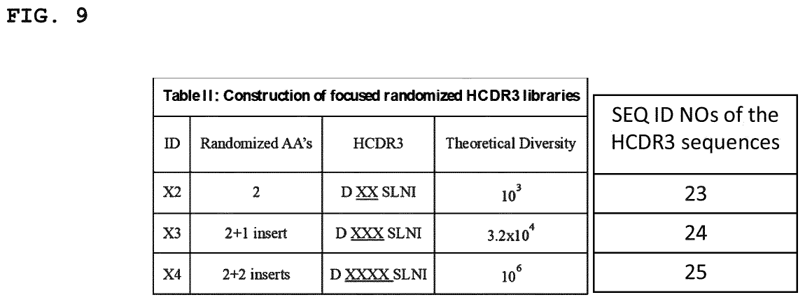

FIG. 18: Purification of affinity matured and humanized Fabs and purification of ROR2×CD3 biAbs. (A) Following tandem purification on KappaSelect and IMAC columns, the indicated Fabs were analyzed by SDS-PAGE and Coomassie blue staining. Expected bands were seen under nonreducing (˜50 kDa) and reducing (˜25 kDa) conditions. (B) Protein A-purified biAbs in heterodimeric scFv-Fc format (and a monospecific scFv-Fc negative control) were confirmed by SDS-PAGE and Coomassie blue staining showing the expected nonreduced bands at ˜100 kDa and reduced bands ˜50 kDa. (C) SEC elution profile of Protein A-purified biAbs. The major peak at ˜13 mL is the monodisperse biAb while the minor peak at ˜11 mL contains higher molecular weight aggregates. The activity assays shown in FIG. 9 are based on tandem Protein A and SEC purification of biAbs.

FIG. 19: From top to bottom: The IgG1 format is naturally found as a dimer containing constant regions (gray) and 2 N-terminal variable regions (V_{H} and V_{L}) which bind the antigen. Fab only consists of the V_{H}, C_{H}1, V_{L}, and C_{L} domains. The scFv-Fc format contains an Fc domain but without C_{L} and C_{H}1 domains. V_{H} and V_{L} are fused via a polypeptide linker. Both variable regions are identical on scFv-Fc. The bispecific scFv-Fc format contains an Fc with knobs-into-holes mutations to allow for a dimer to form between 2 different chains, enabling the combination of 2 different variable regions. The monospecific scFv-Fc format serves as a control for the bispecific scFv-Fc format.

FIG. 20: ROR2 expression of cancer cell lines by flow cytometry.

hX3.12.5×h38C2 DVD antibody was covalently conjugated with AF647 and used to analyze the ROR2 expression pattern on a variety of cancer cell lines, including T-47D (breast cancer), 786-O (renal cell carcinoma), U266 (multiple myeloma) and MDA-MB231 (breast cancer).

FIG. 21: Enrichment and detection of CAR-T cells using the EGFRt transduction marker

Human CD4+ or CD8+ T cells were transduced with a lentiviral vectors encoding the X3.12 CAR and subsequently enriched for CAR expressing cells by magnetic activated cell sorting (MACS) making use of the truncated epidermal growth factor receptor (EGFRt) transduction marker. The coding sequence (CDS) for EGFRt is linked to the CAR CDS by a 2A ribosomal skipping sequence and expression of EGFRt can be used as a surrogate marker for CAR expression.

(A) Flow cytometry plots demonstrating the frequency of EGFRt-positive CD4+ T cells after EGFRt enrichment by MACS.

(B) Flow cytometry plots demonstrating the frequency of EGFRt-positive CD8+ T cells after EGFRt enrichment by MACS.

FIG. 22: Cytolytic activity of X3.12 CAR-expressing CD8+ T cells

Cytolytic activity of primary human CD8+ T cells expressing the affinity matured X3.12 ROR2-specific CAR against ROR2-positive target cells. MDA-MB-231 is a ROR2-negative human breast cancer cell line that was used as a negative control.

T-47D (breast cancer), 786-O (renal cell carcinoma) and U266 (multiple myeloma) are established cancer cell lines that endogenously express ROR2. All target cell lines were engineered to stably express a firefly (P. pyralis) luciferase. The specific lysis of the target cells was calculated based on the intensity of the luminescence signal after addition of Luciferin to a final concentration of 150 μg/ml.

FIG. 23: Cytokine secretion of X3.12-based ROR2-specific CAR-expressing T cells

CD4+ or CD8+ CAR-T cells expressing the X3.12 CAR were co-cultured with ROR2-positive target cells at an E:T ratio of 4:1. Concentrations of the effector cytokines IL-2 and IFN-γ were measured by ELISA in the cell culture supernatants after 24 h co-culture. Comparative cytokine secretion is shown for ROR2-positive target cells (T-47D, 786-O and U266), as well as MDA-MB-231 for CD4+ and CD8+ T cells from n=3 independent donors.

FIG. 24: Proliferation of X3.12 CAR-expressing T cells

Proliferation of CD4+ ROR2-specific CAR-T cells after stimulation with irradiated ROR2-positive target cells at an E:T ratio of 4:1. No exogenous cytokines were added to the culture media and the T cell proliferation was assessed by CFSE dye dilution 72 h after stimulation.

CFSE flow cytometry histograms of X3.12 ROR2-specific CAR-T cells against ROR2-positive (T-47D, 786-0 and U266), or ROR2-negative target cells. Grey filled curves represent control T cells (untransduced).

FIG. 25: Enrichment and detection of CAR knock-in T cells using the EGFRt transduction marker

Human CD4+ or CD8+ T cells were co-electroporated with hTRAC-specific sgRNA/spCas9 ribonucleoprotein (RNP) and a homology dependent repair template (HDRT) containing the XBR2-401 or X3.12 CAR coding cassettes. CAR knock-in T cells were enriched by magnetic activated cell sorting (MACS) making use of the truncated epidermal growth factor receptor (EGFRt) transduction marker. The coding sequence (CDS) for EGFRt is linked to the CAR CDS by a 2A ribosomal skipping sequence and expression of EGFRt can be used as a surrogate marker for CAR expression. The T cell receptor and CD3 are co-shuttled to the cell surface. Thus, CD3 negativity can be used to detect TRAC-KO and knock-in T cells.

(A) Flow cytometry plots demonstrating the frequency of EGFRt-positive CD4+ T cells after EGFRt enrichment by MACS. CD3 negativity of CAR knock-in T cells is represented in the adjacent histogram.

(B) Flow cytometry plots demonstrating the frequency of EGFRt-positive CD8+ T cells after EGFRt enrichment by MACS. CD3 negativity of CAR knock-in T cells is represented in the adjacent histogram.

FIG. 26: Cytolytic activity of ROR2 CAR knock-in CD8+ T cells

(A) Cytolytic activity of primary human CD8+ T cells expressing either the XBR2-401 or the affinity matured X3.12 CAR under control of the T cell receptor locus promotor against ROR2-positive target cells. MDA-MB-231 is a ROR2-negative human breast cancer cell line that was used as a negative control. T-47D (breast cancer), 786-O (renal cell carcinoma) and U266 (multiple myeloma) are established cancer cell lines that endogenously express ROR2. All target cell lines were engineered to stably express a firefly (P. pyralis) luciferase. The specific lysis of the target cells was calculated based on the intensity of the luminescence signal after addition of Luciferin to a final concentration of 150 μg/ml. (Data is a representative of n=3 independent donors)

(B) Combined data for the antigen-dependent cytolytic activity of ROR2-specific CAR-T cells against a variety of ROR2-positive target cells (T-47D, 786-O, U266). ROR2-negative MDA-MB231 cells were used as a negative control. (E:T ratio=1:1, t=24 h, n=3)

(C) Cytolytic activity of primary human CD8+ T cells expressing either the XBR2-401 or the X3.12 ROR2-specific CAR under control of the endogenous T cell receptor promotor against ROR2-positive 786-O cells, using the xCELLigence platform. The normalized cell index, which directly correlates with the quantity of living, adherent tumor cells, was measured and plotted over time. (Data is a representative of n=2 independent donors).

FIG. 27: Cytokine secretion of XBR2-401 or X3.12-based ROR2-specific CAR knock-in T cells

CD4+ or CD8+ CAR-T cells expressing either the XBR2-401 or X3.12 CAR were co-cultured with ROR2-positive target cells at an E:T ratio of 4:1. Concentrations of the effector cytokines IL-2 and IFN-γ were measured by ELISA in the cell culture supernatants after 24 h co-culture. Comparative cytokine secretion is shown for ROR2-positive target cells (T-47D, 786-O and U266), as well as MDA-MB-231 for CD4+ and CD8+ T cells from n=3 independent donors.

FIG. 28: Proliferation of XBR2-401 or X3.12-based ROR2-specific CAR knock-in T cells

Proliferation of CD8+ROR2-specific CAR-T cells after stimulation with irradiated ROR2-positive target cells at an E:T ratio of 4:1. No exogenous cytokines were added to the culture media and the T cell proliferation was assessed by CFSE dye dilution 72 h after stimulation. CFSE flow cytometry histograms of XBR2-401 (dotted line) or X3.12 (solid line) ROR2-specific CAR-T cells against ROR2-positive (T-47D, 786-O and U266), or ROR2-negative target cells. Grey filled curves represent medium controls for the XBR2-401 CAR.

FIG. 29: Generation, detection and enrichment of ROR2 CAR-T cells

Human CD4+ or CD8+ T cells were transduced with a lentiviral vector encoding the X3.12, hX3.12.5 or hX3.12.6 CAR and subsequently enriched for CAR expressing cells by magnetic activated cell sorting (MACS) making use of the truncated epidermal growth factor receptor (EGFRt) transduction marker. The coding sequence (CDS) for EGFRt is linked to the CAR CDS by a 2A ribosomal skipping sequence and expression of EGFRt can be used as a surrogate marker for CAR expression.

(A) Flow cytometry plots demonstrating the frequency of EGFRt-positive CD4+ T cells after EGFRt enrichment by MACS.

(B) Flow cytometry plots demonstrating the frequency of EGFRt-positive CD8+ T cells after EGFRt enrichment by MACS.

FIG. 30: Cytolytic activity of humanized ROR2 CAR-T cells

(A) Cytolytic activity of primary human CD8+ T cells expressing a X3.12, hX3.12.5 or hX3.12.6-based CAR against ROR2-positive target cells. OPM-2 is a ROR2-negative human multiple myeloma cell line that was used as a negative control. T-47D (breast cancer), 786-O (renal cell carcinoma) and U266 (multiple myeloma) are established cancer cell lines that endogenously express ROR2. All target cell lines were engineered to stably express a firefly (P. pyralis) luciferase. The specific lysis of the target cells was calculated based on the intensity of the luminescence signal after addition of Luciferin to a final concentration of 150 μg/ml. (Data is a representative of n=3 independent donors)

(B) Combined data for the antigen-dependent cytolytic activity of X3.12, hX3.12.5 or hX3.12.6-based CAR-T cells against a variety of ROR2-positive target cells (T-47D, 786-O, U266). ROR2-negative OPM-2 cells were used as a negative control. (E:T ratio=5:1, t=6 h, n=3). Statistics are based on repeated-measures ANOVA and pairwise comparison by Dunnet's multiple comparisons test (n.s. not significant, *=p<0.05, **=p<0.01, ***, p<0.001)

FIG. 31: Flow cytometric analysis of ROR2 expression on OPM-2 cell line.

hX3.12.6×h38C2 DVD antibody was covalently conjugated with AF647 and used to analyze the ROR2 expression pattern on a variety of OPM-2 cells (multiple myeloma).

FIG. 32: Cytokine secretion of X3.12, hX3.12.5 and hX3.12.6-based ROR2-specific CART cells

CD4+ or CD8+ CAR-T cells expressing the X3.12, hX3.12.5 or hX3.12.6-based CAR were co-cultured with ROR2-positive target cells at an E:T ratio of 4:1. Concentrations of the effector cytokine IFN-γ were measured by ELISA in the cell culture supernatants after 24 h co-culture. Comparative cytokine secretion is shown for ROR2-positive target cells (T-47D, 786-O and U266), as well as MDA-MB-231 for CD4+ and CD8+ T cells from n=3 independent donors. Statistics are based on repeated-measures ANOVA and pairwise comparison by Dunnet's multiple comparisons test (n.s. not significant, *=p<0.05, **=p<0.01, ***, p<0.001)

FIG. 33: Proliferation of ROR2 CAR-T cells

Antigen-dependent proliferation of CD4+ and CD8+ X3.12, hX3.12.5 or hX3.12.6-based ROR2-specific CAR-T cells after stimulation with irradiated ROR2-positive or ROR2-negative target cells at an E:T ratio of 4:1. No exogenous cytokines were added to the culture media and the T cell proliferation was assessed by CFSE dye dilution 72 h after stimulation. The fraction of cells that underwent at least one cell division was calculated as a measure to quantify antigen-dependent proliferation. (n=3 independent donors). Statistics are based on repeated-measures ANOVA and pairwise comparison by Dunnet's multiple comparisons test (n.s. not significant, *=p<0.05, **=p<0.01, ***, p<0.001).

### DETAILED DESCRIPTION OF THE INVENTION

Unless specifically defined herein, all technical and scientific terms used herein have the same meaning as commonly understood by a skilled artisan in the fields of gene therapy, immunology, biochemistry, genetics, and molecular biology.

All methods and materials similar or equivalent to those described herein can be used in the practice or testing of the present invention, with suitable methods and materials being described herein. All publications, patents and patent applications cited herein are hereby incorporated by reference in their entirety for all purposes. References referred to herein are indicated by a reference number in square brackets (e.g. as “[31]” or as “reference [31]”), which refers to the respective reference in the list of references at the end of the description. In case of conflict, the present specification, including definitions, will prevail over the cited references. Further, the materials, methods, and examples are illustrative only and are not intended to be limiting, unless otherwise specified.

Antibodies of non-human origin can be humanized by CDR grafting by methods known in the art. The humanization increases the homology of the binding domains to human antibody binding domains (i.e. the humanness), and reduces the immunogenic potential of the humanized antibody in human beings, which in turn is expected to increase the safety and therapeutic application profile in human patients. On the other hand, antibody humanization is often accompanied by a reduction of the binding affinity of the humanized antibody to its antigen, often requiring affinity maturation, see reference [31], which is hereby incorporated by reference in its entirety for all purposes.

It has also been experienced that the use of humanized antibody fragments for the generation of a targeting domain of a CAR can result in a lower performance of the CAR with respect to binding to the target antigen and triggering effector functions of the CAR expressing cell.

In the present invention, the inventors deployed rational design-based affinity maturation of an existing anti-ROR2 antibody (rabbit, WO 2017/127702 A1, which is hereby incorporated by reference in its entirety for all purposes), using unpublished protein crystal structure data concerning epitope and paratope. Novel binders were isolated from phage display libraries and characterized in vitro. Surface plasmon resonance revealed a 10-fold increase in binding affinity (Kd=0.7 nM).

The most potent clones were selected and antibody humanization was performed. The affinity matured, humanized clones (hX3.12.5 and hX3.12.6) were again validated in vitro, showing a slight decrease in binding affinity (Kd=2.6 nM and Kd=3.8 nM, respectively), as compared to the affinity matured clone (X3.12). Cell microarray technology was used to validate antigen-specificity, Additionally, these findings were confirmed by protein X-ray crystal structure analysis.

The inventors generated ROR2×CD3 bi-mAbs for the matured, humanized clones, as well as the parental antibody. All clones showed potent antigen-specific anti-tumor efficacy, as determined by cell cytolysis assays. Additionally, the hX3.12.5×CD3 bi-mAb showed significantly more potent T cell activation, as determined by CD69 expression levels and IFN-γ secretion.

The inventors also generated 2^{nd} generation ROR2 CAR-T cells using the parental and the affinity matured antibody clones and performed functional characterization in vitro. In brief, both antibody versions show potent anti-tumor efficacy, as determined by antigen-dependent cytolysis, cytokine secretion and proliferation. Under dose-limiting conditions, the CAR derived from the affinity matured antibody outperformed the parental clone.

A “recombinant mammalian cell” according to the invention can be any cell as defined herein. Preferably, a recombinant mammalian cell is an isolated cell. Recombinant mammalian cells according to the invention can be produced in accordance with known pharmaceutical standards. For instance, they can be formulated for administration to humans.

A “CAR” according to the invention can be any possible form. In a preferred embodiment, the CAR is present in an isolated form. In another preferred embodiment the CAR according to the invention or a nucleic acid encoding the CAR can be present in a composition. The composition may be a pharmaceutical composition.

The invention includes switchable CAR T-cells that are controlled by a Fab or other antibody fragment. Examples of such types of switchable CAR T-cells are described in [64-67]. This can be achieved by linking an adaptor, a molecule bound by a universal CAR, to a Fab or other antibody fragment that recognizes ROR2. Such switchable CAR-T cells can only target and kill ROR2-expressing cancer cells when the adaptor is administered. This allows for titratable and reversible control of the CAR T-cells, as exemplified in [67]. Switchable CAR-T platforms also permit administration of different adaptors simultaneously or sequentially. Different adaptors may target the same or different antigens, e.g., ROR2 and an additional antigen. The use of multiple adaptors could effectively evolve CAR-T therapy from monoclonal to polyclonal recognition to counteract target cell heterogeneity and resistance in cancer. Thus, in a preferred embodiment of the invention, an antibody derivative capable of binding to human ROR2 according to the invention can be a derivative which comprises an adaptor for a universal CAR. It will be understood by a person skilled in the art that such a derivative is capable of conferring ROR2-specificity to the universal CAR.

Sequence alignments of sequences according to the invention are performed by suitable algorithms, and preferably by using the BLAST algorithm, see references [32, 33], using suitable alignment parameters as known in the art.

The terms “K_{D}” or “K_{D} value” relate to the equilibrium dissociation constant as known in the art. In the context of the present invention, these terms can relate to the equilibrium dissociation constant of an antibody or derivative thereof capable of binding to ROR2 (e.g. a CAR T-cell or an antibody) with respect to the antigen of interest (i.e. ROR2). The equilibrium dissociation constant is a measure of the propensity of a complex (e.g. an antigen-targeting agent complex) to reversibly dissociate into its components (e.g. the antigen and the targeting agent). Methods to determine K_{D} values are known in art. Preferably, K_{D} values are determined by surface plasmon resonance measurements.

The term “antibody” as used herein refers to any functional antibody that is capable of specific binding to the antigen of interest. Without particular limitation, the term antibody encompasses antibodies from any appropriate source species, including avian such as chicken and mammalian such as mouse, goat, non-human primate and human. Preferably, the antibody is a humanized or human antibody. Humanized antibodies are antibodies which contain human sequences and a minor portion of non-human sequences which confer binding specificity to an antigen of interest (e.g. ROR2). The antibody is preferably a monoclonal antibody which can be prepared by methods well-known in the art. The term antibody encompasses an IgG-1, -2, -3, or -4, IgE, IgA, IgM, or IgD isotype antibody. The term antibody encompasses monomeric antibodies (such as IgD, IgE, IgG) or oligomeric antibodies (such as IgA or IgM). The term antibody also encompasses—without particular limitations—isolated antibodies and modified antibodies such as genetically engineered antibodies, e.g. chimeric antibodies or bispecific antibodies, or antibody conjugates with a drug such as an anticancer drug or a cytotoxic drug. A bispecific antibody capable of binding to ROR2 in accordance with the invention can be a T-cell engager such as a CD3×ROR2 BiTE (Bi-specific T-cell engager) or a DART (dual-affinity re-targeting proteins). An “antibody” (e.g. a monoclonal antibody) or “a derivative thereof” as described herein may have been linked to a different molecule. For example, molecules that may be linked to the antibody are other proteins (e.g. other antibodies), a molecular label (e.g. a fluorescent, luminescent, colored or radioactive molecule), a pharmaceutical and/or a toxic agent. The antibody or antigen-binding portion may be linked directly (e.g. in form of a fusion between two proteins), or via a linker molecule (e.g. any suitable type of chemical linker known in the art).

An antibody derivative or derivative of an antibody capable of binding to ROR2 as used herein comprises a portion of an antibody that retains the capability of the antibody to specifically bind to the ROR2 antigen. This capability can, for instance, be determined by determining the capability of the antigen-binding portion to compete with the antibody for specific binding to the antigen by methods known in the art. Without particular limitation, the antibody derivative can be produced by any suitable method known in the art, including recombinant DNA methods and preparation by chemical or enzymatic fragmentation of antibodies. Antibody derivatives may be fragments may be Fab fragments, F(ab′) fragments, F(ab′)2 fragments, single chain antibodies (scFv), single-domain antibodies, diabodies or any other portion(s) of the antibody that retain the capability of the antibody to specifically bind to the antigen. Antibody derivatives of the invention may also be chimeric antigen receptors (CARs).

The nomenclature of antibodies or derivatives thereof including their variable domains and their CDR sequences, as used in connection with the present invention, follows the Kabat numbering. The Kabat numbering is described in Kabat E. A., Wu, T. T., Perry, H., Gottesman, K. and Foeller, C. (1991) Sequences of Proteins of Immunological Interest, Fifth Edition. NIH Publication No. 91-3242 (reference [68]).

Terms such as “treatment of cancer” or “treating cancer” or “cancer therapy” or “cancer immunotherapy” according to the present invention refer to a therapeutic treatment. An assessment of whether or not a therapeutic treatment works can, for instance, be made by assessing whether the treatment inhibits cancer growth in the treated patient or patients. Preferably, the inhibition is statistically significant as assessed by appropriate statistical tests which are known in the art. Inhibition of cancer growth may be assessed by comparing cancer growth in a group of patients treated in accordance with the present invention to a control group of untreated patients, or by comparing a group of patients that receive a standard cancer treatment of the art plus a treatment according to the invention with a control group of patients that only receive a standard cancer treatment of the art. Such studies for assessing the inhibition of cancer growth are designed in accordance with accepted standards for clinical studies, e.g. double-blinded, randomized studies with sufficient statistical power. The term “treating cancer” includes an inhibition of cancer growth where the cancer growth is inhibited partially (i.e. where the cancer growth in the patient is delayed compared to the control group of patients), an inhibition where the cancer growth is inhibited completely (i.e. where the cancer growth in the patient is stopped), and an inhibition where cancer growth is reversed (i.e. the cancer shrinks). An assessment of whether or not a therapeutic treatment works can be made based on known clinical indicators of cancer progression. In the context of hematological cancers which do not form solid tumors, cancer growth may be assessed by known methods such as methods based on a counting of the cancer cells.

A treatment of cancer according to the present invention does not exclude that additional or secondary therapeutic benefits also occur in patients.

The treatment of cancer according to the invention can be a first-line therapy, a second-line therapy, a third-line therapy, or a fourth-line therapy. The treatment can also be a therapy that is beyond is beyond fourth-line therapy. The meaning of these terms is known in the art and in accordance with the terminology that is commonly used by the US National Cancer Institute.

The term “capable of binding” as used herein refers to the capability to form a complex with a molecule that is to be bound (e.g. ROR2). Binding typically occurs non-covalently by intermolecular forces, such as ionic bonds, hydrogen bonds and Van der Waals forces and is typically reversible. Various methods and assays to determine binding capability are known in the art. Binding is usually a binding with high affinity, wherein the affinity as measured in K_{D} values is preferably less than 1 μM more preferably less than 50 nM, even more preferably less than 10 nM, even more preferably less than 7 nM, even more preferably less than 5 nM, even more preferably less than 1 nM, or in the range of 0.1 nM to 1 nM.

For example, antibodies or derivatives thereof according to the present invention are capable of binding to ROR2, wherein the affinity to ROR2 as measured in K_{D} values is preferably less than 50 nM, more preferably less than 10 nM, even more preferably less than 7 nM, even more preferably less than 5 nM, even more preferably less than 1 nM, or in the range of 0.1 nM to 1 nM. Pharmaceutical compositions in accordance with the present invention are prepared in accordance with known standards for the preparation of pharmaceutical compositions. For instance, the compositions are prepared in a way that they can be stored and administered appropriately. That is, they may, for instance, contain pharmaceutically acceptable components such as carriers, excipients or stabilizers. Such pharmaceutically acceptable components are not toxic in the amounts used when administering the pharmaceutical composition to a patient. The pharmaceutical acceptable components added to the pharmaceutical compositions can be selected based on the chemical nature of the active agents (e.g. the antibody or derivative thereof capable of binding to ROR2, or a recombinant cell according to the invention, or a nucleic acid according to the invention), the particular intended use of the pharmaceutical compositions and the route of administration. It is understood that in accordance with the invention, the compositions are suitable for administration to humans.

A pharmaceutically acceptable carrier can be used herein as known in the art. As used herein, the term “pharmaceutically acceptable” means being approved by a regulatory agency of the Federal or a state government or listed in the U.S. Pharmacopeia, European Pharmacopeia or other generally recognized pharmacopeia for use in mammals, and more particularly in humans. Pharmaceutically acceptable carriers include, but are not limited to, saline, buffered saline, dextrose, water, glycerol, sterile isotonic aqueous buffer, and combinations thereof. It will be understood that the formulation will be appropriately adapted to suit the mode of administration.

A “plasmid” as used in accordance with the invention can be any type of plasmid known in the art which is suitable for use in the invention. For instance, the plasmid can be a nanoplasmid.

As used herein, each occurrence of terms such as “comprising” or “comprises” may optionally be substituted with “consisting of” or “consists of”.

### Further Preferred Embodiments

In a preferred aspect, the invention encompasses an antibody capable of binding to human ROR2 or a derivative thereof capable of binding to human ROR2, wherein the antibody or derivative thereof comprises a light chain variable domain and a heavy chain variable domain, wherein the heavy chain variable domain comprises a CDR3 sequence having an amino acid sequence selected from the group consisting of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 37, and SEQ ID NO: 38.

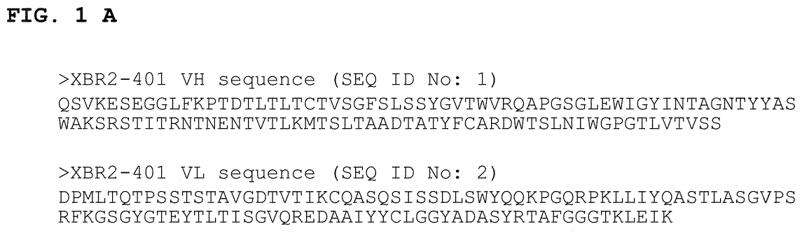

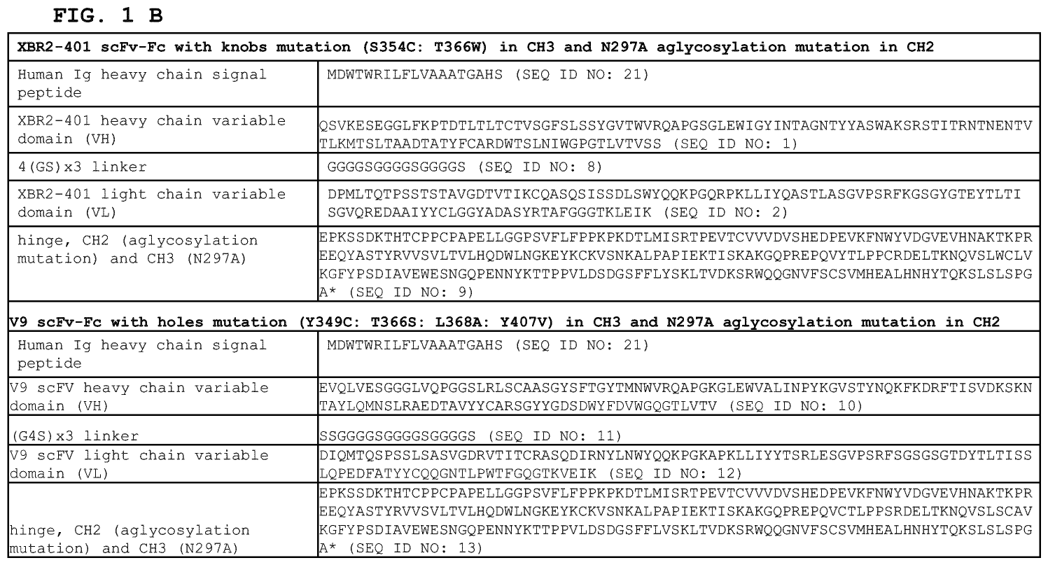

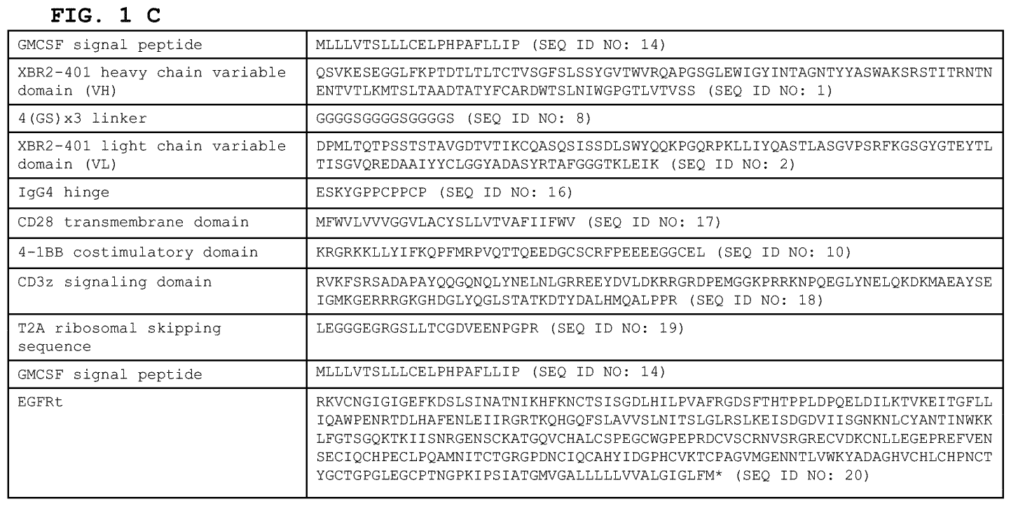

In another aspect, the invention encompasses a bispecific antibody comprising an scFv-Fc with knobs mutation as defined by the amino acid sequences shown in FIG. 1B and comprising an scFv-Fc with holes mutation as defined by the amino acid sequences shown in FIG. 1B.

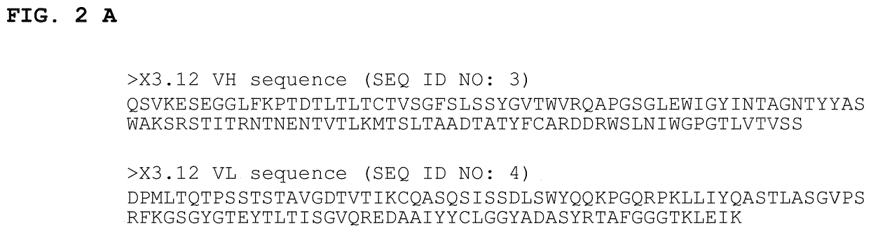

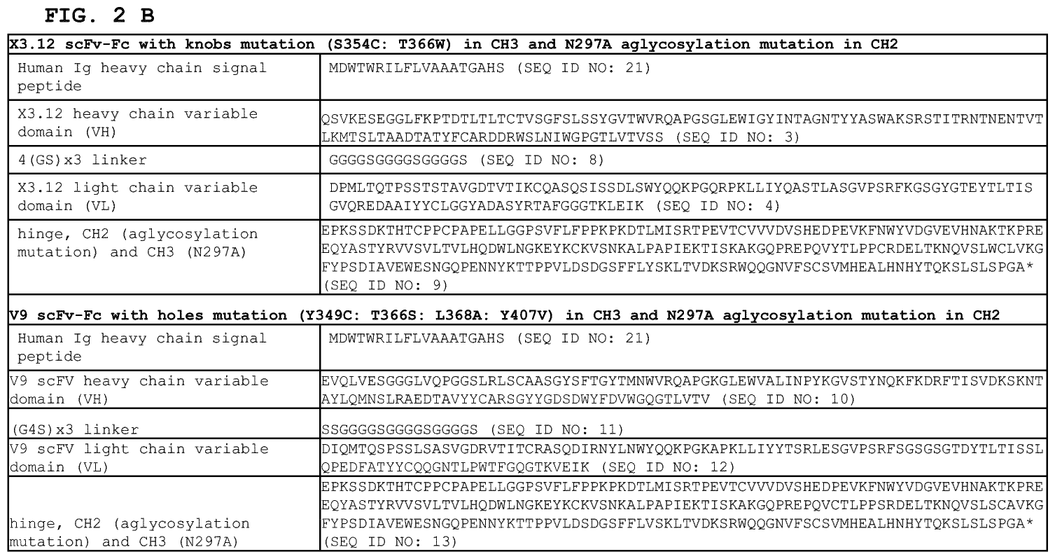

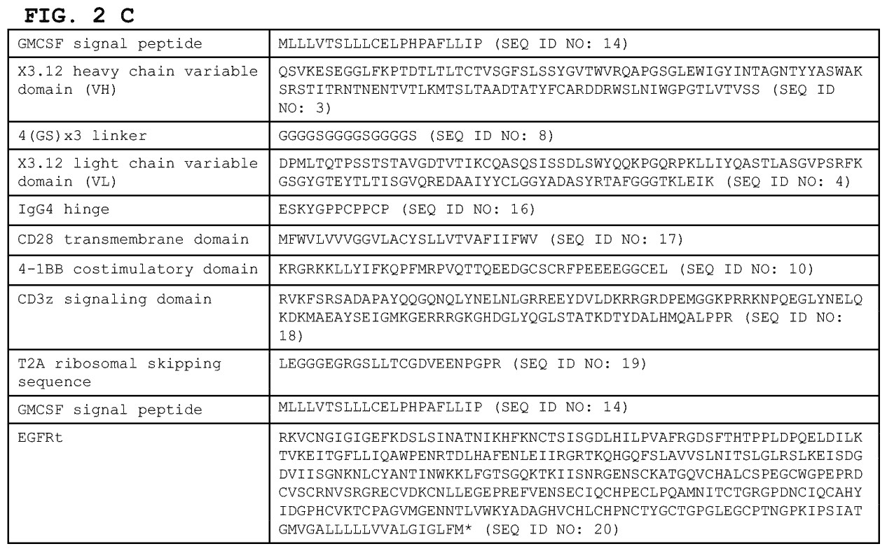

In another preferred aspect, the invention encompasses a bispecific antibody comprising an scFv-Fc with knobs mutation as defined by the amino acid sequences shown in FIG. 2B and comprising an scFv-Fc with holes mutation as defined by the amino acid sequences shown in FIG. 2B.

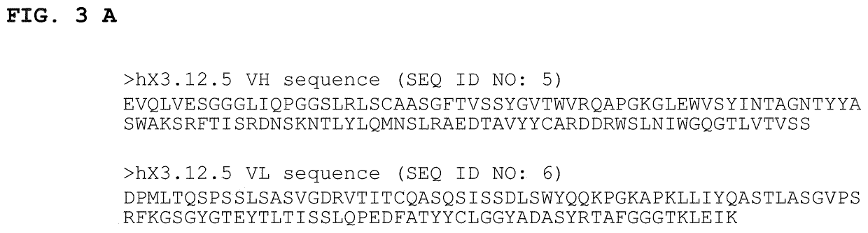

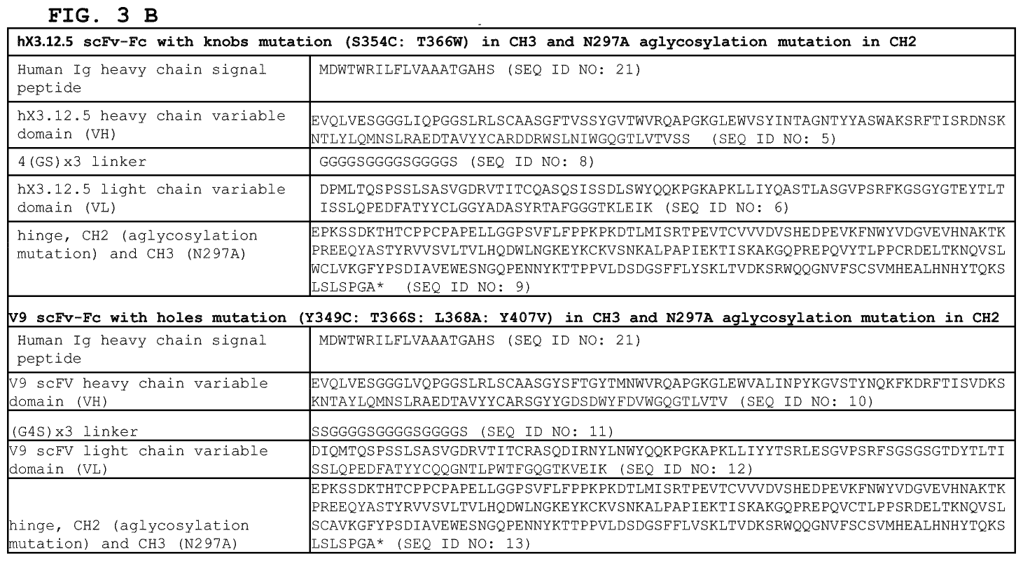

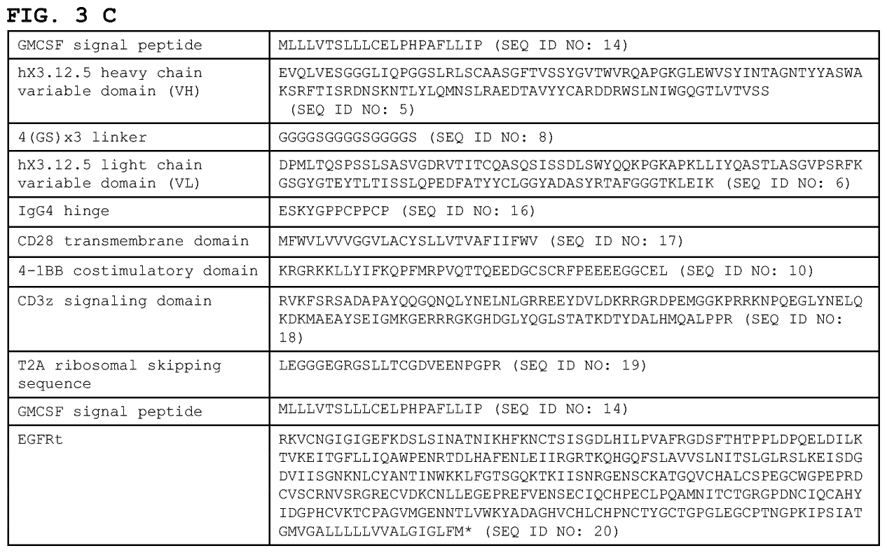

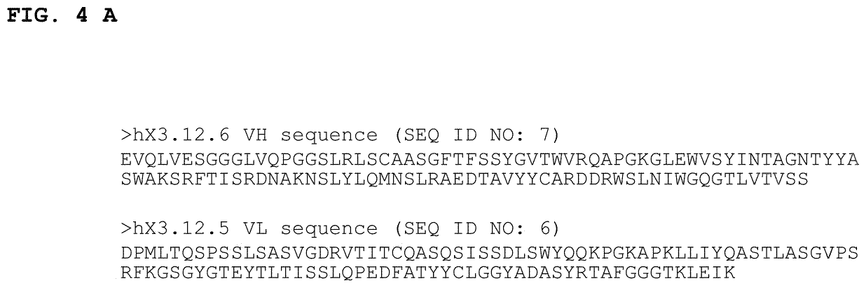

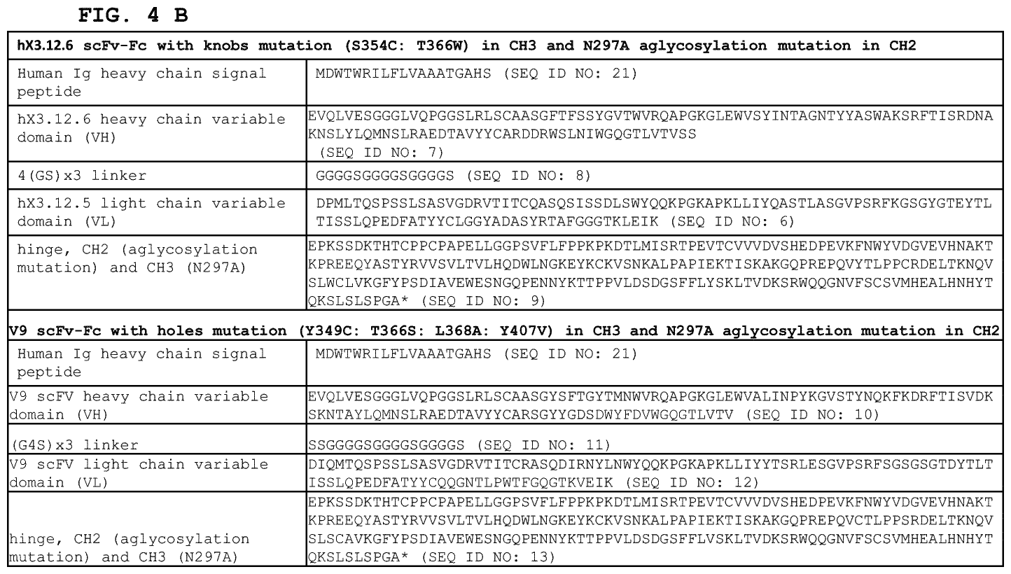

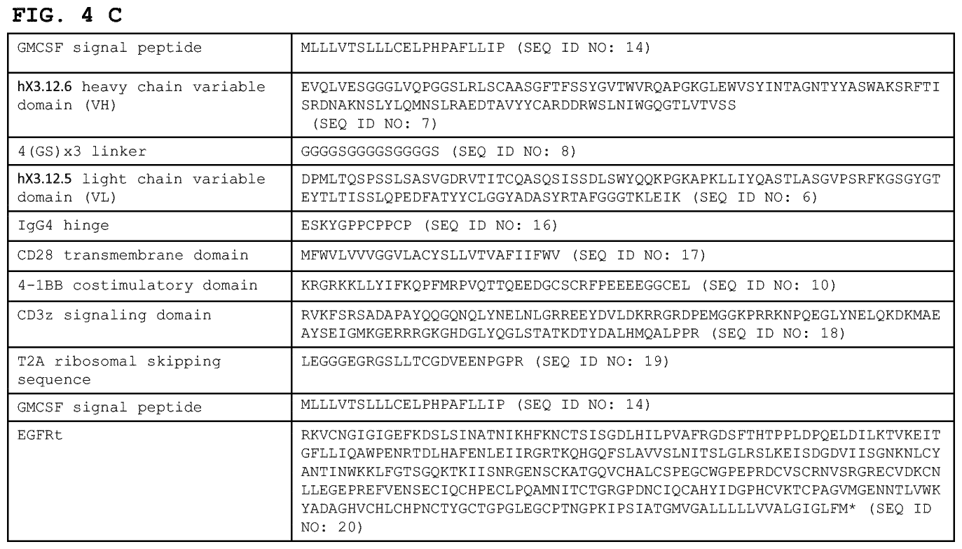

In another preferred aspect, the invention encompasses a bispecific antibody comprising an scFv-Fc with knobs mutation as defined by the amino acid sequences shown in FIG. 3B and comprising an scFv-Fc with holes mutation as defined by the amino acid sequences shown in FIG. 3B.

In another aspect, the invention encompasses a CAR as defined by the amino acid sequences shown in FIG. 1C.

In another preferred aspect, the invention encompasses a CAR as defined by the amino acid sequences shown in FIG. 2C.

In another preferred aspect, the invention encompasses a CAR as defined by the amino acid sequences shown in FIG. 3C.

In accordance with all other embodiments of the invention, the antibodies and derivatives thereof according to the invention are preferably capable of binding to the kringle domain of human ROR2.

In further preferred embodiments of the above aspects and embodiments, the antibody or derivative thereof may have any of the other features of the invention described herein. It may, for instance, be part of a pharmaceutical composition or CAR which can be used in the treatment of the cancers described herein. Likewise, the invention also encompasses nucleic acids encoding the antibody or derivative thereof of the above aspects of the invention, e.g. in the form of vectors such as viral vectors, and pharmaceutical compositions comprising such nucleic acids.

Sequences

The sequences referred to herein are as follows:

Amino acid sequences are indicated using the standard single-letter amino acid code in an N- to C-terminal order, unless indicated otherwise. Nucleic acid sequences are indicated using the standard nucleic acid code in a 5′-to-3′ order, unless indicated otherwise.

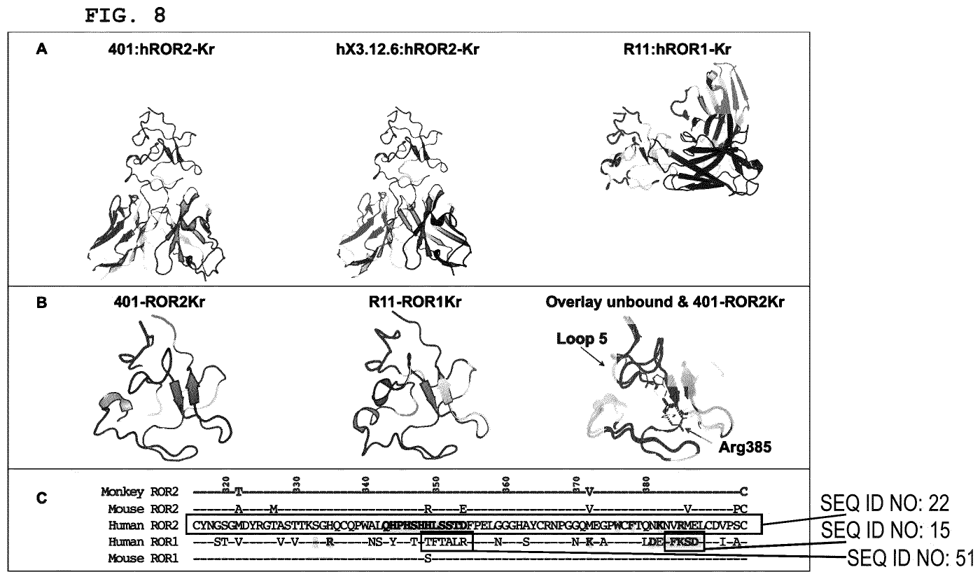

Amino acid sequences:
XBR2-401 VH sequence (SEQ ID No: 1):
QSVKESEGGLFKPTDTLTLTCTVSGFSLSSYGVTWVRQAPGSGLEWIGYINTAGNTYYASWAKSRSTITRN
TNENTVTLKMTSLTAADTATYFCARDWTSLNIWGPGTLVTVSS
XBR2-401 VL sequence (SEQ ID No: 2):
DPMLTQTPSSTSTAVGDTVTIKCQASQSISSDLSWYQQKPGQRPKLLIYQASTLASGVPSRFKGSGYGTEY
TLTISGVQREDAAIYYCLGGYADASYRTAFGGGTKLEIK
X3.12 VH sequence (SEQ ID NO: 3):
QSVKESEGGLFKPTDTLTLTCTVSGFSLSSYGVTWVRQAPGSGLEWIGYINTAGNTYYASWAKSRSTITRN
TNENTVTLKMTSLTAADTATYFCARDDRWSLNIWGPGTLVTVSS
X3.12 VL sequence (SEQ ID NO: 4):
DPMLTQTPSSTSTAVGDTVTIKCQASQSISSDLSWYQQKPGQRPKLLIYQASTLASGVPSRFKGSGYGTEY
TLTISGVQREDAAIYYCLGGYADASYRTAFGGGTKLEIK
hX3.12.5 VH sequence (SEQ ID NO: 5):
EVQLVESGGGLIQPGGSLRLSCAASGFTVSSYGVTWVRQAPGKGLEWVSYINTAGNTYYASWAKSRFTIS
RDNSKNTLYLQMNSLRAEDTAVYYCARDDRWSLNIWGQGTLVTVSS
hX3.12.5 VL sequence (SEQ ID NO: 6):
DPMLTQSPSSLSASVGDRVTITCQASQSISSDLSWYQQKPGKAPKLLIYQASTLASGVPSRFKGSGYGTEY
TLTISSLQPEDFATYYCLGGYADASYRTAFGGGTKLEIK
hX3.12.6 VH sequence (SEQ ID NO: 7):
EVQLVESGGGLVQPGGSLRLSCAASGFTFSSYGVTWVRQAPGKGLEWVSYINTAGNTYYASWAKSRFTIS
RDNAKNSLYLQMNSLRAEDTAVYYCARDDRWSLNIWGQGTLVTVSS
4(GS)x3 linker (SEQ ID NO: 8): GGGGSGGGGSGGGGS
hinge, CH2 (aglycosylation mutation) and CH3 (N297A) (SEQ ID NO: 9):
EPKSSDKTHTCPPCPAPELLGGPSVFLFPPKPKDTLMISRTPEVTCWVDVSHEDPEVKFNWYVDGVEVHN
AKTKPREEQYASTYRWVSVLTVLHQDWLNGKEYKCKVSNKALPAPIEKTISKAKGQPREPQVYTLPPCRDE
LTKNQVSLWCLVKGFYPSDIAVEWESNGQPENNYKTTPPVLDSDGSFFLYSKLTVDKSRWQQGNVFSCSV
MHEALHNHYTQKSLSLSPGA
V9 scFV heavy chain variable domain (VH) (SEQ ID NO: 10):
EVQLVESGGGLVQPGGSLRLSCAASGYSFTGYTMNWVRQAPGKGLEWVALINPYKGVSTYNQKFKDRFTI
SVDKSKNTAYLQMNSLRAEDTAVYYCARSGYYGDSDWYFDVWGQGTLVTV
(G4S)x3 linker (SEQ ID NO: 11): SSGGGGSGGGGSGGGGS
V9 scFV light chain variable domain (VL) (SEQ ID NO: 12):
DIQMTQSPSSLSASVGDRVTITCRASQDIRNYLNWYQQKPGKAPKLLIYYTSRLESGVPSRFSGSGSGTDY
TLTISSLQPEDFATYYCQQGNTLPWTFGQGTKVEIK
hinge, CH2 (aglycosylation mutation) and CH3 (N297A) (SEQ ID NO: 13):
EPKSSDKTHTCPPCPAPELLGGPSVFLFPPKPKDTLMISRTPEVTCWVDVSHEDPEVKFNWYVDGVEVHN
AKTKPREEQYASTYRWVSVLTVLHQDWLNGKEYKCKVSNKALPAPIEKTISKAKGQPREPQVCTLPPSRDE
LTKNQVSLSCAVKGFYPSDIAVEWESNGQPENNYKTTPPVLDSDGSFFLVSKLTVDKSRWQQGNVFSCSV
MHEALHNHYTQKSLSLSPGA
GMCSF signal peptide (SEQ ID NO: 14): MLLLVTSLLLCELPHPAFLLIP
Partial amino acid sequence of human ROR1 from Figure 8 (SEQ ID NO: 15): FKSD
Further partial amino acid sequence of human ROR1 from Figure 8 (SEQ ID NO: 51): TFTALR
IgG4 hinge (SEQ ID NO: 16): ESKYGPPCPPCP
CD28 transmembrane domain (SEQ ID NO: 17): MFWVLVWVGGVLACYSLLVTVAFIIFWV
CD3z signaling domain (SEQ ID NO: 18):
RVKFSRSADAPAYQQGQNQLYNELNLGRREEYDVLDKRRGRDPEMGGKPRRKNPQEGLYNELQKDKMA
EAYSEIGMKGERRRGKGHDGLYQGLSTATKDTYDALHMQALPPR
T2A ribosomal skipping sequence (SEQ ID NO: 19): LEGGGEGRGSLLTCGDVEENPGPR
EGFRt(SEQ ID NO: 20):
RKVCNGIGIGEFKDSLSINATNIKHFKNCTSISGDLHILPVAFRGDSFTHTPPLDPQELDILKTVKEITGFLLIQA
WPENRTDLHAFENLEIIRGRTKQHGQFSLAWVSLNITSLGLRSLKEISDGDVIISGNKNLCYANTINWKKLFGT
SGQKTKIISNRGENSCKATGQVCHALCSPEGCWGPEPRDCVSCRNVSRGRECVDKCNLLEGEPREFVEN
SECIQCHPECLPQAMNITCTGRGPDNCIQCAHYIDGPHCVKTCPAGVMGENNTLVWKYADAGHVCHLCHP
NCTYGCTGPGLEGCPTNGPKIPSIATGMVGALLLLLVVALGIGLFM
Human 1g heavy chain signal peptide (SEQ ID NO: 21): MDWTWRILFLVAAATGAHS
Partial amino acid sequence of human ROR2 (SEQ ID NO: 22):

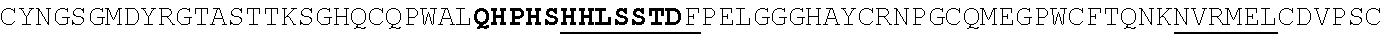

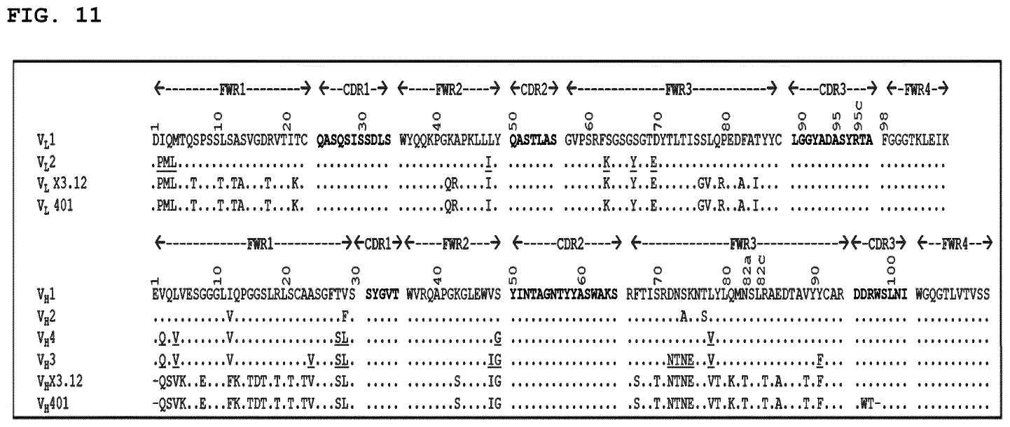

HCDR3 sequence from Figure 9 (SEQ ID NO: 23): DXXSLNI
(note that X is an amino acid which can be selected from any naturally occurring amino acid)
HCDR3 sequence from Figure 9 (SEQ ID NO: 24): DXXXSLNI
(note that X is an amino acid which can be selected from any naturally occurring amino acid)
HCDR3 sequence from Figure 9 (SEQ ID NO: 25): DXXXXSLNI
(note that X is an amino acid which can be selected from any naturally occurring amino acid)
HCDR3 sequence from Figure 10 (SEQ ID NO: 26): DDRWSLNI
HCDR3 sequence from Figure 10 (SEQ ID NO: 27): DKGWSLNI
HCDR3 sequence from Figure 10 (SEQ ID NO: 28): DTMSLNI
HCDR3 sequence from Figure 10 (SEQ ID NO: 29): DWGNWSLNI
HCDR3 sequence from Figure 10 (SEQ ID NO: 30): DYTSLNI
HCDR3 sequence from Figure 10 (SEQ ID NO: 31): DSMSLNI
HCDR3 sequence from Figure 10 (SEQ ID NO: 32): DGLTSLNI
HCDR3 sequence from Figure 10 (SEQ ID NO: 33): DYMMNSLNI
HCDR3 sequence from Figure 10 (SEQ ID NO: 34): DSRNSLNI
HCDR3 sequence from Figure 10 (SEQ ID NO: 35): DSGVVSLNI
HCDR3 sequence from Figure 10 (SEQ ID NO: 36): DWTSLNI
HCDR3 sequence from Figure 10 (SEQ ID NO: 37): DNGSTSLNI
HCDR3 sequence from Figure 10 (SEQ ID NO: 38): DSRRKSLNI
VL1 sequence from Figure 11 (SEQ ID NO: 39):
DIQMTQSPSSLSASVGDRVTITC QASQSISSDLS WYQQKPGKAPKLLLY QASTLAS
GVPSRFSGSGSGTDYTLTISSLQPEDFATYYC LGGYADASYRTA FGGGTKLEIK
VH1 sequence from Figure 11 (SEQ ID NO: 40):
EVQLVESGGGLIQPGGSLRLSCAASGFTVS SYGVT WVRQAPGKGLEWVS YINTAGNTYYASWAKS
RFTISRDNSKNTLYLQMNSLRAEDTAVYYCAR DDRWSLNI WGQGTLVTVSS
LCDR1 sequence from Figure 11 (SEQ ID NO: 41):
QASQSISSDLS
LCDR2 sequence from Figure 11 (SEQ ID NO: 42):
QASTLAS
LCDR3 sequence from Figure 11 (SEQ ID NO: 43):
LGGYADASYRTA
HCDR1 sequence from Figure 11 (SEQ ID NO: 44):
SYGVT
HCDR2 sequence from Figure 11 (SEQ ID NO: 45):
YINTAGNTYYASWAKS
HCDR3 sequence from Figure 11 (SEQ ID NO: 52):
DDRWSLNI
Partial amino acid sequence of VhX3.12 and Vh401 from Figure 11 (SEQ ID NO: 53):
QSVK
Partial amino acid sequence of Vh3, VhX3.12 and Vh401 from Figure 11 (SEQ ID NO: 54):
NTNE
Nucleic acid sequences:
STOP primer (SEQ ID NO: 46): ACCTATTTCTGTGCGAGGATTGTAATCCCTTAACATCTGGGACCA
Unirev primer (SEQ ID NO: 47): ATCTCTCGCACAGAAATAGGT
X2 primer (SEQ ID NO: 48): ACCTATTTCTGTGCGAGAGATNNKNNKTCCCTTAACATCTGGGGACCA
X3 primer (SEQ ID NO: 49):
ACCTATTTCTGTGCGAGAGATNNKNNKNNKTCCCTTAACATCTGGGGACCA
X4 primer (SEQ ID NO: 50):
ACCTATTTCTGTGCGAGAGATNNKNNKNNKNNKTCCCTTAACATCTGGGGACCA

### EXAMPLES

The present invention is exemplified by the following non-limiting examples:

### Example 1: Affinity Maturation, Humanization, and Co-Crystallization of a Rabbit Anti-Human ROR2 Monoclonal Antibody for Therapeutic Utility

Materials and Methods:

Cell Lines and Primary Cells

Breast cancer cell lines MDA-MB-231 and T47D and were purchased from ATCC and grown in DMEM (Thermo Fisher Scientific), 100 U/mL penicillin/streptomycin (Thermo Fisher Scientific), and 10% (v/v) FBS (BioFluid Technologies). Renal cell adenocarcinoma cell line 786-O (an NCI-60 panel cell line obtained from The Scripps Research Institute's Cell-Based High-Throughput Screening Core) were grown in RPMI 1640 (Thermo Fisher Scientific), 100 U/mL penicillin/streptomycin (Thermo Fisher Scientific), and 10% (v/v) FBS (BioFluid Technologies). HEK 293F cells stably transfected with human ROR2 (allotype Thr245), mouse ROR2, and mock vector were previously published [29]. The same study showed that mAb 401 binds to both ROR2 allotypes, Thr245 and Ala245, which arise from a single-nucleotide polymorphism (SNP; rs10820900) in the frizzled domain of human ROR2 (hROR2-Fz). Human PBMCs were purchased from AllCells and cultured in X-VIVO 20 medium (Lonza) with 5% (v/v) off-the-clot human AB serum (Gemini Bio-Products) and 100 U/mL IL-2 (Cell Sciences). Primary T cells were expanded from PBMCs as previously described [34] by using Dynabeads ClinExVivo CD3/CD28 (Thermo Fisher Scientific). The Jurkat-T Lucia NFAT reporter cell line was purchased from InvivoGen and cultured in RPMI 1640 (ATCC modification) medium with 100 U/mL penicillin/streptomycin and 10% (v/v) FBS (BioFluid Tech.); 100 μg/mL zeocin (InvivoGen) was added for every other passage.

Crystallization and structure determination of 401 and hX3.12.6 in complex with hROR2-Kr Cloning, expression, and purification—DNA fragments encoding Fab 401, Fab hX3.12.6 and full-length human ROR2 (clone ID 40146553; GE Healthcare Dharmacon) were each amplified by PCR to create the corresponding scFv formats (V_{H}-3×(GGGGS)-V_{L}) and the kringle domain of human ROR2 (hROR2-Kr). The PCR product encoding hROR2-Kr was cloned into a pET15b expression vector (Novagen), which was modified to co-express E. coli chaperone/disulfide-isomerase (DsbC) [35], to create pET15b-hROR2-Kr-DsbC which expresses hROR2-Kr with a thrombin cleavable N-terminal hexa-histidine tag. The scFv-encoding PCR products containing their own ribosome binding sites were inserted between hROR2-Kr and DsbC affording pET15b-hROR2-Kr-scFv401-DsbC and pET15b-hROR2-Kr-scFv hX3.12.6-DsbC. The 3 expression plasmids were each transformed into E. coli bacteria of the Rosetta-gami2(DE3) strain (Novagen). Transformed E. coli were grown in LB medium containing ampicillin, tetracyclin, and chloramphenicol, at 37° C., with 230 rpm agitation. Protein expression was induced with 0.3 mM isopropyl β-D-thiogalactoside (IPTG) when the cell density reached OD₅₉₅ of 0.6. The cells were grown further for 18 h at 20° C.

Protein Purification—Bacterial pellets were resuspended in sonication buffer (20 mM HEPES, pH 8.0, 500 mM NaCl, 15 mM imidazole, 10% (v/v) glycerol), sonicated in an ice-water bath, and centrifuged for 25 min at 53,300×g. The supernatants were loaded on a custom-packed 10-mL HIS-Select column (Sigma-Aldrich) and washed with sonication buffer. Bound proteins were eluted with a linear gradient of imidazole from 15 mM to 500 mM. The eluted proteins were treated overnight at 4° C. with thrombin (Sigma-Aldrich) to remove the N-terminal hexa-histidine tags at hROR2-Kr. The cleaved proteins were purified further on a Superdex 200 26/60 column (GE Healthcare) equilibrated with 50 mM NaCl, 10 mM HEPES, pH 7.4.

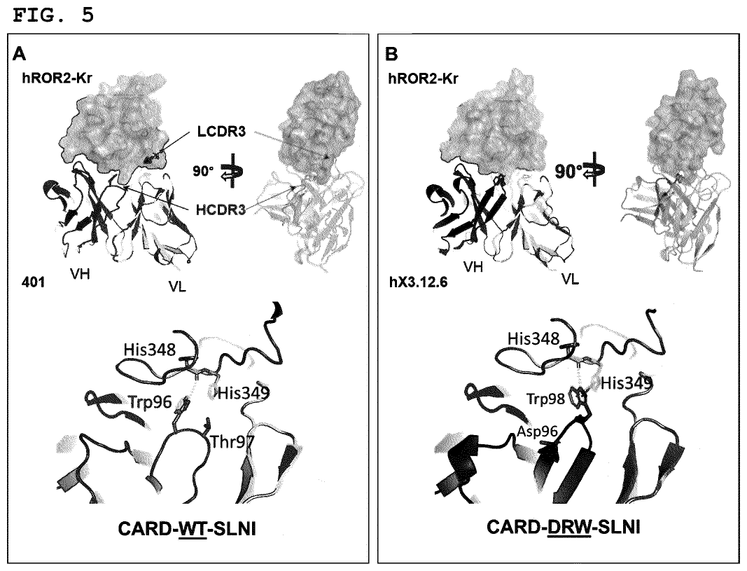

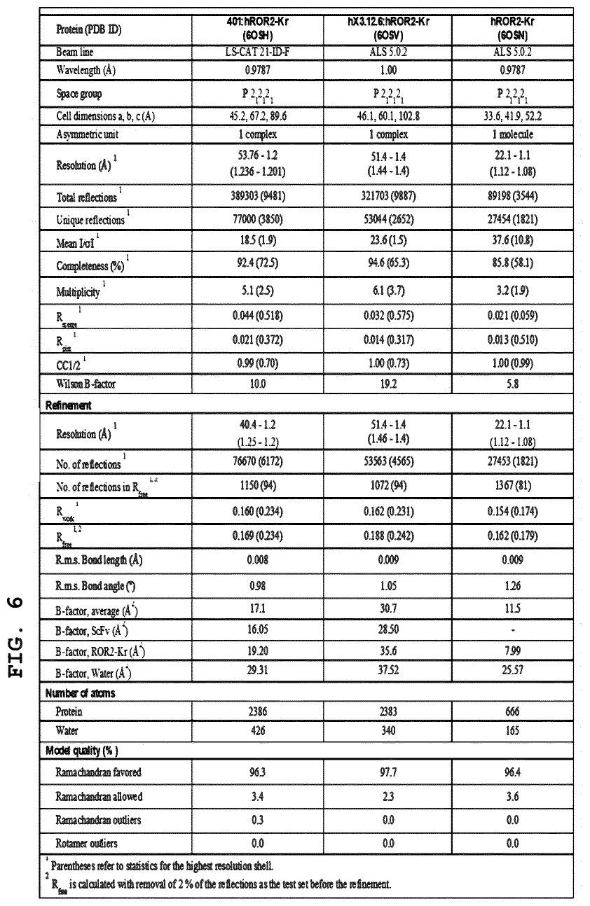

Crystallization and structure determination—Crystals of the scFv401:hROR2-Kr complex were grown by vapordiffusion at room temperature (RT) using 1.5 μL of 14 mg/mL protein and an equal volume of precipitant containing 0.1 M sodium citrate tribasic dihydrate, 15% (w/v) polyethylene glycol 3350, and were fully grown within 2 d. hROR2-Kr produced clustered crystals in vapor diffusion at RT using 2 μL of 14 mg/mL protein with 1 μL of precipitant containing 0.2 M lithium acetate, 20% (w/v) PEG 3350. The crystal clusters were crushed and seeded to drops equilibrated with the protein:precipitant ratio of 3:1 to obtain single crystals. Crystals of the scFv hX3.12.6:hROR2-Kr complex were grown by vapor diffusion at RT using 1.5 μL of 3 mg/mL protein and an equal volume of precipitant containing 10 mM MgCl₂ hexahydrate, 5 mM nickel (II) chloride hexahydrate, 0.1 M Na-HEPES pH 7.0, 13% (w/v) PEG 4000. The crystals were flash frozen in liquid nitrogen using nylon loops after removing excess mother liquor. Diffraction data sets with Bragg spacings set to 1.1 Å for both scFv401:hROR2-Kr and hROR2-Kr were collected on a Rayonix MX300 detector at the Advanced Photon Source (APS) beamline LS-CAT 21-ID-F synchrotron facility (Argonne National Laboratory). A diffraction data set with Bragg spacings set to 1.3 Å for scFv hX3.12.6:hROR2-Kr was collected on a PILATUS3 S 6M detector at the Advanced Light Source (ALS) beamline 5.0.2 synchrotron facility (Lawrence Berkeley National Laboratory). Datasets were processed with autoPROC using XDS as the processing engine [36]. The structures were solved by the molecular replacement method using PHASER with PDB ID 6BA5 (scFvR11:hROR1-Kr) [34] as the search model. Crystallographic refinements were performed using PHENIX version 1.14 [38]. Manual rebuilding and adjustment of the structures were done in Coot [39]. Data processing and refinement statistics are shown in (FIG. 6). Molecular images (FIG. 5A,B and FIG. 8A,B) were created using PyMOL [40]. Interaction interfaces were analyzed using PDBePISA [41, 42]. Structure validations were carried out with MolProbity [43].

Affinity Maturation

Library generation—A stop mutant of 401 was generated using overlap extension PCR with sense primer STOP and antisense primer unirev to avoid contamination of the library with parental 401. Subsequently, 3 different libraries were generated to randomize and extend HCDR3 with NNK codons; 2 randomized codons (X2) located at residues 96 and 97 on HCDR3, an additional randomized codon immediately downstream of residue 97 (×3), and 2 additional randomized codons immediately downstream of residue 97 (×4). These variants were generated by overlap extension PCR utilizing the NNK-degenerated sense primers X2, X3 or X4 together with antisense primer unirev. The final constructs were cloned into phagemid pC3C as previously described along with flanking primers C-5′SF IVL & c-3′sfivh [29] Primer sequences: STOP, 5′-ACCTATTTCTGTGCGAGGATTGTAATCCCTTAACATCTGGGGACCA-3′; unirev, 5′-ATCTCTCGCACAGAAATAGGT-3′; X2 5′-ACCTATTTCTGTGCGAGAGATNNKNNKTCCCTTAACATCTGG GGACCA-3′; X3 5′-ACCTATTTCTGTGCGAGAGATNNKNNKNNKTCCCTTAACATCTGGGGACCA-3′; X4 5′-ACCTATTTCTGTGCGAGAGATNNKNNKNNKNNKTCCCTTAACATCTGGGGACCA-3′.

Library selection—Following published protocols for the selection of chimeric rabbit/human Fab by phage display [44], 3 different panning approaches were investigated where the first was conventional surface panning (3 rounds) using 1 μg of the hROR2-Fc [29] in 25 μL PBS for immobilization on a 96-well ELISA plate (Costar 3690; Corning), 3% (w/v) BSA in PBS for blocking, and 10 wash steps using 0.05% (v/v) Tween 20 in PBS (TPBS). The second approach was surface competition panning (using the second round of the conventional surface panning, one round was conducted) using 100 ng hROR2-Fc, immobilized and blocked as above, followed by a 2-h pre-incubation with a 10-fold molar excess of the parental Fab 401 and 15 wash steps with TPBS. The third approach was solution competition panning (5 rounds) using 2-fold decreasing amounts of biotinylated [44] hROR2-Fc (100 to 6.25 ng in PBS), pre-incubation with a 10-fold molar excess of the parental Fab 401 to the hROR2-Fc at the various concentrations. At each step, capturing with streptavidin-coated magnetic beads (Dynabeads MyOne Streptavidin C1; Thermo Fisher Scientific), acid elution using 100 mM glycine-HCl (pH 2.2), coupling of panning rounds 2, 3 and 4, 5 (i.e., without intermittent reamplification) and increasingly stringent wash steps (0.05% (v/v) to 0.5% (v/v) Tween 20 in PBS) was conducted. Using published protocols [44], final output colonies were screened by a Fab ELISA using immobilized hROR2-Fc, and the HCDR3 of positive clones was determined by DNA sequencing.

Humanization Humanization of X3.12 was done by finding the closest human germline(s) using IgBlast (www.ncbi.nlm.nih.gov/igblast/) with the least amount of polymorphisms, which was determined using IMGT's IGHV and IGKV mammalia human (Homo sapiens) links (www.imgt.org/IMGTrepertoire/Proteins/). Rational mutations [45] were performed in varying severity to determine mutations that were necessary to retain ROR2 affinity.

Fab Cloning, Expression, and Purification

All selected Fab variants were cloned as described with modifications [29]. Briefly, Fab variants were cloned into bacterial expression vector pET11a [46], transformed into E. coli strain Rosetta (DE3) (EMD Millipore), and tandemly purified from culture supernatants using a 1-mL HiTrap Kappa Select HP column followed by a 1-mL HisTrap HP column in conjunction with an AKTA FPLC instrument (all from GE Healthcare). (The initial top 12 chimeric rabbit/human anti-human Fabs were purified using only the 1-mL HisTrap HP column). Purity of protein was analyzed by SDS-PAGE, Coomassie Blue staining, and A280 absorbance was used to determine the concentration of purified Fab variants.

Surface Plasmon Resonance

Kinetic and thermodynamic parameters for the ROR2 binding of purified anti-ROR2 Fab variants were measured by use of SPR as previously described [29], performed on a Biacore X100 instrument using Biacore reagents and software (GE Healthcare). Briefly, a CM5 sensor chip was immobilized with a mouse anti-human IgG C_{H}2 mAb in order to capture the hROR2-Fc antigen. Each Fab variant was diluted to 100 nM 1×HBS-EP+ running buffer and further diluted 2-fold using 1×HBS-EP+ running buffer to make 5 dilutions total with a replicate of the lowest concentration after measuring the highest concentration to confirm regeneration of the sensor chip.

Thermostability Assay

Steps were followed as described in the LightCycler 480 Instrument Quick Guide (Roche) for protein melting. Optimal conditions were determined using parental Fab 401 at 1 mg/mL and the suggested Optimization Table 1 in the Quick Guide. Roche Protein Melting software was used for analysis. The optimal conditions required 0.5 μL 1 mg/mL Fab, 1.0 μL SYPRO Orange Dye 100×stock, 8.50 μL Dulbecco's PBS (DPBS). All Fabs were tested under these conditions and in triplicates.

Retrogenix Cell Microarray

Custom pre-screens, full screens, and post-screens were carried out by Retrogenix as described previously [29, 47].

Functionality Studies

Production of ROR2×CD3 bispecific antibody—Cloning, expression, and purification of ROR2×CD3 biAbs in heterodimeric aglycosylated scFv-Fc format followed a previously described protocol with modifications [48]. In short, the scFv encoding sequences were synthesized as gBlocks containing a signal peptide encoding sequence at the N-terminus (Integrated DNA Technologies). Overlap extension PCR was used to include sequences encoding hinge and heavy chain constant domains C_{H}2 and C_{H}3 of human IgG1. Previously described hole and knob mutations [49] were included in the CD3 scFv-hinge-C_{H}2-C_{H}3 and ROR2 scFv-hinge-C_{H}2-C_{H}3 encoding sequences, respectively. The aglycosylation mutation N297A in C_{H}2 was included in both. These scFv-Fc encoding sequences were then inserted into mammalian expression vector pCEP4 using KpnI and XhoI restriction sites. Following DNA sequencing (Eton Bioscience) for verification, the plasmids were transfected into HEK 293F cells (Thermo Fisher Scientific) using polyethylenimine (PEI; Polysciences) at 3×10⁶ cells/mL cultured in 150 mL FreeStyle medium (Thermo Fisher Scientific) shaking at 37° C. in an atmosphere of 8% CO₂ and 100% humidity. After 6-12 h, an additional 150 mL FreeStyle media was added. Supernatants were collected after 3 d followed by filtration and purification using a 1-mL HiTrap Protein A HP column (GE Healthcare) in conjunction with an AKTA FPLC instrument (GE Healthcare) followed by size exclusion chromatography using a Superdex 200 10/300 GL column (GE Healthcare Life Sciences) equilibrated with water followed by DPBS. Yields were typically ˜5-10 mg/L. The purity of the biAbs was confirmed by SDS-PAGE followed by Coomassie blue staining and quantified by A280 absorbance.

Flow cytometry—Similar to standard methods and as previously described [29, 34] staining of 1×10⁵ target cells was done with 5 μg/mL Fab or biAb in 100 μL cytometry buffer (PBS supplemented with 1% (w/v) BSA, and 0.1% (w/v) sodium azide). After washing, the cells were incubated with a 1:1,000 dilution of PE-conjugated goat anti-human IgG F(ab′)₂ fragment specific pAbs or Alexa Fluor 647-conjugated donkey anti-goat IgG (H+L) pAbs (both in F(ab′)₂ format from Jackson ImmunoResearch) in 100 μL flow cytometry buffer on ice for 1 h. Alexa Fluor 647-conjugated mouse anti-human CD69 mAb was purchased from BioLegend. Cells were analyzed using a FACSCanto instrument (BD Biosciences) and FlowJo analytical software (Tree Star).

In vitro cytotoxicity and T-cell activation assays—Cytotoxicity was measured by using CytoTox-Glo (Promega) following the manufacturer's protocol and a previous publication [48] with minor modifications. Primary T cells expanded from healthy donor PBMCs were used as effector cells and 786-O or MDA-MB-231 cells were used as target cells at an effector-to-target cell ratio of 10:1. Cells were incubated in X-VIVO 20 medium (Lonza) with 5% (v/v) off-the-clot human AB serum. Target cells (2×10⁴) were first incubated with the biAbs before adding the effector cells (2×10⁵) in a final volume of 100 μL per well in a 96-well tissue culture plate followed by incubation at 37° C. for 16 h. A biAb concentrations range from 2 ng/mL to 1 μg/mL was used. Plates were centrifuged and 50 μL of the supernatants were transferred into a 96-well clear-bottom white-walled plate (Costar 3610; Corning) containing 25 μL per well CytoTox-Glo reagent. After 15 min incubation at RT, a SpectraMax M5 instrument was used to read the plates with SoftMax Pro software set to luminescence. Following the ELISA Ready-SET-Go! Reagent protocols (eBioscience), additional supernatant from the previous study were diluted 20-fold and used a human IFN-γ ELISA.

Results:

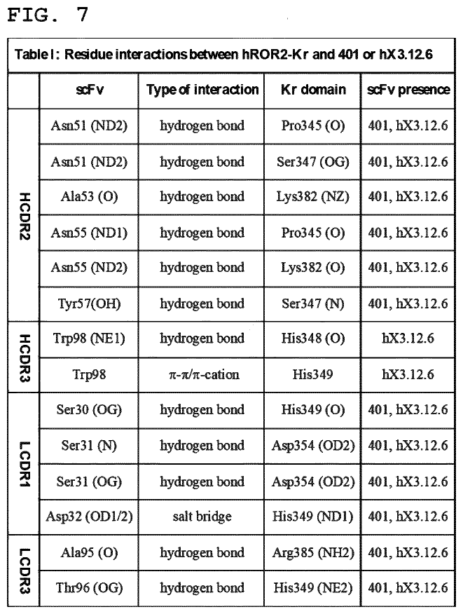

Crystallization of mAb XBR2-401 in complex with the human ROR2 kringle domain We previously reported a panel of 12 chimeric rabbit/human Fabs that were selected from a naïve rabbit antibody library for binding to human ROR2 [29]. Among these, mAb XBR2-401 (“401”) in Fab and IgG1 format (FIG. 19) was shown to be specific for the kringle domain of ROR2 (hROR2-Kr) and to recognize both human and mouse orthologs but not its closest relative, ROR1 [29]. To define the 401 paratope and epitope, we used X-ray crystallography to solve the structure of 401 in scFv (FIG. 19) format in complex with hROR2-Kr at 1.2-Å resolution (FIG. 6) (Protein Data Bank ID (PDB): 60SH) (FIG. 5A). The crystal contained one complex in the asymmetric unit. All residues in the crystal were well resolved except the 15-amino acid scFv linker. The buried surface area between 401 and hROR2-Kr was 720 Å², which comprised 7.0% and 15.9% of the total surface area of 401 and hROR2-Kr, respectively. The van der Waals contacts were dominated by HCDR2 and LCDR3 of 401. Notably, Ala95 (Kabat numbering) from LCDR3 was nestled in a shallow hydrophobic pocket created by hROR2-Kr loop 3, 5, and 6 residues [50], Leu350, Pro368, GIn371, Trp376, Phe378, and Met386. On the other hand, His349 of loop 3 from hROR2-Kr projected into the main pocket formed by the CDRs and made a salt bridge to Asp32 of LCDR1 (FIG. 7). The interface also contained numerous direct and water-mediated hydrogen bond interactions dominated by residues from HCDR2 and LCDR1 (FIG. 7). Compared to the residues from HCDR2 and LCDR3 that are heavily involved in epitope recognition, Trp96 (Kabat numbering) from HCDR3 provided limited interactions with hROR2-Kr through a suboptimal hydrogen bond with His348 and van der Waals interactions with His349 with the potential to form a π-π bond (FIG. 5A). This observation posed an opportunity for optimizing HCDR3 binding to the kringle domain.

hROR2-Kr and hROR1-Kr share 58% amino acid sequence identity [11]. When the epitope residues of hROR2-Kr that are recognized by 401 were compared to those mediating R11:hROR1-Kr recognition [34], no residue overlap was found (FIG. 8). This observation explains why there is no cross-reactivity between 401 and R11 despite hROR2-Kr and hROR1-Kr's homologous amino acid sequences. When comparing the antibody bound kringle domains from the 401:hROR2-Kr and the R11:hROR1-Kr complex, the root-mean-square deviation (rmsd) of Ca positions was found to be 0.695 Å, revealing a highly conserved tertiary structure of the two Kr domains.

The inventors also crystallized and solved the structure of antibody unbound hROR2-Kr at 1.1-Å resolution (FIG. 8B right). The rmsd of the unbound hROR2-Kr structure to 401-bound hROR2-Kr was 0.383 Å revealing only minor differences between the coordinates. Notably, in the unbound hROR2-Kr structure, Arg385 formed a mixed salt bridge/hydrogen bond interaction with an acetate from the crystallization solution (FIG. 8B right). The binding site, which overlaps with the canonical lysine binding sites (LBS) in other kringle domains [51], was partially covered by 401 in the crystal structure of the 401:hROR2-Kr complex. Superposition of the unbound hROR2-Kr structure to the 401-bound hROR2-Kr showed a minor shift in loop 5 due to the bound acetate ion (FIG. 8B right).

Overall, the inventors' findings of no overlap between ROR2 and ROR1 epitopes along with suboptimal binding of 401's HCDR3 to hROR2-Kr revealed opportunities for in vitro affinity maturation.

Affinity Maturation Via Phage Display

A phage display library was constructed to conduct focused mutagenesis on 401's HCDR3 residues 96 and 97 (Kabat numbering; FIG. 11) with 0, 1, or 2 additional randomized residues (FIG. 9). Additional randomized positions were investigated because the co-crystal structure of 401:hROR2-Kr depicted an open cavity between hROR2 and the HCDR3 which could be filled by a longer matured HCDR3 and improve mAb affinity. Selection for hROR2-Fc binding was performed 3 ways: surface, surface competition, and solution competition panning (see Experimental Procedures). Both competition panning protocols applied selection pressure towards Fabs with lower dissociation rate constants (k_{off}) and thus higher affinity. From the 3 combined libraries, 144 clones were selected and analyzed via ELISA for ROR2 binding and Fab expression from supernatants. The top 12 clones with the highest absorbance ratio (hROR2 binding to expression) were purified. Using surface plasmon resonance (SPR), thermodynamic (K_{D}) and kinetic parameters (kₒₙ and k_{off}) of the interaction with hROR2 were determined. Clone XBR2-401-X3.12 (“X3.12”), which was obtained from the ×3 library, revealed the highest affinity (K_{D}=0.7 nM) (FIG. 10, FIG. 14A), which is at least a 5-fold improvement from 401. The X3.12 HCDR3 sequence differs from 401 at 2 residue positions, and is one residue longer, changing the HCDR3 sequence from DWTSLNI to DDRWSLNI (FIG. 10). To make the affinity matured X3.12 more therapeutically relevant, the next step was to employ humanization.

Humanization by CDR Grafting

Humanization of the affinity-matured chimeric rabbit/human X3.12 Fab was performed in 3 main steps. First, the human germlines with the closest identity to X3.12's variable light chain (V_{L}) and variable heavy chain (V_{H}) amino acid sequences were identified using IgBlast (see Experimental Procedures for online tools). The IMGT repertoire was then referenced to eliminate germlines with >3 polymorphisms. Human germlines which had the highest amino acid sequence identity to X3.12 with the least number of polymorphisms were heavy chain germlines IGHV3-66*03, IGHV3-48*03, and light chain germline IGKV1-NL1*01 which are 54.3%, 54.8%, and 65.6% identical to the X3.12 heavy and light chain, respectively. Second, CDRs from X3.12 determined using Kabat numbering were grafted into these 3 framework sequences (FIG. 11, V_{L}1, V_{H}1, V_{H}2). Third, residues determined to preserve affinity [45] were back mutated from the human germline residues to the original rabbit residues. From these 3 steps comprised of CDR grafting and rational back mutations, 4 heavy chain (V_{H}1-V_{H}4) and 2 light chain variants (VA V_{L}2) were formed (FIG. 11). These humanized chains were compared to the IMGT database of human germline antibody sequences using the IMGT/DomainGapAlign tool to determine the human identity percentage. The human identity of V_{L}1, V_{L}2, and V_{H}1-V_{H}4 was 88.6%*, 87.1%, 86.6%, 87.8%, 73.5%*, and 80.4%*, respectively, where the starred (*) percentages indicate the first “hit” on IMGT DomainGapAlign was not human. WHO standards state the first “hit” on IMGT DomainGapAlign tool must be human along with human identity being above 85% for the antibody to be considered humanized [52]. All heavy and light chain Fab combinations were cloned into a pET11a variant [46] and expressed in the E. coli Rosetta strain followed by quantification of Fab expression and hROR2 binding via ELISA to eliminate nonbinding clones. The remaining clones hX3.12.5, hX3.12.6, hX3.12.7, and hX3.12.8 had higher binding-to-expression ratios compared to X3.12. The overall percent identity of these variants to human germlines was 87%, 87%, 80%, and 84%, respectively, and all utilized V_{L}2 (FIG. 12). Therefore, hX3.12.5 and hX3.12.6 are considered humanized whereas hX3.12.7 and hX3.12.8 are considered chimeric mAbs by WHO standards.

Characterization of Affinity Matured and Humanized Fabs

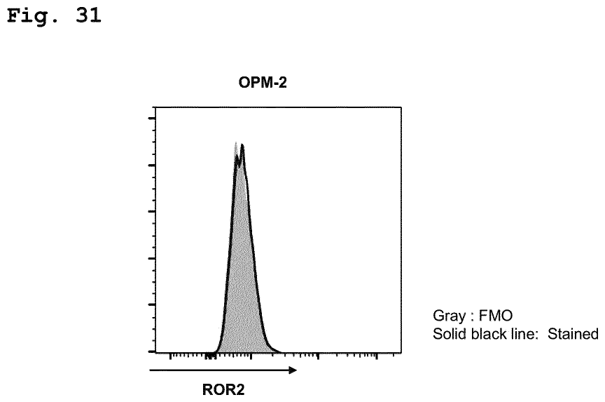

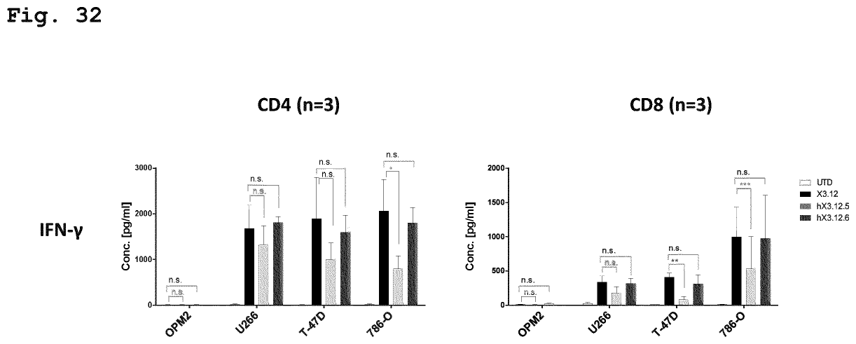

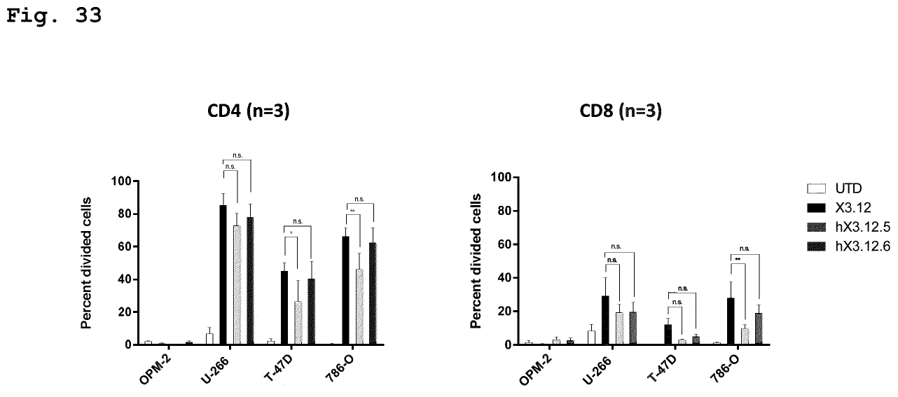

Following expression and purification (FIG. 18A), the affinities of hX3.12.5, hX3.12.6, hX3.12.7, and hX3.12.8 were determined to be 2.6 nM, 3.8 nM, 1.4 nM, and 5.2 nM, respectively, by SPR (FIG. 12, FIG. 14B). Moving forward, we focused on hX3.12.5 and hX3.12.6 Fabs as they have the highest human identity while retaining nanomolar affinity for hROR2. Supporting flow cytometry data showed hX3.12.5 and hX3.12.6 bound HEK 293F cells stably overexpressing hROR2-Thr245 allotype [29], while minimally binding the mock transfected HEK 293F control cell line which has some ROR2 expression (FIG. 13A). A Fab containing hX3.12.6 framework regions and parental 401 CDR sequences was generated and included for reference (h401.6, 16 nM affinity, FIG. 12). Clones hX3.12.5 and hX3.12.6 also bound to breast cancer cell line T47D (ROR2+, ROR1−) and renal cell adenocarcinoma cell line 786-O (ROR2+, ROR1+) but not to breast cancer cell line MDA231 (ROR2−, ROR1+) (FIG. 13A). The thermostability of humanized Fabs was addressed using LightCycler 480 to measure their melting temperatures. Chimeric rabbit/human Fabs X3.12 and 401 exhibited slightly higher thermostability than the humanized Fabs hX3.12.5 and hX3.12.6 along with h401.6 (FIG. 15). The melting temperatures of the affinity matured and humanized Fabs are similar to previously reported Fab melting temperatures and suggest they are stable [53, 54].

To test hX3.12.6's specificity to ROR2, the Fab was first converted to an IgG1-like format, scFv-Fc. This format contains 2 scFv's utilizing a (Gly₄Ser)₃ linker between V_{L} and V_{H} and fused to the human IgG1 Fc fragment (FIG. 19). The hX3.12.6 scFv-Fc was screened against 786-O, T47D, and MDA-MB-231, confirming all to be ROR2+(FIG. 13B) except MDA-MB-231 as previously described. We also confirmed hX3.12.6's cross-reactivity with mouse ROR2 (FIG. 13C) which was stably expressed on HEK 293 as previously described [29]. As we did for the parental mAb XBR2-401 in chimeric rabbit/human IgG1 format [29], the hX3.12.6 scFv-Fc was screened against 5,647 human plasma membrane proteins (i.e., human cell surface antigens) expressed on the surface of human HEK 293 cells and arrayed on 16 slides in duplicate [47]. The spotting pattern of ZsGreen1, which correlates with human cell surface antigen expression (FIG. 16A), is shown for the slide that contained human ROR2 (FIG. 16B). The only specific interaction identified in the hX3.12.6 scFv-Fc screen was ROR2 (FIG. 16B, 16C), confirming that neither affinity maturation nor humanization diminished the high specificity of the parental mAb.

Next, hX3.12.6 scFv was co-crystallized with hROR2-Kr and its structure was determined at 1.4-Å resolution (FIG. 5B, FIG. 6). The structure of the complex of hX3.12.6 and hROR2-Kr allowed us to compare the affinity matured HCDR3 to the parental HCDR3, particularly the π-π interaction formed with hROR2-Kr (FIG. 5B, FIG. 7). Similar to 401, in hX3.12.6 Ala95 was buried in hROR2-Kr and the salt bridge between light chain Asp32 and hROR2-Kr was also retained. All hydrogen bond interactions and van der Waals contacts present in 401:hROR2-Kr remained intact in the hX3.12.6:hROR2-Kr complex except for the changes in the HCDR3 due to affinity maturation which include Asp96 and Arg97. While these 2 residues do not directly interact with hROR2-Kr, they help to properly position Trp98, which does contact hROR2-Kr. Trp98 in hX3.12.6's further improves interactions with hROR2-Kr, having been optimized from 401's Trp96, located at the tip of HCDR3 loop (FIG. 5A, 5B). Unlike in 401, the side chain of Trp98 made an optimal hydrogen bond interaction with the backbone oxygen of hROR2-Kr's His348. The side chain of Trp98 also displayed geometric characteristics of π-π/π-cation interactions with hROR2-Kr His349 (FIG. 5B bottom, FIG. 7). The rmsd between 401 and hX3.12.6 in their respective co-crystal structures was found to be 0.446 Å, suggesting subtle differences between the structures (FIG. 8A). The crystallized kringle domains in complex with either 401 or hX3.12.6 had a rmsd of 0.279 Å, indicating no relevant change between the 2 kringle domains. Collectively, these findings confirmed our rational design of affinity maturation was critical in the improvement of ROR2 binding.

Generation and characterization of ROR2×CD3 bispecific antibodies In order to determine the affinity matured and humanized antibodies' functionality, hX3.12.6 was converted to a ROR2×CD3 biAb and purified by Protein A and SEC (FIG. 19, FIGS. 18B&C). We used the same heterodimeric and aglycosylated scFv-Fc format we previously reported for ROR1×CD3 and CD19×CD3 biAbs [34, 48]. This was done by combining aglycosylation mutation N297A in the C_{H}2 domain with knobs-into-holes mutations in the C_{H}3 domain, specifically C_{H}3 knob mutations S354C and T366W and C_{H}3 hole mutations Y349C, T366S, L368A, and Y407V [49, 55, 56]. As for the anti-CD3 arm, the well-defined affinity matured and humanized anti-human CD3 mAb v9 was used [57]. BiAbs were confirmed to bind 786-O and Jurkat-T Lucia (CD3+) while not binding MDA-MB-231 (FIG. 17A). Primary T-cells were expanded in vitro from 2 different healthy donor peripheral blood mononuclear cells (PBMCs) by the addition of anti-CD3/anti-CD28 beads and IL-2. Next, in vitro biAb-mediated target-dependent cytotoxicity by the expanded primary T-cells was examined. Specific lysis of 786-O cells was seen for the 3 ROR2×CD3 biAbs; 401×v9, hX3.12.6×v9, and hX3.12.5×v9, and positive control biAb ROR1×CD3 biAb XBR1-402×v9 (402×v9) with EC₅₀ values of 0.19, 0.21, 0.15, and 0.25 μg/mL, respectively (1.9 nM, 2.1 nM, 1.5 nM, and 2.5 nM) (FIG. 17B). A monospecific hX3.12.6 scFv-Fc was used as a negative control. To confirm these biAbs are specifically killing via binding ROR2 on 786-O cells and CD3, the inventors tested a ROR2−, ROR1+ cell line, MDA-MB-231, and found all ROR2×CD3 biAbs were inactive up to 1 μg/mL (FIG. 17B). The positive control ROR1×CD3 biAb did show specific cell lysis as expected due to its binding to ROR1 on MDA-MB-231 cells. T-cell activation was quantified by flow cytometry using an anti-CD69 mAb, a known marker of early T-cell activation. Humanized biAbs hX3.12.6×v9, hX3.12.5×v9, and parental 401×v9 incubated with T cells at 0.2 μg/mL upregulated CD69 on over 50% of T cells in the presence of 786-O but not MDA-MB-231 cells (FIG. 17C). The negative control hX3.12.6 scFv-Fc did not reveal upregulation of CD69. The release of type 1 cytokine IFN-γ was assessed by ELISA where all ROR2×CD3 biAbs caused cytokine release in the presence of ROR2+ but not ROR2− target cells (FIG. 17C). As previously shown [34], R11×v9 caused comparable cytokine release in the presence of ROR1+ target cells.

### Example 2: Preparation and Functional Testing of ROR2-Specific CAR-Modified Human CD8+ and CD4+ T Cells with Affinity Matured Targeting Domains

Materials and Methods:

Human Subjects

Blood samples were obtained from healthy donors who provided written informed consent to participate in research protocols approved by the Institutional Review Board of the University of Würzburg (Universitätsklinikum Würzburg, UKW). Peripheral blood mononuclear cells (PBMC) were isolated by centrifugation over Ficoll-Hypaque (Sigma, St. Louis, Mo.).

Cell Lines

The 293T, MDA-MB231, T-47D, 786-O and U266 cell lines were obtained from the American Type Culture Collection. Luciferase expressing lines were derived by lentiviral transduction of the above-mentioned cell lines with the firefly (P. pyralis) luciferase (ffluc)-gene. The cells were cultured in Dulbecco's modified Eagle's medium supplemented with 10% fetal calf serum and 100 U/ml penicillin/streptomycin or RPMI-1640 medium supplemented with 10% fetal calf serum and 100 U/mL penicillin/streptomycin, respectively.

Cell Line Phenotyping

Tumor cell lines were stained with AF647-conjugated hX3.12.5×h38C2 DVD antibody or matched isotype controls. Staining with 7-AAD (BD Biosciences) was performed for live/dead cell discrimination as directed by the manufacturer. Flow analyses were done on a FACS Canto II (BD) and data was analyzed using FlowJo software (Treestar, Ashland, Oreg.).

Immunophenotyping

PBMC and T cell lines were stained with one or more of the following conjugated mAb: CD3, CD4, CD8 and matched isotype controls (BD Biosciences, San Jose, Calif.). Transduced T cell lines were stained with AF647-conjugated anti-EGFR antibody (ImClone Systems Incorporated, Branchburg, N.J.). Staining with 7-AAD (BD Biosciences) was performed for live/dead cell discrimination as directed by the manufacturer. Flow analyses were done on a FACS Canto II (BD) and data was analyzed using FlowJo software (FlowJo LLC, Ashland, Oreg.).

Lentiviral Vector Construction, Preparation of Lentivirus, and Generation of CAR-T Cells

The construction of epHIV7 lentiviral vectors containing ROR2-specific CARs with optimal spacers and a CD28 or 4-1BB costimulatory domain has been described, see reference [58, 59], which is hereby incorporated by reference in its entirety for all purposes. All CAR constructs encoded a truncated epidermal growth factor receptor (EGFRt; also known as tEGFR), see reference [60], which is hereby incorporated by reference in its entirety for all purposes, downstream of the CAR. The genes were linked by a T2A ribosomal skip element.

CAR/EGFRt and ffluc/eGFP-encoding lentivirus supernatants were produced in 293T cells co-transfected with each of the lentiviral vector plasmids and the packaging vectors pCHGP-2, pCMV-Rev2 and pCMV-G using Calphos transfection reagent (Clontech, Mountain View, Calif.). Medium was changed 16 h after transfection, and lentivirus collected after 72 h. CAR-T cells were generated as described [61]. In brief, CD8+ or CD4+ bulk T cells were sorted from PBMC of healthy donors, activated with anti-CD3/CD28 beads (Life Technologies), and transduced with lentiviral supernatant. Lentiviral transduction was performed on day 2 by spinoculation, and T cells propagated in RPMI-1640 with 10% human serum, GlutaMAX (Life technologies), 100 U/mL penicillin-streptomycin and 50 U/mL IL-2. Trypan blue staining was performed to quantify viable T cells. After expansion, EGFRt+ T cells were enriched by magnetic activated cell sorting and expanded by polyclonal stimulation with the CD3-specific Okt3 antibody and irradiated allogeneic PBMC and EBV-LCL feeder cells.

Cytotoxicity, Cytokine Secretion, and CFSE Proliferation Assays

Target cells stably expressing firefly luciferase were incubated in triplicate at 5×10³ cells/well with effector T cells at various effector to target (E:T) ratios. The decrease in luminescence signal in wells that contained target cells and T cells was measured using a luminometer (Tecan). Specific lysis was calculated using the standard formula using untransduced T cells at the respective E:T to offset TCR mediated cytolysis.

For the analysis of cytokine secretion, 5×10⁴ T cells were plated in triplicate wells with target cells at a ratio of 4:1 and IFN-γ, and IL-2 measured by ELISA (Biolegend) in supernatant removed after a 24-hour incubation.

For proliferation analysis, 5×10⁴ T cells were labeled with 0.2 μM carboxyfluorescein succinimidyl ester (CFSE, Invitrogen), washed and plated in triplicate wells with lethally irradiated target cells at a ratio of 4:1 in CTL medium without exogenous cytokines. After 72 h of incubation, cells were labeled with anti-CD3 or anti-CD4 or anti-CD8 mAb and 7-AAD to exclude dead cells from analysis. Samples were analyzed by flow cytometry and cell division of live T cells assessed by CFSE dilution.

Results

Generation, Detection and Enrichment of ROR2 CAR-T Cells

PBMCs from healthy donors were isolated by Ficoll-Hypaque density gradient centrifugation and bulk CD4+ or CD8+ human T cells were extracted from this cell population using MACS. Directly after isolation the T cells were activated with CD3/28 Dynabeads for two days and then transduced by spinoculation with lentiviral vectors encoding the X3.12-based ROR2-specific CAR at a multiplicity of infection (MOI) of 3. The Dynabeads were removed 4 days after transduction and at day 10 the T cells were enriched for EGFRt-positive cells by labeling with a biotinylated monoclonal αEGFR antibody and MACS with anti-biotin microbeads. After the enrichment, the EGFRt-positive fraction reproducibly accounted for over 90% of total cells (FIGS. 21A and 21B).

Cytolytic Activity of ROR2 CAR-T Cells

X3.12-based ROR2-specific CAR-T cells were generated as described above and their cytolytic activity was assessed in 24 h cytotoxicity assays against the ROR2-positive and ffluc-expressing target cell lines T-47D, 786-O and U266 (see FIG. 20). No specific lysis was detected against ROR2-negative MDA-MB-231 controls. The assay was repeated under the same conditions for n=3 independent healthy donor (FIG. 22).

Effector cytokine secretion following ROR2-specific activation of ROR2 CAR-T cells CD4+ or CD8+ X3.12-based ROR2-specific CAR-T cells were generated as described above and co-cultured with ROR2-expressing target cell lines at an E:T ratio of 4:1 for 24 h. After the incubation the cell culture supernatant was collected and analyzed for the presence of the effector cytokines IL-2 and IFN-γ by ELISA. As controls, the cells were co-cultured with ROR2-negative MDA-MB231 cells or in absence of any target cell (media control). For controlling the general ability of the CAR-T cells to produce the effector cytokines of interest the cells were polyclonally stimulated with a combination of the protein kinase C (PKC)/NF-κB-activator phorbol 12-myristate 13-acetate (PMA) and the Ca2+ ionophore ionomycin. The assay procedure was repeated for n=3 unrelated healthy donors and the measured cytokine concentrations were used for group analysis (FIG. 23).

X3.12-based ROR-specific CART cells showed antigen-dependent cytokine secretion profiles. Additionally, the cytokine secretion level correlated with the ROR2 expression levels on the target cells, with 786-O and U266 showing the highest cytokine secretion levels. In general, CD4+ T cells secreted higher amounts of IL-2 and IFN-γ than CD8+ T cells. IFN-γ was detected exclusively in samples that included ROR2-positive targets or PMA/Iono and the average concentrations were in the range of 400-1000 pg/mL (CD4+ T cells) and 200 to 500 pg/mL (CD8+ T cells). IL-2 was also exclusively detected in samples that included ROR2-positive target cells and the average concentrations were in the range of 300-1000 pg/ml (CD4+ T cells) and 150-300 μg/mL (CD8+ T cells).

Proliferation of ROR2 CAR-T Cells

CD4+ or CD8+ X3.12.-based ROR2-specific CAR-T cells were generated as described, labeled with CFSE and co-cultured with lethally irradiated ROR2-expressing target cell lines at an E:T ratio of 4:1 for 72 h in the absence of exogenous cytokines. After the incubation time the T cells were collected and analyzed for CFSE dilution by flow cytometry. As a negative control, the CAR-T cells were co-cultured with ROR2-negative MDA-MB-231 cells or medium and as a positive control in the presence of 50 UI/ml IL-2.

ROR2-negative MDA-MB-231 and medium alone caused no T cell proliferation. However, high antigen-dependent CAR-T cell proliferation was observed for X3.12-based ROR2-specific CAR T cells against ROR2-positive target cells (FIG. 24). These findings demonstrate that the detected proliferation of ROR2 CAR-T cells was mediated by the CAR, in response to stimulation by ROR2-positive cells.

### Example 3: In-Depth Analysis of Functional Differences Between XBR2.401 and X3.12-Derived ROR2-Specific CAR Knock-In T Cells

Materials and Methods:

Human Subjects

Blood samples were obtained from healthy donors who provided written informed consent to participate in research protocols approved by the Institutional Review Board of the University of Würzburg (Universitätsklinikum Würzburg, UKW). Peripheral blood mononuclear cells (PBMC) were isolated by centrifugation over Ficoll-Hypaque (Sigma, St. Louis, Mo.).

Cell Lines

The 293T, MDA-MB231, T-47D, 786-O and U266 cell lines were obtained from the American Type Culture Collection. Luciferase expressing lines were derived by lentiviral transduction of the above-mentioned cell lines with the firefly (P. pyralis) luciferase (ffluc)-gene. The cells were cultured in Dulbecco's modified Eagle's medium supplemented with 10% fetal calf serum and 100 U/ml penicillin/streptomycin or RPMI-1640 medium supplemented with 10% fetal calf serum and 100 U/mL penicillin/streptomycin, respectively.

Generation, Enrichment and Expansion of CAR Knock-In T Cells.

Virus-free knock-in of genes of interest into the T cell receptor locus of primary human T cells has recently been described [62], which is hereby incorporated by reference in its entirety for all purposes. All CAR constructs encoded a truncated epidermal growth factor receptor (EGFRt; also known as tEGFR), see reference [60], which is hereby incorporated by reference in its entirety for all purposes, downstream of the CAR. The genes were linked by a T2A ribosomal skip element.

HDR templates containing two homology sequences for the human T cell receptor locus alpha chain (TRAC), CAR CDS and EGFRt were generated by custom synthesis and amplified by polymerase chain reaction (PCR). CAR knock-in T cells were generated as described [62]. In brief, CD8+ or CD4+ bulk T cells were sorted from PBMC of healthy donors and activated with anti-CD3/CD28 beads (Life Technologies). On day 3, virus-free CAR knock-in was performed by co-electroporation with hTRAC-specific sgRNA/spCas9 ribonucleoprotein (RNP) and a homology dependent repair template (HDRT) containing the XBR2-401 or X3.12 CAR coding cassettes. T cells were propagated in RPMI-1640 with 10% human serum, GlutaMAX (Life technologies), 100 U/mL penicillin-streptomycin and 50 U/mL IL-2. Trypan blue staining was performed to quantify viable T cells. After expansion, EGFRt+ T cells were enriched by magnetic activated cell sorting and expanded by polyclonal stimulation with lethally irradiated ROR2-positive U266 feeder cells.

Cytotoxicity, Cytokine Secretion, and CFSE Proliferation Assays

Target cells stably expressing firefly luciferase were incubated in triplicate at 5×10³ cells/well with effector T cells at various effector to target (E:T) ratios. The decrease in luminescence signal in wells that contained target cells and T cells was measured using a luminometer (Tecan). Specific lysis was calculated using the standard formula using TRAC KO T cells at the respective E:T for data normalization.

For the analysis of cytokine secretion, 5×10⁴ T cells were plated in triplicate wells with target cells at a ratio of 4:1 and IFN-γ, and IL-2 measured by ELISA (Biolegend) in supernatant removed after a 24-hour incubation.

For proliferation analysis, 5×10⁴ T cells were labeled with 0.2 μM carboxyfluorescein succinimidyl ester (CFSE, Invitrogen), washed and plated in triplicate wells with lethaly irradiated target cells at a ratio of 4:1 in CTL medium without exogenous cytokines. After 72 h of incubation, cells were labeled with anti-CD3 or anti-CD4 or anti-CD8 mAb and 7-AAD to exclude dead cells from analysis. Samples were analyzed by flow cytometry and cell division of live T cells assessed by CFSE dilution.

For xCELLigence killing assays, adherent tumor cells were seeded, allowed to adhere for 16 h and incubated with CAR-T cells at an E:T ratio of 1:10 in triplicates. Impedance values were measured in 15-minute intervals for >96 h.

Results

Generation, Detection and Enrichment of ROR2 CAR Knock-In T Cells

In order to investigate the impact of binding affinity (see FIG. 10) on CAR functionality under controlled conditions, we decided to employ CAR hTRAC locus knock-ins. This approach allowed us to investigate the impact of binding affinities on CAR-T cell functionality in the presence of a single CAR copy in the genome, offsetting the added benefit of multiple CAR insertions into the T cells genome.

PBMCs from healthy donors were isolated by Ficoll-Hypaque density gradient centrifugation and bulk CD4+ or CD8+ human T cells were extracted from this cell population using MACS. Directly after isolation the T cells were activated with CD3/28 Dynabeads for two days and CAR knock-in was performed as described previously. The Dynabeads were removed 4 days after knock-in and at day 10 the T cells were enriched for EGFRt-positive cells by labeling with a biotinylated monoclonal αEGFR antibody and MACS with anti-biotin microbeads. After the enrichment, the EGFRt-positive fraction ROR2-CAR knock-in T cells was reproducibly around 75 to 85%. However, the median fluorescence intensity (MFI) was significantly lower as compared to lenti-virally generated ROR2 CAR-T cells, indicating a more tightly controlled CAR expression pattern upon integration into the TRAC locus (FIG. 21, FIGS. 25A and 25B). In human T cells, CD3 and the TCR are delivered to the cell surface together. In the absence of the TCR, CD3 is also no longer present on the cell surface, making CD3 a suitable surrogate marker for TCR knock-out efficiency. (See FIGS. 25A and 25B, right side).

Cytolytic Activity of ROR2 CAR-T Cells

CAR-T cells were generated as described above and their cytolytic activity was assessed in 24 h cytotoxicity assays against the ROR2-positive and ffluc-expressing target cell lines T-47D, 786-O and U266 (FIG. 20). No specific lysis was detected against ROR2-negative MDA-MB-231 controls. X3.12-based CAR knock-in T cells consistently outperformed XBR2-401-based CAR T cells in this setting (FIG. 26A). The assay was repeated under the same conditions for n=3 independent healthy donor (FIG. 26B).

Additionally, the xCELLigence platform was utilized to investigate the effect of CAR binding affinities on CAR-T cell functionality in more detail. In order to challenge the cells, we decided to employ low E:T ratios (1:10). Surprisingly, XBR2-401-based CAR knock-in T cells were unable to control 786-O tumor growth under these conditions, whereas X3.12-based CAR knock-in T cells efficiently eradicated 786-O cells. (FIG. 26C)

Effector Cytokine Secretion Following ROR2-Specific Activation of ROR2 CAR-T Cells

CD4+ or CD8+ CAR-T cells were generated as described above and co-cultured with lethally irradiated ROR2-expressing target cell lines at an E:T ratio of 4:1 for 24 h. After the incubation the cell culture supernatant was collected and analyzed for the presence of the effector cytokines IL-2 and IFN-γ by ELISA. As controls, the cells were co-cultured with ROR2-negative MDA-MB231 cells or in absence of any target cell (media control). For controlling the general ability of the CAR-T cells to produce the effector cytokines of interest the cells were polyclonally stimulated with a combination of the protein kinase C (PKC)/NF-κB-activator phorbol 12-myristate 13-acetate (PMA) and the Ca2+ ionophore ionomycin. The assay procedure was repeated for up to n=3 unrelated healthy T cells donors and the measured cytokine concentrations were used for group analysis. (FIG. 27)

Both, IFN-γ and IL-2 were exclusively detected in samples that included ROR2-positive target cells. Again, the cytokine secretion was highly dependent on antigen expression levels (see FIG. 20), with 786-O and U266 inducing the highest cytokine secretion. Particularly for antigen low (U266) and very low (T-47D) target cells, the general correlation between high binder affinity and high cytokine secretion was true. This effect was particularly clear for T-47D cells. Cytokine levels secreted by XBR2-401 based CAR knock-in T cells were just barely above background, whereas X3.12-based CAR knock-in T cells consistently secreted 300-1200 μg/mL IL-2 and 300-2000 μg/mL IFN-γ.

Proliferation of ROR2 CAR-T Cells

CD4+ or CD8+ CAR-T cells were generated as described, labeled with CFSE and co-cultured with lethally irradiated ROR2-expressing target cell lines at an E:T ratio of 4:1 for 72 h in the absence of exogenous cytokines. After the incubation time the T cells were collected and analyzed for CFSE dilution by flow cytometry. As a negative control, the CAR-T cells were co-cultured with ROR2-negative MDA-MB-231 cells or medium and as a positive control in the presence of 50 UI/ml IL-2.

ROR2-negative MDA-MB-231 and medium alone caused no proliferation of T cells expressing any of the two CARs. However, high antigen-dependent cell proliferation was observed upon CAR-T cell activation via their respective CAR using ROR2-positive target cells (FIG. 28). These findings demonstrate that the detected proliferation of ROR2 CAR-T cells was mediated by the CAR, in response to stimulation by ROR2-positive cells. In all cases, X3.12-based CAR knock-in T cells showed higher proliferation than XBR2-401-based CAR knock-in T cells against ROR2-positive tumor cells.

Conclusion

Taken together, our findings underline the superiority of X3.12-based CAR knock-in T cells over XBR2-401 based ones. X3.12-based CAR knock-in T cells consistently outperformed XBR2-401-based CAR knock-in T cells in all assays.

Our data indicates that the increased affinity of X3.12 provides CAR T cells with an additional advantage in antigen-low situations, which have been reported to be a leading cause for tumor relapse after CAR-T cell treatment.

### Example 4: Functional Validation of Humanized ROR2 CARs

Materials and Methods:

Human Subjects

Blood samples were obtained from healthy donors who provided written informed consent to participate in research protocols approved by the Institutional Review Board of the University of Würzburg (Universitätsklinikum Würzburg, UKW). Peripheral blood mononuclear cells (PBMC) were isolated by centrifugation over Ficoll-Hypaque (Sigma, St. Louis, Mo.).

Cell Lines

The HEK-293T, T-47D, 786-O and U266 cell lines were obtained from the American Type Culture Collection. OPM-2 cells obtained from the DSMZ. Luciferase expressing lines were derived by lentiviral transduction of the above-mentioned cell lines with the firefly (P. pyralis) luciferase (ffluc)-gene. The cells were cultured in Dulbecco's modified Eagle's medium supplemented with 10% fetal calf serum and 100 U/ml penicillin/streptomycin or RPMI-1640 medium supplemented with 10% fetal calf serum and 100 U/mL penicillin/streptomycin, respectively.

Lentiviral Vector Construction, Preparation of Lentivirus, and Generation of CAR-T Cells

The construction of epH IV7 lentiviral vectors containing ROR2-specific CARs with optimal spacers and a cd28 or 4-1BB costimulatory domain has been described, see reference [58, 59], which is hereby incorporated by reference in its entirety for all purposes. All CAR constructs encoded a truncated epidermal growth factor receptor (EGFRt; also known as tEGFR), see reference [60], which is hereby incorporated by reference in its entirety for all purposes, downstream of the CAR. The genes were linked by a T2A ribosomal skip element.

CAR/EGFRt and ffluc/eGFP-encoding lentivirus supernatants were produced in 293T cells co-transfected with each of the lentiviral vector plasmids and the packaging vectors pCHGP-2, pCMV-Rev2 and pCMV-G using Calphos transfection reagent (Clontech, Mountain View, Calif.). Medium was changed 16 h after transfection, and lentivirus collected after 72 h. CAR-T cells were generated as described [61]. In brief, CD8+ or CD4+ bulk T cells were sorted from PBMC of healthy donors, activated with anti-CD3/CD28 beads (Life Technologies), and transduced with lentiviral supernatant. Lentiviral transduction was performed on day 2 by spinoculation, and T cells propagated in RPMI-1640 with 10% human serum, GlutaMAX (Life technologies), 100 U/mL penicillin-streptomycin and 50 U/mL IL-2. Trypan blue staining was performed to quantify viable T cells. After expansion, EGFRt+ T cells were enriched by magnetic activated cell sorting and expanded by polyclonal stimulation with the CD3-specific Okt3 antibody and irradiated allogeneic PBMC and EBV-LCL feeder cells.

Cytotoxicity, Cytokine Secretion, and CFSE Proliferation Assays

Target cells stably expressing firefly luciferase were incubated in triplicate at 5×10³ cells/well with effector T cells at various effector to target (E:T) ratios. The decrease in luminescence signal in wells that contained target cells and T cells was measured using a luminometer (Tecan). Specific lysis was calculated using the standard formula using untransduced T cells at the respective E:T to offset TCR mediated cytolysis. For sequential killing assays, T cells were re-challenged with fresh tumor cells every 24 h at defined E:T ratios.

For the analysis of cytokine secretion, 5×10⁴ T cells were plated in triplicate wells with target cells at a ratio of 4:1 and IFN-γ measured by ELISA (Biolegend) in supernatant removed after a 24-hour incubation.

For proliferation analysis, 5×10⁴ T cells were labeled with 0.2 μM carboxyfluorescein succinimidyl ester (CFSE, Invitrogen), washed and plated in triplicate wells with lethally irradiated target cells at a ratio of 4:1 in CTL medium without exogenous cytokines. After 72 h of incubation, cells were labeled with anti-CD3 or anti-CD4 or anti-CD8 mAb and 7-AAD to exclude dead cells from analysis. Samples were analyzed by flow cytometry and cell division of live T cells assessed by CFSE dilution. Proliferation index, expansion index and the fraction of cells undergoing any respective number of cell divisions were extracted using FlowJo (Tree Star).

Results

Generation, Detection and Enrichment of ROR2 CAR-T Cells

PBMCs from healthy donors were isolated by Ficoll-Hypaque density gradient centrifugation and bulk CD4+ or CD8+ human T cells were extracted from this cell population using MACS. Directly after isolation the T cells were activated with CD3/28 Dynabeads for one day and then transduced by spinoculation with lentiviral vectors encoding the X3.12, hX3.12.5, or hX3.12.6-based ROR2-specific CARs at a multiplicity of infection (MOI) of 3. The Dynabeads were removed 4 days after transduction and at day 10 the T cells were enriched for EGFRt-positive cells by labeling with a biotinylated monoclonal αEGFR antibody and MACS using anti-biotin microbeads. After the enrichment, the EGFRt-positive fraction reproducibly accounted for over 89% of total cells (FIGS. 29A and 29B).

Cytolytic Activity of Humanized ROR2 CAR-T Cells

CAR-T cells were generated as described above and their cytolytic activity was assessed in 24 h cytotoxicity assays against the ROR2-positive and ffluc-expressing target cell lines T-47D, 786-O and U266 (see FIG. 30 A), as well as ROR2-negative OPM-2 cells. The assay was repeated under the same conditions for n=3 independent healthy donor (FIG. 30 B). In line with previous studies utilizing hX3.12.5 and hX3.12.6 in biAb format, hX3.12.5 showed reduced cytolytic activity as compared to both X3.12 and hX3.12.6 derived CAR-T cells. This effect was more pronounced in antigen-low settings (T-47D) and decreased in the presence of medium (786-O) and high (U-266) antigen densities. No specific lysis was detected against ROR2-negative OPM-2 controls (FIG. 31). Statistics are based on repeated-measures ANOVA and pairwise comparison by Dunnet's multiple comparisons test (n.s. not significant, *=p<0.05, **=p<0.01, ***, p<0.001).

Effector Cytokine Secretion Following ROR2-Specific Activation of ROR2 CAR-T Cells

CD4+ or CD8+ X3.12, hX3.12.5 and hX3.12.6-based ROR2-specific CAR-T cells were generated as described above and co-cultured with ROR2-expressing target cell lines at an E:T ratio of 4:1 for 24 h. After the incubation the cell culture supernatant was collected and analyzed for the presence of the effector cytokine IFN-γ by ELISA. As controls, the cells were co-cultured with ROR2-negative OPM-2 cells or in absence of any target cell (media control). For controlling the general ability of the CAR-T cells to produce the effector cytokines of interest the cells were polyclonally stimulated with a combination of the protein kinase C (PKC)/NF-κB-activator phorbol 12-myristate 13-acetate (PMA) and the Ca2+ ionopohore ionomycin. The assay procedure was repeated for n=3 unrelated healthy donors and the measured cytokine concentrations were used for group analysis (FIG. 32).

All three ROR2-specific CAR T cell variants showed antigen-dependent cytokine secretion profiles. Cytokine secretion levels were affected by antigen expression levels to a lesser extent than cytolysis. In general, CD4+ T cells secreted higher amounts of IFN-γ than CD8+ T cells. IFN-γ was detected exclusively in samples that included ROR2-positive targets or PMA/Iono and the average concentrations were in the range of 600-2200 μg/mL (CD4+ T cells) and 100 to 1200 μg/mL (CD8+ T cells). hX3.12.5-based ROR2-specific CAR T cells lead to lower amounts of IFN-γ secretion, in both CD4+ and CD8+ T cells. No statistically significant difference in IFN-γ secretion could be found for X3.12 and hX3.12.6-based CAR T cells. Statistics are based on repeated-measures ANOVA and pairwise comparison by Dunnet's multiple comparisons test (n.s. not significant, *=p<0.05, **=p<0.01, ***, p<0.001).

Proliferation of ROR2 CAR-T cells

CD4+ or CD8+ X3.12.-based ROR2-specific CAR-T cells were generated as described, labeled with CFSE and co-cultured with lethally irradiated ROR2-positive target cell lines at an E:T ratio of 4:1 for 72 h in the absence of exogenous cytokines. After the incubation time the T cells were collected and analyzed for CFSE dilution by flow cytometry. As a negative control, the CAR-T cells were co-cultured with ROR2-negative OPM-2 cells or medium and as a positive control in the presence of 50 UI/ml IL-2.

ROR2-negative OPM-2 and medium alone caused no T cell proliferation. However, high antigen-dependent CAR-T cell proliferation was observed for all three ROR2-specific CAR T cells against ROR2-positive target cells (FIG. 33). In line with the data obtained from cytolysis assays, proliferation levels were dependent on antigen expression levels (T-47D<786-O<U-266). Distinct differences in CAR T cell proliferation were detected at all expression levels with X3.12 and hX3.12.6-based CART cells outperforming hX3.12.5-derived CAR T cells. In line with previous findings, hX3.12.5-based CAR T cells proliferated less extensively than X3.12- and hX3.12.6-based CART cells, whereas no statistically significant difference in antigen-dependent T cell proliferation was found between X3.12 and hX3.12.6-based CARs. Statistics are based on repeated-measures ANOVA and pairwise comparison by Dunnet's multiple comparisons test (n.s. not significant, *=p<0.05, **=p<0.01, ***, p<0.001). These findings demonstrate that the detected proliferation of ROR2 CAR-T cells was mediated by the CAR, in response to stimulation by ROR2-positive cells.

Conclusion

Taken together, our findings show that humanization of the binder affects the potency of the generated CAR T cells and that a potency equivalent to that of the original binder can advantageously be achieved by optimal binder humanization. In this analysis we were able to show that humanized hX3.12.6-derived CAR-T cells are at least equally potent as CAR-T cells based on the parental clone (X3.12) with regards to antigen-dependent cytolysis, cytokine secretion and proliferation, whereas hX3.12.5-based ROR2 CAR-T cells showed reduced cytolysis, cytokine secretion and proliferation. Additionally, our data indicates that the superiority of hX3.12.6 and X3.12 over hX3.12.5 may be particularly beneficial in antigen-low settings, which have been reported to be a major reason for tumor relapse after CAR-T cell treatment.

### INDUSTRIAL APPLICABILITY

The antibodies and derivatives according to the invention can be industrially manufactured and sold as products, e.g. for the uses defined herein (e.g. for treating a cancer as defined herein), in accordance with known standards for the manufacture of pharmaceutical and diagnostic products. Accordingly, the present invention is industrially applicable.

### REFERENCES

1. Kalos, M., et al., T cells with chimeric antigen receptors have potent antitumor effects and can establish memory in patients with advanced leukemia. Sci Transl Med, 2011. 3(95): p. 95ra73.
2. Kochenderfer, J. N., et al., B-cell depletion and remissions of malignancy along with cytokine-associated toxicity in a clinical trial of anti-CD19 chimeric-antigen-receptor-transduced T cells. Blood, 2012. 119(12): p. 2709-20.
3. Kochenderfer, J. N., et al., Eradication of B-lineage cells and regression of lymphoma in a patient treated with autologous T cells genetically engineered to recognize CD19. Blood, 2010. 116(20): p. 4099-102.
4. Morgan, R. A., et al., Cancer regression in patients after transfer of genetically engineered lymphocytes. Science, 2006. 314(5796): p. 126-9.
5. Porter, D. L., et al., Chimeric antigen receptor-modified T cells in chronic lymphoid leukemia. N Engl J Med, 2011. 365(8): p. 725-33.
6. Eshhar, Z., et al., Specific activation and targeting of cytotoxic lymphocytes through chimeric single chains consisting of antibody-binding domains and the gamma or zeta subunits of the immunoglobulin and T-cell receptors. Proc Natl Acad Sci USA, 1993. 90(2): p. 720-4.
7. Kershaw, M. H., et al., Supernatural T cells: genetic modification of T cells for cancer therapy. Nat Rev Immunol, 2005. 5(12): p. 928-40.
8. Sadelain, M., I. Riviere, and R. Brentjens, Targeting tumours with genetically enhanced T lymphocytes. Nat Rev Cancer, 2003. 3(1): p. 35-45.
9. Brentjens, R. J., et al., Safety and persistence of adoptively transferred autologous CD19-targeted T cells in patients with relapsed or chemotherapy refractory B-cell leukemias. Blood, 2011. 118(18): p. 4817-28.
10. Almagro, J. C., et al., Progress and Challenges in the Design and Clinical Development of Antibodies for Cancer Therapy. Front Immunol, 2017. 8: p. 1751.
11. Masiakowski, P. and R. D. Carroll, A novel family of cell surface receptors with tyrosine kinase-like domain. J Biol Chem, 1992. 267(36): p. 26181-90.
12. Stricker, S., V. Rauschenberger, and A. Schambony, ROR-Family Receptor Tyrosine Kinases. Curr Top Dev Biol, 2017. 123: p. 105-142.
13. Matsuda, T., et al., Expression of the receptor tyrosine kinase genes, Ror1 and Ror2, during mouse development. Mech Dev, 2001. 105(1-2): p. 153-6.
14. Rebagay, G., et al., ROR1 and ROR2 in human malignancies: potentials for targeted therapy. Front Oncol, 2012. 2: p. 34.
15. Debebe, Z. and W. K. Rathmell, Ror2 as a therapeutic target in cancer. Pharmacol Ther, 2015. 150: p. 143-8.
16. Niehrs, C., The complex world of WNT receptor signalling. Nat Rev Mol Cell Biol, 2012. 13(12): p. 767-79.
17. DeChiara, T. M., et al., Ror2, encoding a receptor-like tyrosine kinase, is required for cartilage and growth plate development. Nat Genet, 2000. 24(3): p. 271-4.
18. Green, J. L., S. G. Kuntz, and P. W. Stemberg, Ror receptor tyrosine kinases: orphans no more. Trends Cell Biol, 2008. 18(11): p. 536-44.
19. Al-Shawi, R., et al., Expression of the Ror1 and Ror2 receptor tyrosine kinase genes during mouse development. Dev Genes Evol, 2001. 211(4): p. 161-71.
20. Forrester, W. C., The Ror receptor tyrosine kinase family. Cell Mol Life Sci, 2002. 59(1): p. 83-96.
21. Morioka, K., et al., Orphan receptor tyrosine kinase ROR2 as a potential therapeutic target for osteosarcoma. Cancer Sci, 2009. 100(7): p. 1227-33.
22. Wright, T. M., et al., Ror2, a developmentally regulated kinase, promotes tumor growth potential in renal cell carcinoma. Oncogene, 2009. 28(27): p. 2513-23.
23. Wright, T. M. and W. K. Rathmell, Identification of Ror2 as a hypoxia-inducible factor target in von Hippel-Lindau-associated renal cell carcinoma. J Biol Chem, 2010. 285(17): p. 12916-24.
24. Henry, C., et al., Expression of the novel Wnt receptor ROR2 is increased in breast cancer and may regulate both beta-catenin dependent and independent Wnt signalling. J Cancer Res Clin Oncol, 2015. 141(2): p. 243-54.
25. Frenquelli, M., et al., The WNT receptor ROR2 drives the interaction of multiple myeloma cells with the microenvironment through AKT activation. Leukemia, 2020. 34(1): p. 257-270.
26. Edris, B., et al., ROR2 is a novel prognostic biomarker and a potential therapeutic target in leiomyosarcoma and gastrointestinal stromal tumour. J Pathol, 2012. 227(2): p. 223-33.
27. Bayerlova, M., et al., Ror2 Signaling and Its Relevance in Breast Cancer Progression. Front Oncol, 2017. 7: p. 135.
28. O'Connell, M. P., et al., Hypoxia induces phenotypic plasticity and therapy resistance in melanoma via the tyrosine kinase receptors ROR1 and ROR2. Cancer Discov, 2013. 3(12): p. 1378-93.
29. Peng, H., et al., Mining naive rabbit antibody repertoires by phage display for monoclonal antibodies of therapeutic utility. J Mol Biol, 2017. 429(19): p. 2954-2973.
30. Rader, C., Peng, H., and Li, X., PCT/US2017/014370. 2017.
31. Baca, M., et al., Antibody humanization using monovalent phage display. J Biol Chem, 1997. 272(16): p. 10678-84.
32. Altschul, S. F., et al., Basic local alignment search tool. J Mol Biol, 1990. 215(3): p. 403-10.
33. Altschul, S. F., et al., Gapped BLAST and PSI-BLAST: a new generation of protein database search programs. Nucleic Acids Res, 1997. 25(17): p. 3389-402.
34. Qi, J., et al., Potent and selective antitumor activity of a T cell-engaging bispecific antibody targeting a membrane-proximal epitope of ROR1. PNAS, 2018. 115(24): p. E5467-E5476.
35. Park, H., F. G. Adsit, and J. C. Boyington, The 1.4 angstrom crystal structure of the human oxidized low density lipoprotein receptor lox-1. J Biol Chem, 2005. 280(14): p. 13593-9.
36. Otwinowski, Z. and W. Minor, Macromolecular Crystallography, Part A. Methods in Enzymology. Vol. 276. 1997: Academic Press.
37. Vagin A and Teplyakov A, MOLREP: an automated program for molecular replacement. Journal of Applied Crystallography, 1997. 30(6): p. 1022-1025.
38. Adams P D, et al., PHENIX: building new software for automated crystallographic structure determination. Acta Crystallogr D Biol Crystallogr., 2002. 58(Pt 11): p. 1948-54. Epub 2002 Oct. 21.
39. Emsley, P. and K. Cowtan, Coot: model-building tools for molecular graphics. Acta Crystallogr D Biol Crystallogr, 2004. 60(Pt 12 Pt 1): p. 2126-32.
40. Pettersen, E. F., et al., UCSF Chimera—a visualization system for exploratory research and analysis. J Comput Chem, 2004. 25(13): p. 1605-12.
41. Brunger, A. T., et al., Crystallography & NMR system: A new software suite for macromolecular structure determination. Acta Crystallogr D Biol Crystallogr, 1998. 54(Pt 5): p. 905-21.
42. Brunger, A. T., Version 1.2 of the Crystallography and NMR system. Nat Protoc, 2007. 2(11): p. 2728-33.
43. Chen, V. B., et al., MolProbity: all-atom structure validation for macromolecular crystallography. Acta Crystallogr D Biol Crystallogr, 2010. 66(Pt 1): p. 12-21.
44. Rader, C., Generation and selection of rabbit antibody libraries by phage display. Methods Mol Biol, 2009. 525: p. 101-28, xiv.
45. Zhang, Y. F. and M. Ho, Humanization of rabbit monoclonal antibodies via grafting combined Kabat/IMGT/Paratome complementarity-determining regions: Rationale and examples. mAbs, 2017. 9(3): p. 419-429.
46. Stahl, S. J., et al., Generation and characterization of a chimeric rabbit/human Fab for co-crystallization of HIV-1 Rev. J Mol Biol, 2010. 397(3): p. 697-708.
47. Freeth, J. and J. Soden, New Advances in Cell Microarray Technology to Expand Applications in Target Deconvolution and Off-Target Screening. SLAS Discov, 2019: p. 10.1177/2472555219897567.
48. Robinson, H. R., et al., A CD19/CD3 bispecific antibody for effective immunotherapy of chronic lymphocytic leukemia in the ibrutinib era. Blood, 2018. 132(5): p. 521-532.
49. Merchant, A. M., et al., An efficient route to human bispecific IgG. Nat Biotechnol, 1998. 16(7): p. 677-81.
50. Lee, C. H., et al., Engineering of a human kringle domain into agonistic and antagonistic binding proteins functioning in vitro and in vivo. PNAS, 2010. 107(21): p. 9567-71.
51. Wang, M., et al., Solution structure of the complex of VEK-30 and plasminogen kringle 2. J Struct Biol, 2010. 169(3): p. 349-59.
52. Jones, T. D., et al., The INNs and outs of antibody nonproprietary names. mAbs, 2016. 8(1): p. 1-9.
53. Menzen, T. and W. Friess, Temperature-ramped studies on the aggregation, unfolding, and interaction of a therapeutic monoclonal antibody. J Pharm Sci, 2014. 103(2): p. 445-55.
54. McConnell, A. D., et al., A general approach to antibody thermostabilization. mAbs, 2014. 6(5): p. 1274-82.
55. Hristodorov, D., et al., Generation and comparative characterization of glycosylated and aglycosylated human IgG1 antibodies. Mol Biotechnol, 2013. 53(3): p. 326-35.
56. Atwell, S., et al., Stable heterodimers from remodeling the domain interface of a homodimer using a phage display library. J Mol Biol, 1997. 270(1): p. 26-35.
57. Zhu, Z., G. D. Lewis, and P. Carter, Engineering high affinity humanized anti-p185HER2/anti-CD3 bispecific F(ab)2 for efficient lysis of p185HER2 overexpressing tumor cells. IJC, 1995. 62(3): p. 319-324.
58. Maus, M. V., et al., T cells expressing chimeric antigen receptors can cause anaphylaxis in humans. Cancer Immunol Res, 2013. 1(1): p. 26-31.
59. Sommermeyer, D., et al., Fully human CD19-specific chimeric antigen receptors for T-cell therapy. Leukemia, 2017. 31(10): p. 2191-2199.
60. Wang, X., et al., A transgene-encoded cell surface polypeptide for selection, in vivo tracking, and ablation of engineered cells. Blood, 2011. 118(5): p. 1255-63.
61. Hudecek, M., et al., Receptor affinity and extracellular domain modifications affect tumor recognition by ROR1-specific chimeric antigen receptor T cells. Clin Cancer Res, 2013. 19(12): p. 3153-64.
62. Roth, T. L., et al., Reprogramming human T cell function and specificity with non-viral genome targeting. Nature, 2018. 559(7714): p. 405-409.
63. WO 2017/127702 A1
64. D. T. Rodgers, M. Mazagova, E. N. Hampton, Y. Cao, N. S. Ramadoss, I. R. Hardy, A. Schulman, J. Du, F. Wang, O. Singer, J. Ma, V. Nunez, J. Shen, A. K. Woods, T. M. Wright, P. G. Schultz, C. H. Kim, T. S. Young, Proc Natl Acad Sci USA 2016, 113, E459-468.
65. J. S. Ma, J. Y. Kim, S. A. Kazane, S. H. Choi, H. Y. Yun, M. S. Kim, D. T. Rodgers, H. M. Pugh, O. Singer, S. B. Sun, B. R. Fonslow, J. N. Kochenderfer, T. M. Wright, P. G. Schultz, T. S. Young, C. H. Kim, Y. Cao, Proc Natl Acad Sci USA 2016, 113, E450-458.
66. Y. Cao, D. T. Rodgers, J. Du, I. Ahmad, E. N. Hampton, J. S. Ma, M. Mazagova, S. H. Choi, H. Y. Yun, H. Xiao, P. Yang, X. Luo, R. K. Lim, H. M. Pugh, F. Wang, S. A. Kazane, T. M. Wright, C. H. Kim, P. G. Schultz, T. S. Young, Angew Chem Int Ed Engl 2016, 55, 7520-7524.
67. L. J. B. Brandt, M. B. Barnkob, Y. S. Michaels, J. Heiselberg, T. Barington, Front Immunol 2020, 26 Feb. 2020 (doi.org/10.3389/fimmu.2020.00326).
68. Kabat E. A., Wu, T. T., Perry, H., Gottesman, K. and Foeller, C. (1991) Sequences of Proteins of Immunological Interest, Fifth Edition. NIH Publication No. 91-3242.

1. An antibody capable of binding to human ROR2 or a derivative thereof capable of binding to human ROR2, wherein the antibody or derivative thereof comprises a light chain variable domain and a heavy chain variable domain, wherein the heavy chain variable domain comprises a CDR3 sequence having an amino acid sequence selected from the group consisting of SEQ ID NO: 23, SEQ ID NO: 24 and SEQ ID NO: 25, and wherein said CDR3 sequence does not have the amino acid sequence of SEQ ID NO: 36.

2. The antibody or derivative thereof of claim 1, wherein the heavy chain variable domain comprises a CDR3 sequence having an amino acid sequence selected from the group consisting of SEQ ID NO: 24 and SEQ ID NO: 25, wherein, optionally, the heavy chain variable domain comprises a CDR3 sequence having the amino acid sequence of SEQ ID NO: 24.

3. (canceled)

4. The antibody or derivative thereof of claim 1, wherein the heavy chain variable domain comprises a CDR3 sequence having an amino acid sequence selected from the group consisting of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 37, and SEQ ID NO: 38.

5. The antibody or derivative thereof of claim 1, wherein the heavy chain variable domain comprises a CDR3 sequence having an amino acid sequence selected from the group consisting of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35, wherein, optionally, the heavy chain variable domain comprises a CDR3 sequence having the amino acid sequence of SEQ ID NO: 26.

6. (canceled)

7. The antibody or derivative thereof of claim 1, wherein the light chain variable domain comprises a CDR3 sequence having the amino acid sequence of SEQ ID NO: 43 or a CDR3 sequence which differs from the amino acid sequence of SEQ ID NO: 43 by not more than two amino acid residues, wherein, optionally, the light chain variable domain comprises a CDR3 sequence having the amino acid sequence of SEQ ID NO: 43 or a CDR3 sequence which differs from the amino acid sequence of SEQ ID NO: 43 by not more than one amino acid residue, wherein, optionally, the light chain variable domain comprises a CDR3 sequence having the amino acid sequence of SEQ ID NO: 43.

8-9. (canceled)

10. The antibody or derivative thereof of claim 1, wherein the heavy chain variable domain comprises a CDR2 sequence having the amino acid sequence of SEQ ID NO: 45 or a CDR2 sequence which differs from the amino acid sequence of SEQ ID NO: 45 by not more than two amino acid residues, wherein, optionally, the heavy chain variable domain comprises a CDR2 sequence having the amino acid sequence of SEQ ID NO: 45 or a CDR2 sequence which differs from the amino acid sequence of SEQ ID NO: 45 by not more than one amino acid residue, wherein, optionally, the heavy chain variable domain comprises a CDR2 sequence having the amino acid sequence of SEQ ID NO: 45.

11-12. (canceled)

13. The antibody or derivative thereof of claim 1, wherein the light chain variable domain comprises a CDR1 sequence having the amino acid sequence of SEQ ID NO: 41 or a CDR1 sequence which differs from the amino acid sequence of SEQ ID NO: 41 by not more than two amino acid residues, wherein, optionally, the light chain variable domain comprises a CDR1 sequence having the amino acid sequence of SEQ ID NO: 41 or a CDR1 sequence which differs from the amino acid sequence of SEQ ID NO: 41 by not more than one amino acid residue, wherein, optionally, the light chain variable domain comprises a CDR1 sequence having the amino acid sequence of SEQ ID NO: 41.

14-15. (canceled)

16. The antibody or derivative thereof of claim 1, wherein the light chain variable domain comprises a CDR2 sequence having the amino acid sequence of SEQ ID NO: 42 or a CDR2 sequence which differs from the amino acid sequence of SEQ ID NO: 42 by not more than two amino acid residues, wherein, optionally, the light chain variable domain comprises a CDR2 sequence having the amino acid sequence of SEQ ID NO: 42 or a CDR2 sequence which differs from the amino acid sequence of SEQ ID NO: 42 by not more than one amino acid residue, wherein, optionally, the light chain variable domain comprises a CDR2 sequence having the amino acid sequence of SEQ ID NO: 42.

17-18. (canceled)

19. The antibody or derivative thereof of claim 1, wherein the heavy chain variable domain comprises a CDR1 sequence having the amino acid sequence of SEQ ID NO: 44 or a CDR1 sequence which differs from the amino acid sequence of SEQ ID NO: 44 by not more than two amino acid residues, wherein, optionally, the heavy chain variable domain comprises a CDR1 sequence having the amino acid sequence of SEQ ID NO: 44 or a CDR1 sequence which differs from the amino acid sequence of SEQ ID NO: 44 by not more than one amino acid residue, wherein, optionally, the heavy chain variable domain comprises a CDR1 sequence having the amino acid sequence of SEQ ID NO: 44.

20-21. (canceled)

22. The antibody or derivative of claim 1, wherein said antibody or derivative is capable of binding to human ROR2 with higher affinity than a corresponding antibody or derivative comprising a light chain variable domain having the amino acid sequence of SEQ ID NO: 2 and a heavy chain variable domain having the amino acid sequence of SEQ ID NO: 1, as determined by surface plasmon resonance measurements.

23. The antibody or derivative thereof of claim 1, wherein (i) the heavy chain variable domain comprises the amino acid sequence of SEQ ID NO:
3 and the light chain variable domain comprises the amino acid sequence of SEQ ID NO: 4, or (ii) the heavy chain variable domain comprises the amino acid sequence of SEQ ID NO: 5 and the light chain variable domain comprises the amino acid sequence of SEQ ID NO: 6, or (iii) the heavy chain variable domain comprises the amino acid sequence of SEQ ID NO: 7 and the light chain variable domain comprises the amino acid sequence of SEQ ID NO: 6, which is optionally a humanized antibody or derivative thereof.

24. (canceled)

25. The antibody or derivative thereof of claim 1, wherein (ii) the heavy chain variable domain comprises the amino acid sequence of SEQ ID NO: 5 and the light chain variable domain comprises the amino acid sequence of SEQ ID NO: 6, or (iii) the heavy chain variable domain comprises the amino acid sequence of SEQ ID NO: 7 and the light chain variable domain comprises the amino acid sequence of SEQ ID NO: 6.

26. The antibody or derivative thereof of claim 1, which is a bispecific antibody or derivative thereof, wherein the bispecific antibody or derivative thereof is also capable of binding to human CD3.

27. (canceled)

28. The antibody or derivative thereof of claim 1, which is a derivative of the antibody, wherein the derivative is optionally
(a) an antibody fragment,
(b) a CAR, or
(c) a derivative comprising an adaptor for a universal CAR.

29-32. (canceled)

33. A nucleic acid encoding the antibody or derivative of claim 1.

34. The nucleic acid of claim 33, wherein the nucleic acid is an mRNA, or a DNA, wherein the DNA is optionally a minicircle or plasmid DNA.

35-36. (canceled)

37. A recombinant immune cell comprising the CAR of claim 28 (b) and/or comprising a nucleic acid according to any one of claims 33-36 which encodes the CAR of claim 28 (b).

38. The recombinant immune cell of claim 37, wherein said immune cell is a CD8+ killer T cell, a CD4+ helper T cell, a naive T cell, a memory T cell, a central memory T cell, an effector memory T cell, a memory stem T cell, an invariant T cell, an NKT cell, a cytokine induced killer T cell, a g/d T cell, a natural killer cell, a monocyte, a macrophage, a dendritic cell, or a granulocyte.

39. The recombinant immune cell of claim 37, wherein the immune cell is a T-cell.

40. A pharmaceutical composition comprising:
(i) the antibody or derivative thereof of any one of claims 28(a) or claim 28(c);
(ii) a nucleic acid according to any one of claims 33-36 which encodes the CAR of claim 28(b);
(iii) the recombinant immune cell of any one of claims 37-39; or
(iv) a combination of (i) and (ii), a combination of (i) and (iii), or a combination of (i) to (iii).

41. A pharmaceutical composition according to claim 40, for use in the treatment of cancer.

42. The pharmaceutical composition for use of claim 41, wherein the cancer is a ROR2-expressing cancer, wherein the cancer is optionally a hematologic cancer or a solid cancer.

43-44. (canceled)

45. The pharmaceutical composition for use of claim 41, wherein the cancer is selected from the group consisting of multiple myeloma, renal cell carcinoma, pancreatic cancer, sarcoma, glioblastoma and mammary carcinoma.

46. Use of a compound for the diagnosis of cancer, wherein the compound is (i) the antibody or derivative thereof of claim 26;
(ii) a nucleic acid according to any one of claims 33-36 which encodes the CAR of claim 28(b);
(iii) the recombinant immune cell of any one of claims 37-39; or
(iv) a combination of (i) and (ii), a combination of (i) and (iii), or a combination of (i) to (iii).

47. Use of a compound for determining the susceptibility of a cancer to therapy, wherein the compound is
(i) the antibody or derivative thereof of claim 1;
(ii) a nucleic acid according to any one of claims 33-36 which encodes the CAR of claim 28(b);
(iii) the recombinant immune cell of any one of claims 37-39; or
(iv) a combination of (i) and (ii), a combination of (i) and (iii), or a combination of (i) to (iii).
